(19) United States
(12) Patent Application Publication
(10) Pub. No.: US 2014/0248687 A1
(43) Pub. Date: Sep. 4, 2014

(54) METHODS FOR EXPRESSING POLYPEPTIDES IN HYPERTHERMOPHILES

(71) Applicants: University of Georgia Research Foundation, Inc., Athens, GA (US); North Carolina State University, Raleigh, NC (US)

(72) Inventors: Robert M. Kelly, Raleigh, NC (US); Michael W.W. Adams, Athens, GA (US); Mirko Basen, Athens, GA (US); Gina Pires Lipscomb, Athens, GA (US); Angeli Menon, Athens, GA (US)

(73) Assignees: University of Georgia Research Foundation, Inc., Athens, GA (US); North Carolina State University, Raleigh, NC (US)

(21) Appl. No.: 14/355,362

(22) PCT Filed: Nov. 2, 2012
(86) PCT No.: PCT/US2012/063289

§ 371 Date: Apr. 30, 2014

Related U.S. Application Data
(60) Provisional application No. 61/555,683, filed on Nov. 4, 2011.

(51) Int. Cl. C12N 15/74

(52) U.S. Cl. CPC C12N 15/74
USPC 435/252.3

(57) ABSTRACT

Provided herein are genetically engineered archaea. A genetically engineered archaea includes a heterologous polynucleotide that has a promoter operably linked to a coding region, where the coding region encodes a polypeptide having optimal activity below the optimum growth temperature (Tₒₚₜ) of the genetically engineered archaeon. Also provided herein are methods for using genetically engineered archaea and cell-free extracts of such genetically engineered archaea. In one embodiment, the methods include culturing a genetically engineered archaeon at a temperature that is at least 20° C. below the Tₒₚₜ of the genetically engineered archaeon, such that the activity in the genetically engineered archaeon of a polypeptide encoded by the coding region is increased compared to the activity in the genetically engineered archaeon of the polypeptide during growth at a second temperature that is at or near the Tₒₚₜ of the genetically engineered archaeon.

### CROSS-REFERENCE TO RELATED APPLICATIONS

This application claims the benefit of U.S. Provisional Application Ser. No. 61/555,683, filed Nov. 4, 2011, which is incorporated by reference herein.

### GOVERNMENT FUNDING

The present invention was made with government support under Grant No. DE-PS02-06ER64304 and Grant No. DE-AR0000081, each awarded by the Department of Energy, and under Grant No. BES-0617272, awarded by the National Science Foundation. The Government has certain rights in this invention.

### BACKGROUND

Since the discovery of hyperthermophiles in the 1980s (Stetter, 2006, Extremophiles 10:357-362), hyperthermophiles have attracted a great deal of attention due to their ability to grow optimally at temperatures above 80° C. Virtually all are classified within the archaeal domain rather than the bacterial domain. In addition to their evolutionary implications, hyperthermostable enzymes are of high biotechnological interest (Barnard et al., 2010, Environ. Technol. 31:871-888, Blumer-Schuette et al., 2008. Curr. Opin. Biotechnol. 19:210-217, Atomi et al., 2011. Curr. Opin. Biotechnol. 22:618-626), since many industrial processes are facilitated by elevated temperatures and organisms that grow under such conditions can be used without risk of contamination (Blumer-Schuette et al., 2008. Curr. Opin. Biotechnol. 19:210-217). Although the ability to metabolically engineer microorganisms is a prerequisite for their utility as whole-cell biocatalysts, the genetic manipulation of hyperthermophiles is a very recent development. Targeted modifications of the chromosome have been reported for those microorganisms growing optimally near 80° C. or so, which include Sulfolobus acidocaldarius (optimal growth temperature [Tₒₚₜ] of 80° C.) and the related species Sulfolobus solfataricus (Tₒₚₜ of 75° C.) (Schelert et al. 2004. J. Bacteriol. 186:427-437, Wagner et al. 2009. Biochem. Soc. Trans. 37:97-101), Thermococcus kodakarensis (Tₒₚₜ of 85° C.) (Sato et al., 2003. J. Bacteriol. 185:210-220), and Pyrococcus furiosus, for an organism that grows optimally near 100° C. (Lipscomb et al. 2011. Environ. Microbiol. 77:2232-2238). P. furiosus is one of the best-studied hyperthermophiles, belonging to the same family as T. kodakarensis but with a much higher optimal growth temperature. P. furiosus is a strict anaerobe and obtains carbon and energy for growth by the fermentation of carbohydrates and peptides with organic acids, CO₂, and H₂ as end products (Chou et al., 2008. Metab. Eng. 10:394-104).

### SUMMARY OF THE INVENTION

Provided herein are genetically engineered archaea and methods for using genetically engineered archaea. In one embodiment, a method includes culturing a genetically engineered archaeon, wherein the genetically engineered archaeon includes a heterologous polynucleotide that has a promoter operably linked to a coding region. The culturing is at a temperature that is at least 20° C. below the optimum growth temperature (Tₒₚₜ) of the genetically engineered archaeon. The method further includes maintaining the genetically engineered archaeon at the temperature, wherein activity in the genetically engineered archaeon of a polypeptide encoded by the coding region is increased compared to the activity in the genetically engineered archaeon of the polypeptide during growth at a second temperature that is at or near the Tₒₚₜ. of the genetically engineered archaeon.

In one embodiment, the method includes culturing a genetically engineered archaeon, wherein the genetically engineered archaeon includes a heterologous polynucleotide having a promoter operably linked to a coding region. The culturing of the genetically engineered archaeon is at a first temperature that is within 10° C. of the optimum growth temperature (Tₒₚₜ) of the genetically engineered archaeon. The method further includes shifting the culture to a second temperature that is at least 20° C. below the Tₒₚₜ of the genetically engineered archaeon, and maintaining the genetically engineered archaeon at the second temperature, wherein activity in the genetically engineered archaeon of a polypeptide encoded by the coding region is increased compared to the activity in the genetically engineered archaeon of the polypeptide during growth at the first temperature.

The genetically engineered archaeon may be, for instance, Thermococcus kodakarensis, T. onnurineus, Sulfolobus solfataricus, S. islandicus, S. acidocaldarius, or Pyrococcus furiosus. The second temperature may be, for instance, at least 30° C. below the Tₒₚₜ of the genetically engineered archaeon, or 30° C. to 40° C. below the Tₒₚₜ of the genetically engineered archaeon.

In one embodiment, the promoter is a temperature sensitive promoter, and in such an embodiment expression of the coding region may be increased by at least 2-fold compared to expression of the coding region during growth at the first temperature. In one embodiment, the promoter is a constitutive promoter, and in one embodiment, the promoter is a heterologous promoter. In one embodiment, the promoter is an archaeal promoter. In one embodiment, the promoter is a bacterial promoter, and wherein the genetically engineered archaeon further includes a coding regions encoding a bacterial RNA polymerase that binds to the bacterial promoter and drives expression of the coding region operably linked to the bacterial promoter. In one embodiment, the coding regions encoding the bacterial RNA polymerase are operably linked to an archaeal promoter. In one embodiment, the genetically engineered archaeon further includes a cold repressed promoter operably linked to an endogenous coding region.

In one embodiment, the culturing includes culturing the genetically engineered archaeon at the first temperature until the genetically engineered archaeon reaches log phase or stationary phase. In one embodiment, the maintaining includes culturing the genetically engineered archaeon at the second temperature for at least 15 hours. In one embodiment, the method further includes shifting the culture after the maintaining back to the first temperature and culturing the genetically engineered archaeon at the first temperature. This culture can be further shifted to the to the second temperature, and the culture can be shifted from the first temperature to the second temperature and back again for multiple cycles.

In one embodiment, the polypeptide encoded by the coding region has an optimum activity at a temperature that is at least 20° C. below the Tₒₚₜ of the genetically engineered archaeon. In one embodiment, the genetically engineered archaeon includes more than one coding region operably linked to a promoter and present on the heterologous polynucleotide. In one embodiment, the genetically engineered archaeon includes more than one heterologous polynucleotide, wherein each heterologous polynucleotide includes at least one promoter operably linked to a coding region.

In one embodiment, the genetically engineered archaeon, such as Pyrococcus furiosus, includes a coding region encoding a polypeptide having acetyl/propionyl-CoA carboxylase activity, a coding region encoding a polypeptide having malonyl/succinyl-CoA reductase activity, and a coding region encoding a polypeptide having malonate semialdehyde, wherein each coding region is operably linked to a promoter. In one embodiment, the genetically engineered microbe includes NADPH-dependent hydrogenase activity.

Also provided herein is a cell-free method for using a genetically engineered archaeon. In one embodiment, the method includes providing a cell-free extract of a genetically engineered archaeon, wherein the genetically engineered archaeon includes a heterologous polynucleotide including a promoter operably linked to a coding region. The method further includes incubating the cell-free extract at a first temperature within 10° C. of optimum growth temperature (Tₒₚₜ) of the genetically engineered archaeon, and then incubating the cell-free extract at a second temperature that is at least 20° C. below the Tₒₚₜ of the genetically engineered archaeon. The extract is maintained at the second temperature, wherein activity of a polypeptide encoded by the coding region is increased compared to the activity of the polypeptide during incubation at the first temperature.

The cell-free extract may be produced from a genetically engineered archaeon that is, for instance, Thermococcus kodakarensis, T. onnurineus, Sulfolobus solfataricus, S. islandicus, S. acidocaldarius, or Pyrococcus furiosus. The second temperature may be, for instance, at least 30° C. below the Tₒₚₜ of the genetically engineered archaeon, or 30° C. to 40° C. below the Tₒₚₜ of the genetically engineered archaeon.

In one embodiment, the promoter is a temperature sensitive promoter, and in such an embodiment expression of the coding region may be increased by at least 2-fold compared to expression of the coding region during growth at the first temperature. In one embodiment, the promoter is a constitutive promoter, and in one embodiment, the promoter is a heterologous promoter. In one embodiment, the promoter is an archaeal promoter. In one embodiment, the promoter is a bacterial promoter, and wherein the cell-free extract further includes coding regions encoding a bacterial RNA polymerase that binds to the bacterial promoter and drives expression of the coding region operably linked to the bacterial promoter. In one embodiment, the coding regions encoding the bacterial RNA polymerase are operably linked to an archaeal promoter. In one embodiment, the cell-free extract further includes a cold repressed promoter operably linked to an endogenous coding region.

In one embodiment, the maintaining includes incubating the cell-free extract at the second temperature for at least 15 hours. In one embodiment, the method further includes shifting the cell-free extract after the maintaining back to the first temperature and incubating the cell-free extract at the first temperature. This extract can be further shifted to the second temperature, and the extract can be shifted from the first temperature to the second temperature and back again for multiple cycles.

In one embodiment, the polypeptide encoded by the coding region has an optimum activity at a temperature that is at least 20° C. below the Tₒₚₜ of the genetically engineered archaeon used to make the cell-free extract. In one embodiment, the cell-free extract includes more than one coding region operably linked to a promoter and present on the heterologous polynucleotide. In one embodiment, the cell-free extract includes more than one heterologous polynucleotide, wherein each heterologous polynucleotide includes at least one promoter operably linked to a coding region.

Also provided herein are genetically engineered archaea. A genetically engineered archaeon includes a heterologous polynucleotide. In one embodiment, the genetically engineered archaeon includes a promoter operably linked to a coding region, where the polypeptide encoded by the coding region has an optimum activity at a temperature that is at least 20° C. below the optimum growth temperature (Tₒₚₜ) of the genetically engineered archaeon. In one embodiment, the promoter is a constitutive promoter. In one embodiment, the promoter is a heterologous promoter. In one embodiment, the promoter is an archaeal promoter. In one embodiment, the promoter is a bacterial promoter, and the genetically engineered archaeon further includes coding regions encoding a bacterial RNA polymerase that binds to the bacterial promoter. In one embodiment, the coding regions encoding the bacterial RNA polymerase are operably linked to an archaeal promoter. In one embodiment, the genetically engineered archaeon further includes a cold repressed promoter operably linked to an endogenous coding region. In one embodiment, the genetically engineered archaeon includes more than one coding region operably linked to a promoter and present on the heterologous polynucleotide. In one embodiment, the genetically engineered archaeon includes more than one heterologous polynucleotide, where each heterologous polynucleotide includes at least one promoter operably linked to a coding region.

As used herein, a “hyperthermophile” is a member of the domain Archaea that thrives in environments of at least 75° C. A member of the domain Archaea may be referred to herein as archaea (plural) or archaeon (singular). Depending upon the context, the term “microbe” may also refer to a member of the domain Archaea.

As used herein, a “thermophile” is a member of the domain Bacteria or Archaea that thrives in environments between 50° C. and no greater than 75° C.

As used herein, a “microbe” is a single celled organism that is a member of the domain Archaea or a member of the domain Bacteria.

As used herein, “optimum growth temperature” and “Tₒₚₜ” refer to the optimal growth temperature of a microbe. The optimal growth temperature of a microbe is the temperature at which the doubling time is the shortest. The Tₒₚₜ of a thermophilic archaeon is between 50° C. and no greater than 75° C., and the Tₒₚₜ of a hyperthermophilic archaeon is between 75° C. and up to 100° C.

As used herein, “genetically engineered archaeon” refers to an archaeon, either hyperthermophilic or thermophilic, which has been altered “by the hand of man,” for instance, by the introduction of a heterologous polynucleotide. For example, an archaeon is a genetically engineered archaeon by virtue of introduction into a suitable archaeon of a heterologous polynucleotide. “Genetically engineered archaeon” also refers to an archaeon that has been genetically manipulated such that endogenous nucleotides have been altered. For example, an archaeon is a genetically engineered archaeon by virtue of introduction into a suitable archaeon of an alteration of endogenous nucleotides. For instance, an endogenous coding region could be deleted or mutagenized. Such mutations may result in a polypeptide having a different amino acid sequence than was encoded by the endogenous polynucleotide. Another example of a genetically engineered archaeon is one having an altered regulatory sequence, such as a promoter, to result in altered expression of an operably linked endogenous coding region.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

An “heterologous polynucleotide” refers to a foreign polynucleotide, i.e., a polynucleotide that is not normally present in an archaeon. A heterologous polynucleotide may be separate from the genomic DNA of a cell (e.g., it may be a vector, such as a plasmid), or a heterologous polynucleotide may be integrated into the genomic DNA of a cell. A regulatory region, such as a promoter, that is present in the genomic DNA of an archaeon but has been modified to have a nucleotide sequence that is different from the promoter normally present in the archaeon is also considered a heterologous polynucleotide. A heterologous polynucleotide may encode a heterologous polypeptide or an endogenous polypeptide.

A “coding region” is a nucleotide sequence that encodes a polypeptide, and when placed under the control of appropriate regulatory sequences expresses the encoded polypeptide. The boundaries of a coding region are generally determined by a translation start codon at its 5′ end and a translation stop codon at its 3′ end. A regulatory sequence is a nucleotide sequence that regulates expression of a coding region to which it is operably linked. Nonlimiting examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

As used herein, the term “polypeptide” refers broadly to a polymer of two or more amino acids joined together by peptide bonds. The term “polypeptide” also includes molecules which contain more than one polypeptide joined by disulfide bonds, ionic bonds, or hydrophobic interactions, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

An “heterologous polypeptide” refers to a foreign polypeptide, i.e., a polypeptide that is not normally present in an archaeon. An “endogenous polypeptide” refers to a polypeptide that is normally present in an archaeon. Since a heterologous polynucleotide may include, in some embodiments, a polynucleotide that is normally present in a microbe but is operably linked to a regulatory region to which it is not normally operably linked, in some embodiments a heterologous polynucleotide may encode an endogenous polypeptide.

As used herein, the “optimal activity” of a polypeptide refers temperature under which the rate of a reaction catalyzed by the polypeptide is at its highest.

As used herein, “identity” refers to structural similarity between two polypeptides or two polynucleotides. The structural similarity between two polypeptides is determined by aligning the residues of the two polypeptides (e.g., a candidate amino acid sequence and a reference amino acid sequence) to optimize the number of identical amino acids along the lengths of their sequences; gaps in either or both sequences are permitted in making the alignment in order to optimize the number of shared amino acids, although the amino acids in each sequence must nonetheless remain in their proper order. The structural similarity is typically at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity. A candidate amino acid sequence can be isolated from a microbe, such as, but not limited to, a Pyrococcus spp., including P. furiosus, or a Metallosphaera spp., including M. sedula, or can be produced using recombinant techniques, or chemically or enzymatically synthesized. Structural similarity may be determined, for example, using sequence techniques such as the BESTFIT algorithm in the GCG package (Madison Wis.), or the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix=BLOSUM62, gap costs=existence: 11 extension: 1, compositional adjustments=conditional compositional score matrix adjustment. Alternatively, polypeptides may be compared using the BESTFIT algorithm in the GCG package (version 10.2, Madison Wis.). In the comparison of two amino acid sequences using the BLAST search algorithm, structural similarity is referred to as “identities.”

As used herein, an “isolated” substance is one that has been removed from its natural environment, produced using recombinant techniques, or chemically or enzymatically synthesized. For instance, a polypeptide, a polynucleotide, or a product produced using a method described herein can be isolated. Preferably, a substance is purified, i.e., is at least 60% free, preferably at least 75% free, and most preferably at least 90% free from other components with which it is naturally associated.

Conditions that are “suitable” for an event to occur, such as expression of a coding region or production of a product, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Recombinant expression of lactate dehydrogenase (LDH) in P. furiosus strain LAC changes its fermentation pattern. (A) Concept of temperature-dependent switch in end product formation by P. furiosus. Abbreviations: GAPOR, glyceraldehyde-3-phosphate ferredoxin oxidoreductase; POR, pyruvate ferredoxin oxidoreductase; Fd, ferredoxin; acetyl-CoA, acetyl coenzyme A; Cbes LDH, C. bescii LDH. (B) Specific activity of lactate dehydrogenase in the protein extract of C. bescii DSM 6725, P. furiosus DSM 3638 (wild type), P. furiosus ΔpdaD host strain, and P. furiosus LAC obtained from 400-ml batch cultures. (C) Lactate production in the same P. furiosus cultures. Values given are averages±standard deviations (SD) (error bars) of three independent biological cultures.

FIG. 2. Plasmid vector pMPF301 containing the pdaD P_{cipA}Cbes-ldh cassette, 1-kb upstream and downstream flanking regions of the pdaD gene and the apr gene as a selective marker in Escherichia coli (apramycin resistance). Plasmid diagrams were constructed using Vector NTI software (Invitrogen).

FIG. 3. Cloning strategy for the mutant strain P. furiosus LAC. The fusion product P_{cipA}Cbes-ldh was obtained by overlapping PCR and integrated into vector pSPF300 (Hopkins et al., 2011. PLoS One 6:e26569). The new vector, pMPF301, additionally carried the pdaD gene essential for agmatine biosynthesis and 1-kb upstream and downstream flanking regions of the pdaD gene. Linearized DNA was used for transformation of the P. furiosus ΔpdaD host strain. The pdaD P_{cipA}Cbes-ldh cassette integrated into the genome by homologous recombination, replacing the P_{gdh}pyrF cassette. Therefore, the resulting new strain, P. furiosus LAC, exhibits a uracil auxotrophy, but does not, in contrast to the host, require agmatine for growth.

FIG. 4. (A and B) Lactate production (squares), acetate production (triangles), cell density (circles), and relative mRNA fold expression levels (broken lines) in 15-liter fermentor cultures of P. furiosus LAC. One culture was grown at 72° C. (A), while another culture was grown at 94° C. and rapidly cooled to 72° C. after a cell density of 1.5 10⁸ was reached (indicated by the black arrow) (B). After the temperature switch, higher mRNA levels for the heterologous gene Cbes-ldh, high specific activity of lactate dehydrogenase, and a high rate of lactate formation were observed.

FIG. 5. Recombinant expression and activity of C. bescii lactate dehydrogenase in P. furiosus at different temperatures. (A and B) Cell density (A) and relative Cbes-ldh mRNA level and specific activity of lactate dehydrogenase (LDH) (B) in cell extracts of P. furiosus LAC grown at different temperatures (for 72° C., n=2; for 98° C., n=1). Although growth was negligible at 72° C. and 75° C., the highest ldh mRNA level and lactate dehydrogenase activities were observed at these growth temperatures. (C and D) Thermo stability (C) and temperature dependence of lactate dehydrogenase activity (D) in protein extracts of C. bescii DSM 6725 (native LDH) and P. furiosus strain LAC (recombinant LDH) grown at 75° C. and harvested in the stationary phase. Values given are averages±SD of three independent biological cultures (B) or three independent enzymatic measurements (D), unless denoted otherwise. n.d., not determined.

FIG. 6. 4-hydroxybutryate-CoA ligase candidates in M. sedula. Normalized transcription levels for M. sedula genes annotated as small organic acid or fatty-acid ligases and synthetases. High transcription levels are shown in red, low transcription in green, corresponding numbers represent leastsquares means of normalized log 2-transformed transcription levels relative to the overall average transcription level of 0 (black). Conditions shown: (2010)—Heterotrophic, Autotrophic, Mixotrophic; (2012)—Autotrophic Carbon Limited (ACL), Autotrophic Carbon Rich (ACR), Heterotrophic (HTR).

FIG. 7. Specific activity of acyl-CoA ligases in the M. sedula carbon fixation pathway. Specific activities of the new candidates for 4-hydroxybutyrate-CoA ligase on a variety of substrates compared to reported data for Msed_{—}1456, a 3-hydroxypropionate-CoA ligase: Msed_{—}0394 (A), Msed_{—}0406 (B), and Msed_{—}1456 (C). Msed_{—}1456 showed >1% activity on 3-hydroxybutyrate, but was not tested on 4-hydroxybutyrate. Substrate abbreviations: ACE—acetate; PRO—propionate; 3HP—3-hydroxypropionate; 4HB—4-hydroxybutyrate; BUT—butyrate; VAL—valerate.

FIG. 8. Specific activity of native Msed_{—}1353 and Msed_{—}1353-W424G mutant. Comparison of activity of Msed_{—}1353 (A) and Msed_{—}1353-G424 (B) on a variety of small organic acids. Substrate abbreviations: ACE—acetate; PRO—propionate; 3HP—3-hydroxypropionate; 4HB—4-hydroxybutyrate; BUT—butyrate; VAL—valerate; HEX—hexanoate; OCT—octanoate.

FIG. 9. S. enterica acetyl-CoA synthetase (Acs) and Msed_{—}0394 active site comparison. Acs shown in gold (residue W414), Msed_{—}0394 in cyan (residues W233, L307, V331, and P340). Ligand from Acs structure (adenosine-5′-propyl phosphate) is labeled Acs. A) Side view of binding pocket with inter-atomic distances given from phosphorus atom of propyl-phosphate moiety to select atom from amino acid residues. B) Axial view from bottom of substrate binding pocket.

FIG. 10. Sequence alignment of S. enterica acetyl-CoA synthetase (STM4275) and M. sedula acyl-CoA ligases. Amino acid sequence alignment of active site residues in putative acyl-CoA ligases reveals a conserved glycine (shown in box) except for Msed_{—}1353, which has a tryptophan indicative of acetate-propionate CoA ligases. Alignment was generated using Chimera by superposition of I-TASSER 3D structural models.

FIG. 11. (A) The synthetic operon constructed to express the M. sedula genes encoding E1 (αβγ), E2 and E3 in P. furiosus under the control of the promoter for the S-layer protein gene (Pslp). This includes P. furiosus ribosomal binding sites (rbs) from highly-expressed genes encoding pyruvate ferredoxin oxidoreductase subunit γ (pory, PF0971), the S-layer protein (slp, PF1399) and cold-induced protein A (cipA, PF0190). (B) The first three enzymes of the M. sedula 3-HP/4-HB cycle produce the key intermediate 3-hydroxypropionate (3-HP). E1 is acetyl/propionyl-CoA carboxylase (αβγ, encoded by Msed_{—}0147, Msed_{—}0148, Msed_{—}1375): E2 is malonyl/succinyl-CoA reductase (Msed_{—}0709) and E3 is malonate semialdehyde reductase (Msed_{—}1993). NADPH is generated by P. furiosus soluble hydrogenase I (SH1), which reduces NADP with hydrogen gas. (C) The first three enzymes (E1-E3) are shown in context of the complete 3-HP/4-HP cycle for carbon dioxide fixation by Metallosphaera sedula showing the three subpathways, SP1 (blue), SP2 (green) and SP3 (red). (D) The horizontal scheme shows the amount of energy (ATP), reductant (NADPH), oxidant (NAD) and CoASH required to generate one mole of acetyl-CoA from two moles of carbon dioxide.

FIG. 12. Plasmid map of pALM506-1 used to transform P. furiosus strain ΔpdaD to generate strain PF506.

FIG. 13. Plasmid map of pGL007 vector targeting the region between PF0574 and PF0575 in the P. furiosus genome.

FIG. 14. Plasmid map of pGL010 used to transform P. furiosus COM1 to generate strain MW56.

FIG. 15. Temperature-dependent production of the SP1 pathway enzymes in P. furiosus strain PF506. (A) Growth at 98° C. (circles) and temperature (black line) for the temperature shift from 98 to 75° C. are shown. (B) Activities of E1, E2+E3, and E1+E2+E3 after the temperature shift to 75° C. for the indicated time period (see FIG. 18). The activities of a cell-free extract of autotrophically-grown M. sedula cells is also shown (labeled Msed). The specific activities are: E1+E2+E3 coupled assay with acetyl-CoA and bicarbonate (first column of each group of three columns at each time), E2+E3 coupled assay with malonyl-CoA (second column of each group of three columns at each time), and E2 with succinyl-CoA (third column of each group of three columns at each time) as substrates. (C) Specific activity (μmoles NADPH oxidized/min/mg) of the coupled activity of E2+E3 in cell-free extracts after cell growth at 95° C. to a high cell density of 1×10⁸ cells/ml followed by incubation for 18 hrs at the indicated temperature. (D) Temperature dependence of the coupled activity of E2+E3 (circles) in the cell-free extracts after induction at 72° C. for 16 hr. The activity of P. furiosus glutamate dehydrogenase in the same cell-free extracts is also shown (squares).

FIG. 16. Growth of P. furiosus strain PF506 at 98° C. and subsequent temperature shift to 75° C. P. furiosus was grown in four 800 mL cultures at 98° C. until the cell density reached 5×10⁸ cells/mL. The temperature (shown as black line) was then shifted to 75° C. and individual bottles were removed and harvested after 0 (cross), 16 (triangle), 32 (diamond) and 48 (square) hrs. The enzyme activities in each cell type are summarized in FIG. 17.

FIG. 17. Stability of E2 and E3 using an E2+E3 coupled assay at 75° C. after incubation at 90° C. for the indicated amount of time in cell-free extracts of P. furiosus strain PF506 (circles) and of the endogenous P. furiosus glutamate dehydrogenase (squares). The specific activity of E2+E3 in PF506 (grown at 72° C.) is about 2-fold higher than that measured in M. sedula. Activity is expressed as percent of maximum activity.

FIG. 18. Growth of P. furiosus COM 1, MW56 and PF506 during the temperature shift from 98° C. to 70° C. Cell densities of COM1 (diamonds), MW0056 (squares), and PF506 (triangles) are indicated. The 400 mL cultures were grown at 95° C. for 9 hr and then allowed to cool at room temperature to 70° C. before being placed in a 70° C. incubator.

FIG. 19. Enzyme activities of E1 (first column of each pair) and coupled E2+E3 (second column of each pair) in cell-free extracts of the indicated P. furiosus strains after incubation at 70° C. for 18 hrs, compared to that measured for the cell-extract of autotrophically-grown M. sedula cells (labeled Msed).

FIG. 20. ESI-MS identification of 3-HP produced from acetyl-CoA, CO2 and H2 (or NADPH) by cell-free extracts of P. furiosus strains ΔPdaD (A) and PF506 (B). The MS peak corresponding to the 3HP derivative (m/z 224, circle) was present above background only in the recombinant PF506 strain.

FIG. 21. 3-HP production by P. furiosus. Cells were grown at 95° C. and then incubated at 72° C. for 16 hr to produce the SP1 enzymes. (A) In vitro 3-HP production from acetyl-CoA. The sources of the C1 carbon (CO₂ or HCO₃—) and reducing equivalents (NADPH or NADP/H₂) are indicated. Rates are expressed as μmoles of 3-HP produced/min/mg. (B) In vivo 3-HP production by whole cells using maltose as the source of acetyl-CoA in the presence of hydrogen gas and bicarbonate. The P. furiosus strains are MW56 (circles) and COM1 (squares).

FIG. 22. Maltose and pyruvate metabolism by P. furiosus, and the key roles of pyruvate ferredoxin oxidoreductase (POR) in acetyl-CoA production and of the membrane-bound hydrogenase (MBH) in H₂ production.

FIG. 23. Design of an artificial operon encoding SP1 (E1-E3) for expression in P. furiosus.

FIG. 24. SP1 expression cassette for cloning into pSPF300 vector (SEQ ID NO:75).

FIG. 25. Construction of pALM506-1 plasmid for transformation of P. furiosus strain ΔpdaD (SEQ ID NO:76).

FIG. 26. Transcriptionally inactive zones for foreign gene insertion.

FIG. 27. Target genome regions in NCBI reference sequence versus COM1 sequence.

FIG. 28. SOE-PCR products for constructing pGL002 (SEQ ID NO:77) and pGL007 (SEQ ID NO:78) targeting genome regions 2 and 3.

FIG. 29. Construction of pGL002 vector targeting genome region 2.

FIG. 30. Construction of pGL007 vector targeting genome region 3.

FIG. 31. SP2B expression cassette for cloning into pGL002 (SEQ ID NO:79).

FIG. 32. Construction of pGL005 vector for transformation of P. furiosus COM1.

FIG. 33. SP1 expression cassette for cloning into pGL007 (SEQ ID NO:80).

FIG. 34. Construction of pGL010 vector for transformation of COM1.

FIG. 35. NADPH-dependent assays for the E2, E2+E3 and E1+E2+E3 reactions of SP 1.

FIG. 36. NADPH-dependent assay for E9 of the SP2B subpathway.

FIG. 37. Growth of P. furiosus strain MW43 at 95° C. and temperature shift from 65° C. to 90° C. for 18 hrs.

FIG. 38. E9 temperature profile and stability in cell-free extracts of P. furiosus strain MW43. A) E9 specific activity in MW43 versus Msed extract. B) E9 specific activity when assayed at increasing temperatures. C) Stability of E9 over time when incubated at 90° C.

FIG. 39. Phosphate-dependent assay for E1.

FIG. 40. Scheme for producing acetyl CoA from pyruvate or maltose and for producing ATP and NADPH for the SP1 pathway for 3-HP production by whole cells of P. furiosus strains PF506 and MW56.

FIG. 41. Promoter region of PF0882 of P. furiosus (SEQ ID NO:1), promoter region of PF0421 of P. furiosus (SEQ ID NO:2), and promoter region of PF0198 of P. furiosus (SEQ ID NO:3).

FIG. 42. Bacterial promoter/RNA polymerase combinations.

FIG. 43. Amino acid sequences of polypeptides that are part of the 4-hydroxybutyrate cycle.

### DETAILED DESCRIPTION OF ILLUSTRATIVE EMBODIMENTS

The present invention provides genetically engineered microbes that are members of the domain Archaea, and methods for expressing polypeptides in such genetically engineered microbes. Useful archaea include those having a genetic system that allows the introduction of DNA into a cell. Examples of useful hyperthermophilic archaea include, but are not limited to, Thermococcus kodakarensis, T. onnurineus, Sulfolobus solfataricus, S. islandicus, S. acidocaldarius, and Pyrococcus furiosus. It is expected that genetic systems will be established in other members of the domain Archaea, both hyperthermophilic and thermophilic, and that it will be possible to use such archaea in the methods described herein.

Thermococcus kodakarensis, T. onnurineus, Sulfolobus solfataricus, S. islandicus, S. acidocaldarius, and P. furiosus that can be genetically manipulated are readily available. For instance, these Archaea may be obtained from their natural environment using methods known in the art. In one embodiment, an example of a Thermococcus kodakarensis that can be used in the methods described herein is described in Sato et al. (2003, J. Bacteriol., 185:210-220). In one embodiment, an example of a T. onnurineus that can be used in the methods described herein is KDO1, which is described in Sato et al. (2003, J. Bacteriol., 185:210-220). In one embodiment, an example of a Sulfolobus solfataricus that can be used in the methods described herein is described in Worthington et al., (2003, J. Bacteriol., 185:482-488). In one embodiment, an example of a S. islandicus that can be used in the methods described herein is described in Deng et al., (2009, Extremophiles, 13:735-746). In one embodiment, an example of a S. acidocaldarius that can be used in the methods described herein is described in Wagner et al., (2009, Biochem. Soc. Trans., 37:97-101). In one embodiment, the P. furiosus is COM1 (Lipscomb et al., 2011, Appl. Environ. Microbiol., 77:2232-2238; Lipscomb et al., U.S. Published Patent Application 20120135411), and deposited with American Type Culture Collection (ATCC), American Type Culture Collection (ATCC), 10801 University Boulevard, Manassas, Va., 20110-2209, USA, on Sep. 14, 2010. This deposit, designated PTA-11303, will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

An archaeon used herein is genetically engineered to include a heterologous polynucleotide. In one embodiment, a heterologous polynucleotide includes a promoter, and the promoter may be heterologous or endogenous. A promoter acts as a regulatory signal that binds an RNA polymerase to initiate transcription of an operably linked coding region. The promoter is operably linked to a coding region, and the coding region may encode a heterologous polypeptide or an endogenous polypeptide. In one embodiment, a promoter is operably linked to more than one coding region, encoding heterologous polypeptides, endogenous polypeptides, or a combination thereof. Such an arrangement of one promoter controlling expression of two or more operably linked coding regions is often referred to as an operon. In one embodiment, a heterologous promoter may be present in the genomic DNA and operably linked to an endogenous coding region. The present invention also includes a genetically engineered archaeon.

In one embodiment, a promoter is one that functions in an archaeon, e.g., a promoter that is recognized by a highly conserved transcription complex present in archaea cells. Such a promoter may be referred to herein as an archaeal promoter. Archaeal promoters do not have the same structure as promoters present in members of the domain Bacteria. One transcription factor important in the transcription of archaeal coding regions is TFB, a homologue of the eukaryotic TFIIB. Archaeal promoters often include a TATA box which may be centered 24 to 28 nucleotides upstream of a transcription start site, and the TATA box can be represented as a conserved 8 base pair sequence element TTTAWAta (SEQ ID NO:1), where W is A or T, and R is A or G. An archaeal promoter may also include a TFB responsive element (cRNaANt, SEQ ID NO:2, where R is A or G, and N is any nucleotide) upstream and adjacent to the TATA box (Gregor and Pfeifer, 2005, Microbiology, 151:25-33; Bell et al., 1999, Mol. Cell., 4:971-982; Bell et al., 1999, PNAS USA, 96:13662-13667).

The promoter useful in the methods described herein may be, but is not limited to, a constitutive promoter, a temperature sensitive promoter, a non-regulated promoter, or an inducible promoter. A constitutive promoter drives expression of an operably linked coding region in an archaeon when cultured at the temperatures described herein. The expression of a coding region operably linked to a constitutive promoter occurs at both high and low incubation temperatures, and the level of expression does not change substantially when expression at higher and lower incubation temperatures is compared. An example of a constitutive promoter is Pₛₗₚ, a P. furiosus promoter of the highly expressed S-layer protein (Chandrayan et al., 2012. J. Biol. Chem., 287:3257-3264). Other examples of constitutive promoters include P_{gdh}, Pₚₑₚ and P_{pory}, which are promoters in both P. furiosus and T. kodakarensis of the highly expressed glutamate dehydrogenase, phosphoenolpyruvate synthase and pyruvate ferredoxin oxidoredutase subunit γ, respectively (for example, see Lipscomb et al. 2011. Appl. Environ. Microbiol. 77:2232-2238; Chandrayan et al., 2012. J. Biol. Chem., 287:3257-3264).

The promoter may be a temperature sensitive promoter. In one embodiment, a temperature sensitive promoter drives expression of an operably linked coding region in an archaeon at a greater level during incubation at low temperatures when compared to expression during incubation at high temperature. Such a promoter is referred to herein as a “cold shock” promoter. A cold shock promoter is induced at temperatures lower than the Tₒₚₜ of an archaeon. In one embodiment, a cold shock promoter is induced when an archaeon is cultured at a temperature of no greater than 75° C., no greater than 70° C., no greater than 65° C., no greater than 60° C., no greater than 55° C., no greater than 50° C., no greater than 45° C., no greater than 40° C., or no greater than 35° C. In one embodiment, a cold shock promoter is induced when an archaeon is cultured at a temperature between 35° C. and 45° C., between 40° C. and 50° C., between 45° C. and 55° C., between 50° C. and 60° C., between 55° C. and 65° C., between 60° C. and 70° C., or between 65° C. and 75° C. Induction of a cold shock promoter in a genetically engineered archaeon may result in an upregulation of expression of an operably linked coding region by at least 10-fold, at least 15-fold, at least 20-fold, at least 25-fold, or at least 30-fold compared to expression of the same operably linked coding region during growth of the genetically engineered archaeon at its Tₒₚₜ.

Examples of cold shock promoters include those operably linked to the coding regions of P. furiosus described by Weinberg et al., (2005, J. Bacteriol., 187:336-348). A promoter is present in the region immediately upstream of the first codon of a coding region. In one embodiment, at least 150 nucleotides upstream to at least 200 nucleotides upstream of the first codon of the operably linked coding region includes the promoter. The size of the region that includes a promoter may be limited by the presence of an upstream coding region such as a start codon (for a coding region on the opposite strand) or a stop codon (for a coding region on the same strand). Identifying promoters in microbes, including hyperthermophilic archaeae and thermophilic archaeae, is routine (see, for example, Lipscomb et al., 2009, Mol. Microbiol., 71:332-349). Other archaea contain homologues of the coding regions described by Weinberg et al., and the promoters of such homologues can be evaluated for induced expression at lower temperatures. Cold sock promoters may be produced using recombinant techniques.

In one embodiment, a temperature sensitive promoter drives expression of an operably linked coding region in an archaeon at a decreased level during incubation at low temperatures when compared to expression during incubation at high temperature. Such a promoter is referred to herein as a “cold repressed” promoter. As described herein, a genetically engineered archaeon may be used to produce a product; however, the archaeon may normally produce an endogenous enzyme that uses the product or an intermediate leading to the product. The use of a cold repressed promoter is advantageous in such an embodiment. The genetically engineered archaeon may be modified to decrease the production of the endogenous enzyme. For instance, an archaeon may be genetically engineered by removing the promoter driving expression of an endogenous enzyme and replacing it with a cold repressed promoter.

A cold repressed promoter is repressed at temperatures lower than the Tₒₚₜ of an archaeon. In one embodiment, a cold repressed promoter is repressed when an archaeon is cultured at a temperature of no greater than 75° C., no greater than 70° C., no greater than 65° C., no greater than 60° C., no greater than 55° C., no greater than 50° C., no greater than 45° C., no greater than 40° C., or no greater than 35° C. In one embodiment, a cold repressed promoter is induced when an archaeon is cultured at a temperature between 35° C. and 45° C., between 40° C. and 50° C., between 45° C. and 55° C., between 50° C. and 60° C., between 55° C. and 65° C., between 60° C. and 70° C., or between 65° C. and 75° C. The use of a cold repressed promoter in a genetically engineered archaeon may result in an down-regulation of expression of an operably linked coding region by at least 10-fold, at least 15-fold, at least 20-fold, at least 25-fold, or at least 30-fold compared to expression of the same operably linked coding region during growth of the genetically engineered archaeon at its Tₒₚₜ.

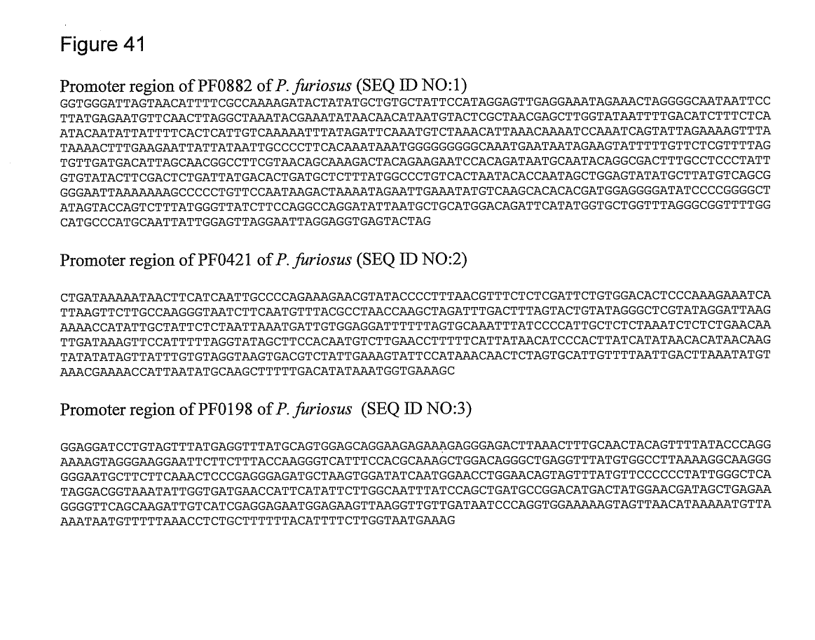

Cold repressed promoters present in hyperthermophilic archaea and thermophilic archaea can be easily identified using routine methods. For instance, DNA microarray analysis can be used to compare expression of coding regions in an archaeon, such as a hyperthermophile, grown at its Tₒₚₜ and the archaeon hyperthermophile grown at a temperature below the Tₒₚₜ. The temperature below the Tₒₚₜ may be, for instance, at least 20° C., at least 30° C., at least 40° C. below the Tₒₚₜ. The decrease in expression may be a change of at least 5-fold, at least 10-fold, at least 15-fold, or at least 20-fold when comparing expression at the two temperatures. Examples of cold repressed promoters include, but are not limited to, the promoter upstream of the hypothetical polypeptide encoded by coding region PF0882 of P. furiosus (SEQ ID NO:1), the promoter upstream of the polypeptide encoded by coding region PF0421 of P. furiosus (SEQ ID NO:2), and the promoter upstream of the polypeptide encoded by coding region PF0198 of P. furiosus (SEQ ID NO:3) (FIG. 41). The promoters disclosed in FIG. 41 may be used by attaching a coding region such that the first codon of the coding region is present immediately adjacent to and downstream of the nucleotide located at the 3′ end. In one embodiment, a promoter disclosed in FIG. 41 includes at least 200 consecutive nucleotides, at least 250 consecutive nucleotides, at least 300 consecutive nucleotides, at least 350 consecutive nucleotides, or at least 400 consecutive nucleotides selected from the polynucleotide disclosed in FIG. 41.

The heterologous polynucleotide that is present in a genetically engineered archaeon may include other regulatory elements, in addition to a promoter, that are operably linked to a coding region. Such regulatory elements may be chosen to optimize expression of an operably linked coding region, and include, for instance, a ribosomal binding site to optimize translation of an operably linked coding region. In one embodiment, regulatory elements may be chosen from, or based on, the same genus of archaeon as the genetically engineered archaeon. For instance, if the genetically engineered archaeon is P. furiosus, regulatory elements included with the heterologous polynucleotide can be based on those present in P. furiosus.

In one embodiment, a promoter that is part of a heterologous polynucleotide present in a genetically engineered archaeon is one that functions in a member of the domain Bacteria. Such a promoter is also referred to as a bacterial promoter. The characteristics of bacterial promoters are known to the person skilled in the art, and include, for instance, a −10 element and a −35 element. A consensus sequence for the −10 element is TATAAT, and a consensus sequence for the −35 element is TTGACA; however, these consensus sequences are often not present in a promoter. Instead, a −10 element and a −35 element of a bacterial promoter often has only three or four of the six nucleotides in an element that match the consensus. Some bacterial promoters may also include an UP element, located upstream of the −35 element. Bacterial promoters are recognized by bacterial RNA polymerase, and are not recognized by a native RNA polymerase normally produced by an archaeon. Bacterial RNA polymerase includes 5 subunits, including a sigma subunit. Bacterial promoters having a −10 element and a −35 element as described above are recognized by an RNA polymerase that includes a sigma-70 subunit.

A bacterial promoter present in a genetically engineered archaeon requires a bacterial RNA polymerase to drive expression of a coding region operably linked to the bacterial promoter. Thus, a genetically engineered archaeon containing a bacterial promoter on a heterologous polynucleotide also includes coding regions encoding the subunits of an RNA polymerase that will recognize and bind to a bacterial promoter and result in expression of a coding region operably linked to the bacterial promoter. A bacterial promoter and the coding regions encoding the RNA polymerase subunits may be on the same heterologous polynucleotide or may be on separate heterologous polynucleotides in a genetically engineered archaeon. Coding regions encoding RNA polymerase subunits present on a heterologous polynucleotide present in a genetically engineered archaeon are operably linked to a promoter described herein, such as a temperature sensitive promoter or a constitutive promoter that functions in an archaeon.

In one embodiment, a genetically engineered archaeon may include a bacterial promoter operably linked to a coding region encoding a polypeptide of interest. The genetically engineered archaeon will also include coding regions encoding RNA polymerase subunits that will bind to and turn on the bacterial promoter. When the coding regions encoding RNA polymerase subunits are operably linked to a promoter that functions in an archaeon, the archaeon will produce the RNA polymerase subunits and the RNA subunits will bind to the bacterial promoter and drive expression of the operably linked coding region.

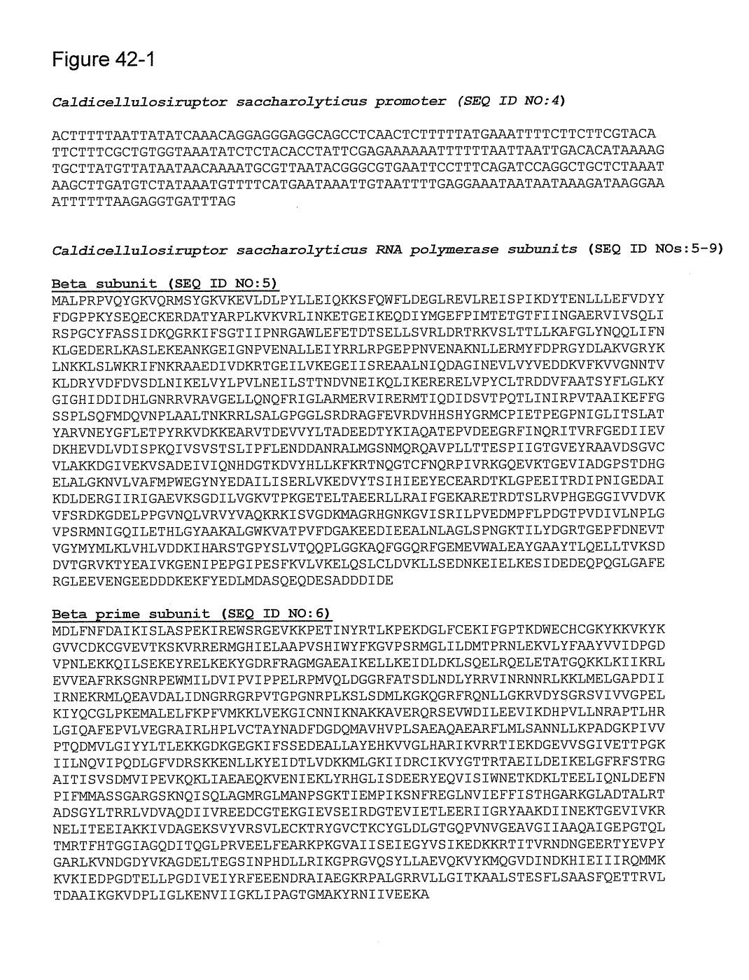

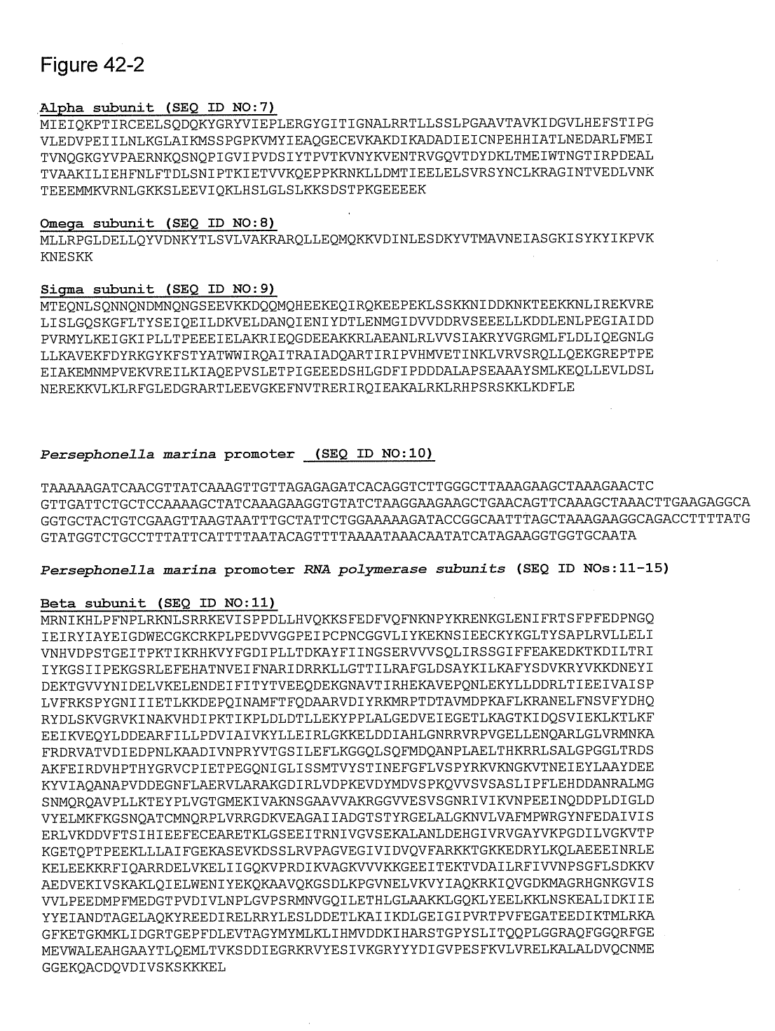

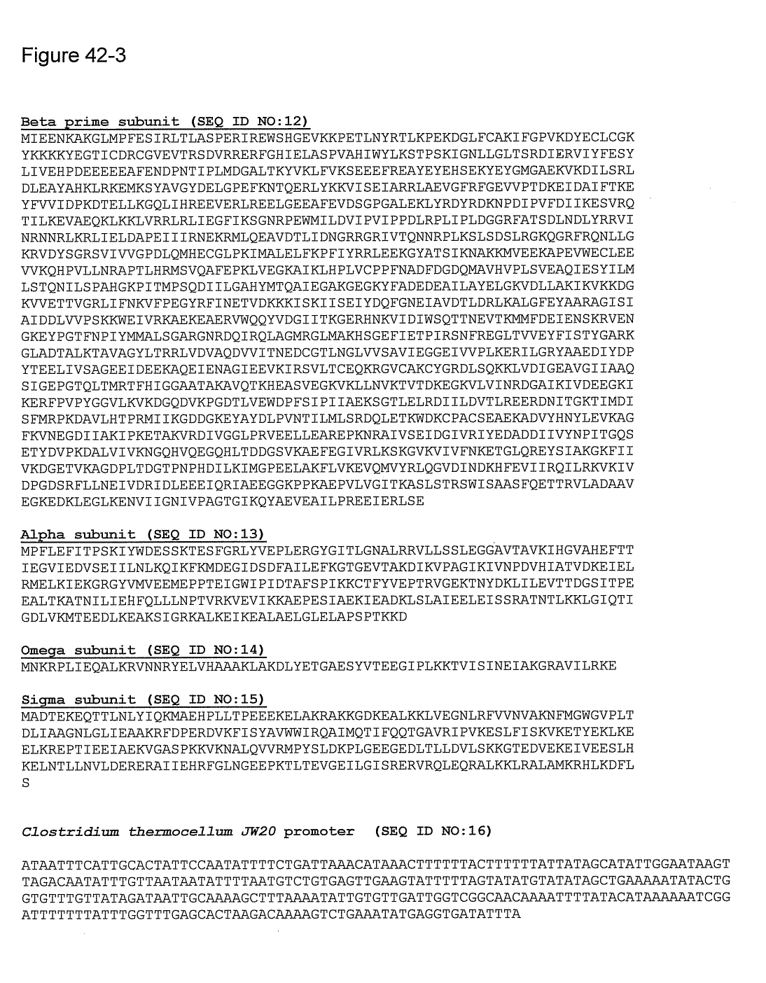

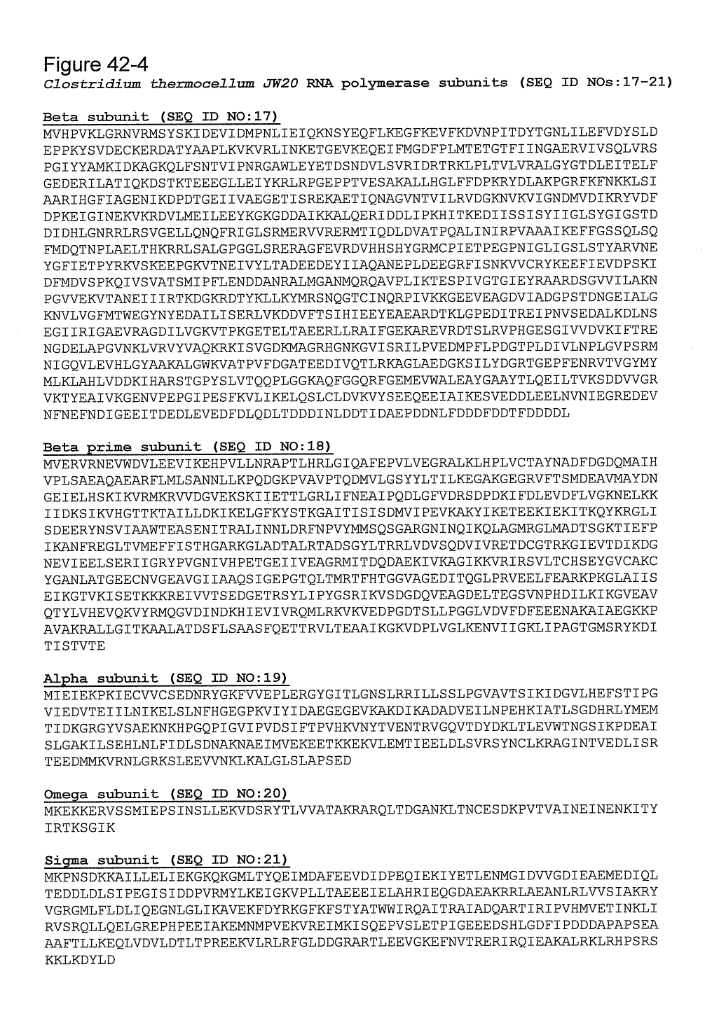

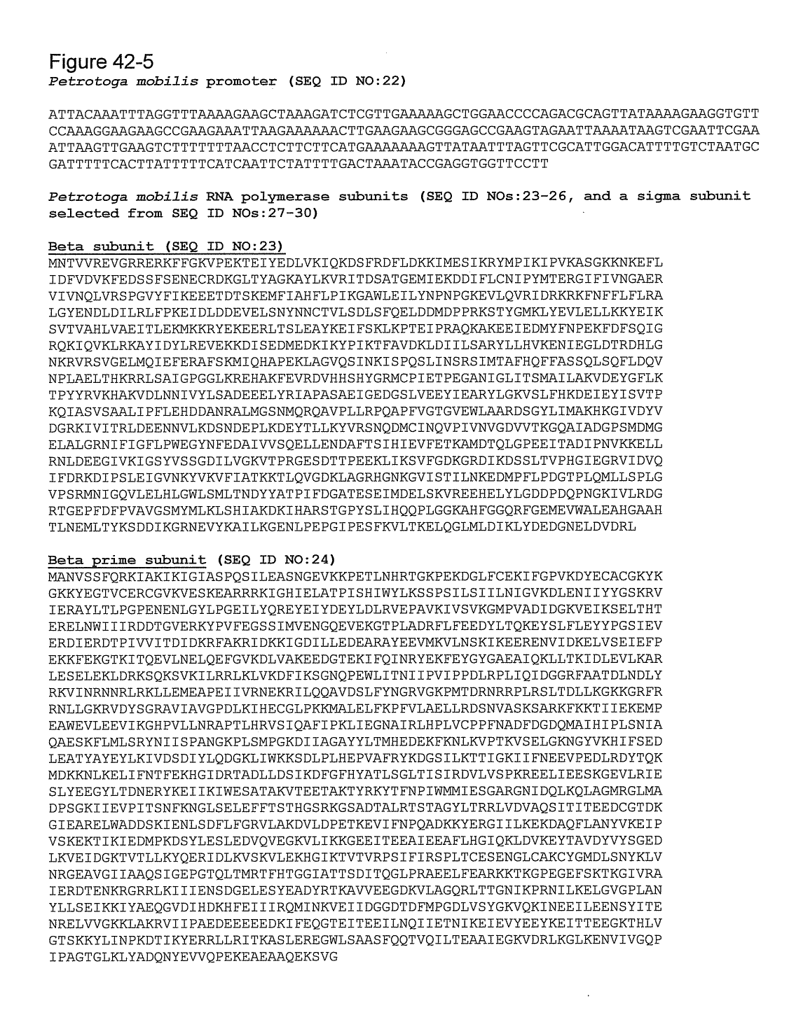

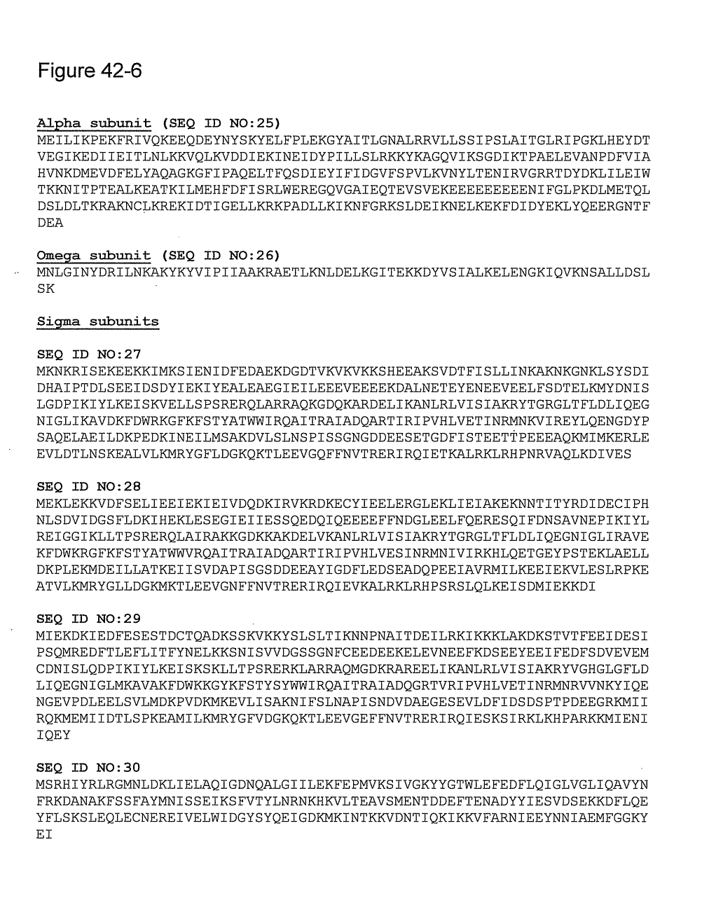

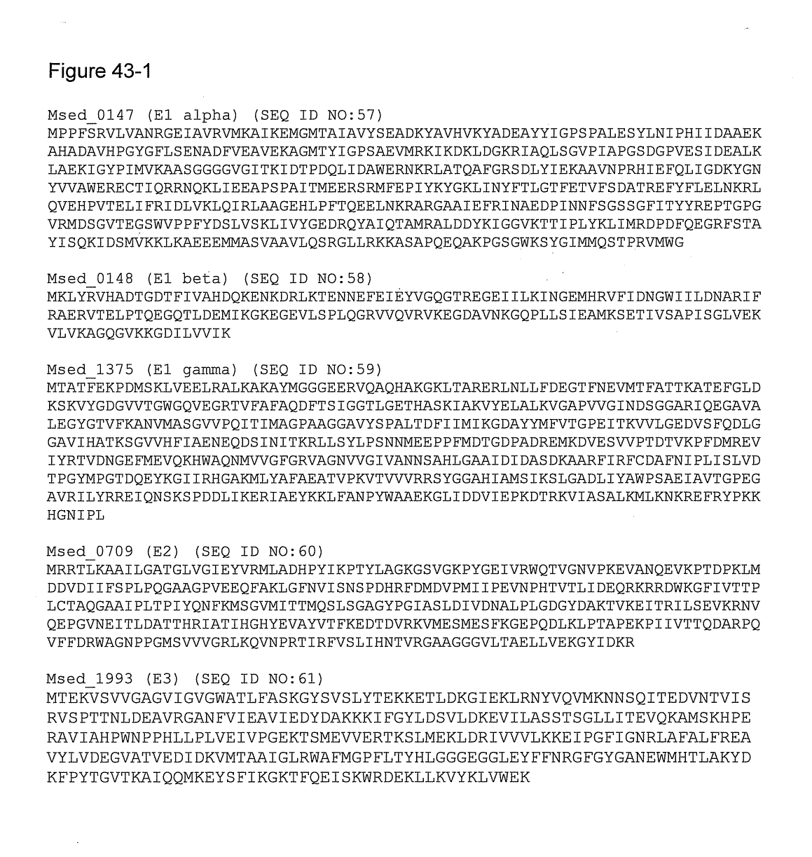

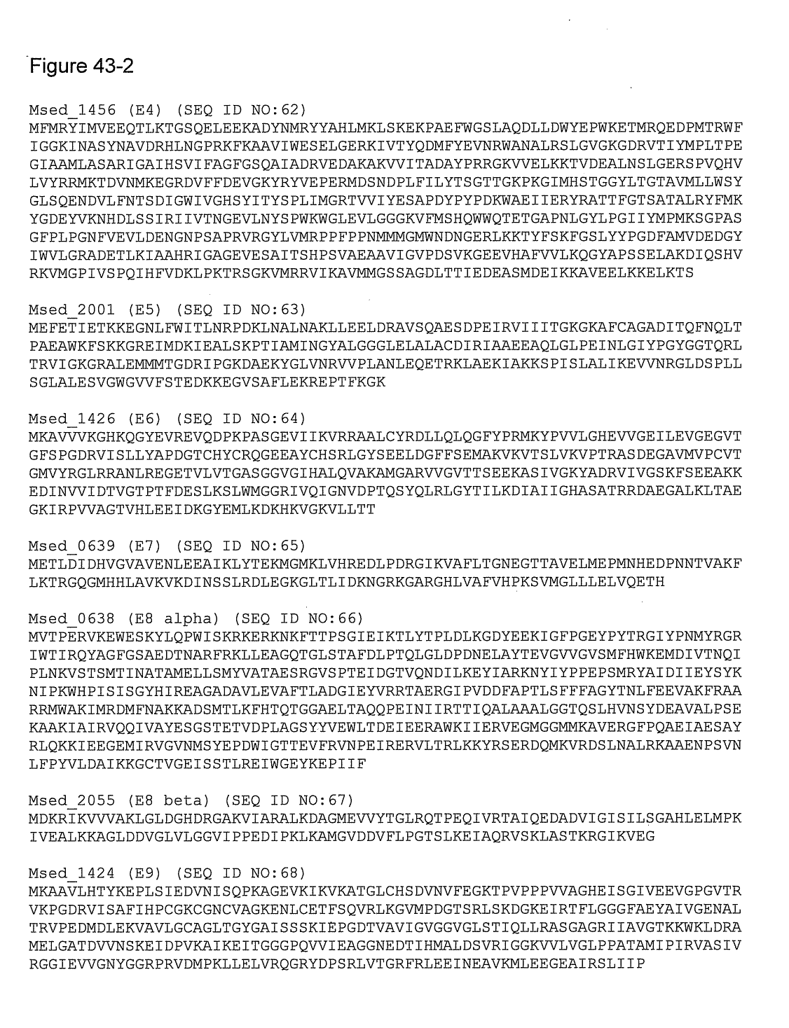

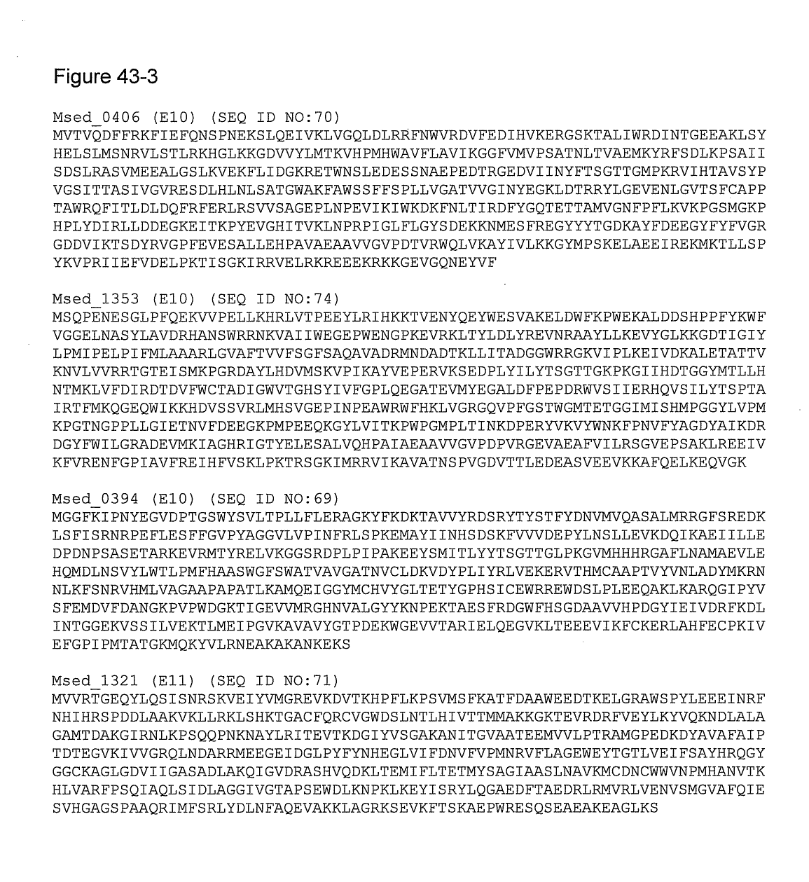

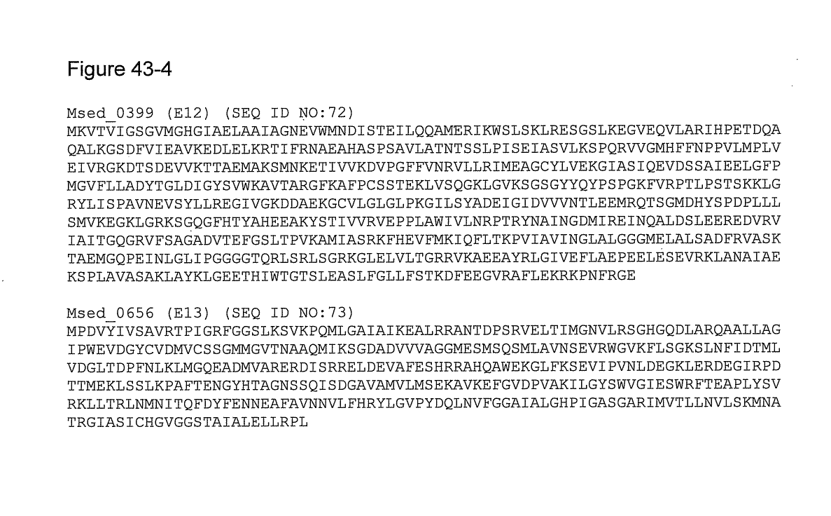

A bacterial promoter and coding regions encoding an RNA polymerase may be selected from a member of the domain Bacteria. In one embodiment, the bacterium may be a thermophile having a Tₒₚₜ of between 66° C. and 75° C. Examples of such bacteria include, but are not limited to, Caldicellulosiruptor saccharolyticus (Tₒₚₜ 70° C.), and Persephonella marina (Topt 73° C.). Other bacterial thermophiles having a Tₒₚₜ between 66° C. and 75° C. are readily available and may also be used as a source of bacterial promoters and RNA polymerases useful in the methods described herein. In one embodiment, the bacterium may be a thermophile having a Tₒₚₜ between 50° C. and 65° C. Examples of such bacteria include, but are not limited to, Clostridium thermocellum (Tₒₚₜ 60° C.), such as C. thermocellum JW20, which is available through the ATCC, and Petrotoga mobilis (Tₒₚₜ 55° C.), such as P. mobilis SJ95. Other bacterial thermophiles having a Tₒₚₜ between 50° C. and 65° C. are readily available and may also be used as a source of bacterial promoters and RNA polymerases useful in the methods described herein. Examples of suitable bacterial promoter/RNA polymerase combinations are shown in FIG. 42.

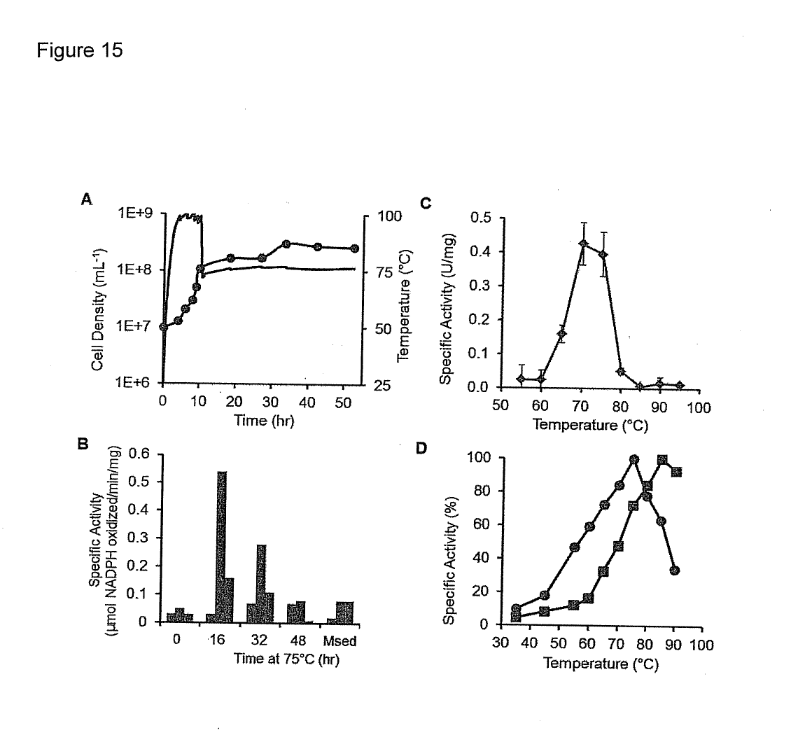

The polypeptide encoded by the coding region present on a heterologous polynucleotide is not intended to be limiting in any way. In one embodiment, the polypeptide is a heterologous polypeptide. In one embodiment, the coding region present on a heterologous polynucleotide encodes a polypeptide having greater activity at lower temperatures and lower activity at higher temperatures. In one embodiment, such a polypeptide has an optimal activity at a temperature of no greater than 75° C., no greater than 70° C., no greater than 65° C., no greater than 60° C., no greater than 55° C., no greater than 50° C., no greater than 45° C., no greater than 40° C., or no greater than 35° C. In one embodiment, such a polypeptide has an optimal activity at a temperature of at least 35° C., at least 40° C., at least 45° C., at least 50° C., at least 55° C., at least 60° C., at least 65° C., at least 70° C., or at least 75° C. In one embodiment, such a polypeptide has an optimal activity at a temperature between 35° C. and 45° C., between 40° C. and 50° C., between 45° C. and 55° C., between 50° C. and 60° C., between 55° C. and 65° C., between 60° C. and 70° C., or between 65° C. and 75° C. The optimal activity of many polypeptides is known, or can be readily determined by the skilled person using routine methods. In one embodiment, the optimal activity of a polypeptide is determined by expressing the polypeptide in an archaeon during growth at selected temperatures and then measuring activity of the polypeptide in an extract of the cell, for instance, as described in Example 3 (see FIG. 15C). For instance, when determining the activity of an enzyme produced by an archaeon at a selected temperature, the archaeon may be cultured at that temperature, such as between 55° C. and 90° C., for between 15 and 20 hours before the activity of the enzyme is measured at, for instance, 70° C.

In one embodiment, the coding region present on a heterologous polynucleotide encodes a polypeptide having optimal activity at a temperature that is below the Tₒₚₜ of the archaeon. In one embodiment, the optimal activity of such a polypeptide is no greater than 40° C., no greater than 30° C., no greater than 20° C., or no greater than 10° C. below the Tₒₚₜ of an archaeon. In one embodiment, the optimal activity of such a polypeptide is at least 10° C., at least 20° C., at least 30° C., or at least 40° C. below the Tₒₚₜ of an archaeon. In one embodiment, the optimal activity is between 10° C. and 20° C., between 15° C. and 25° C., between 20° C. and 30° C., between 25° C. and 35° C., or between 30° C. and 40° C. below the Tₒₚₜ of an archaeon. The tₒₚₜ of various archaea is known, or can be readily determined by the skilled person using routine methods. The Tₒₚₜ of Thermococcus kodakarensis is 85° C., the Tₒₚₜ of T. onnurineus is 85° C., the Tₒₚₜ of Sulfolobus solfataricus is 75° C., the Tₒₚₜ of S. islandicus is 75° C., the Tₒₚₜ of S. acidocaldarius is 78° C., and the Tₒₚₜ of Pyrococcus furiosus is 100° C.

In one embodiment, the coding region present on a heterologous polynucleotide encodes a polypeptide that catalyzes a reaction that results in a product. An example of such an embodiment is described in Example 1.

In one embodiment, the coding region present on a heterologous polynucleotide encodes a polypeptide that catalyzes a step in a metabolic pathway. The metabolic pathway may be catabolic or anabolic. The metabolic pathway may be a pathway that is normally present in an archaeon cell, or it may be a pathway that is not normally present in an archaeon cell. For instance, a polypeptide that catalyzes a reaction that results in a product is described in Example 1. In another example, the 4-hydroxybutyrate pathway described in Examples 2 and 3 is not normally present in the host P. furiosus cell. Examples of metabolic pathways include, but are not limited to, those involved in anaerobic respiration, fermentation, carbohydrate metabolism (including carbon fixation), lipid metabolism (such as fatty acid degradation, fatty acid synthesis, steroid metabolism, sphingolipid metabolism, eicosanoid metabolism, ketosis), and amino acid metabolism (including amino acids synthesis and amino acid degradation).

Examples of distinct pathways include the 4-hydroxybutyrate pathway (Berg et al., 2007, Science, 318:1782-1786; Examples 2 and 3), the acetone-butanol-ethanol pathway (Atsumi et al., 2008, Metab. Eng., 10:305-311; Chen and Hiu, 1986, Biotech. Lett., 8:371-376), the fatty acid ester pathway (Steen et al., 2010, Nature, 463:559-562), the pentose phosphate pathway, the glycolytic pathway, and the tricarboxylic acid cycle. The identity of individual enzymes of many pathways are known, including the amino acid sequence of each enzyme, and are readily available on the world wide web through databases including: the Reactome database of reactions, pathways, and biological processes; the MetaCyc database of metabolic pathways; the PathCase pathways database system; and the Database for Annotation, Visualization and Integrated Discovery Bioinformatics Resources.

The coding regions encoding polypeptides that make up a pathway, and are expressed in a genetically engineered archaeon (e.g., a polypeptide encoded by a coding region that is operably linked to a promoter and present on a heterologous polynucleotide) may be chosen from a microbe. The microbe used as a source of a polypeptide is not intended to be limiting. In one embodiment, polypeptides that make up a pathway are chosen from microbes having a Tₒₚₜ that is between 35° C. and 75° C. Polypeptides from such microbes are likely to have an optimal activity at a temperature that is between 35° C. and 75° C. The microbe used as a source of a polypeptide may be a member of the domain Archaea or the domain Bacteria. The microbe used as a source of a polypeptide may be mesophilic or thermophilic. A polypeptides that is part of a pathway may be produced using recombinant techniques.

A polynucleotide, such as a heterologous polynucleotide, disclosed herein may be present in a vector. A vector is a replicating polynucleotide, such as a plasmid, phage, or cosmid, to which another polynucleotide may be attached so as to bring about the replication of the attached polynucleotide. Construction of vectors containing a heterologous polynucleotide may employ standard ligation techniques known in the art. See, e.g., (Sambrook et al., 1989. Molecular cloning: a laboratory manual, 2nd ed. Cold Spring Harbor Laboratory Press, Cold Spring Harbor, N.Y.). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, and artificial chromosome vectors. Preferably the vector is a plasmid.

Selection of a vector depends upon a variety of desired characteristics in the resulting construct, such as a selection marker, vector replication rate, and the like. Vectors can be introduced into a host cell using methods that are known and used routinely by the skilled person for introduction of DNA into an archaeon. The vector may replicate separately from the chromosome present in the archaeon, or the polynucleotide may be integrated into a chromosome of the archaeon. When more than one vector is to be used in a cell, vectors having compatible origins of replication may be used (Adams et al. (US Patent Application 20110020875).

A vector introduced into a host cell to result in a genetically engineered archaeon optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence include, but are not limited to, sequences that confer resistance to kanamycin, ampicillin, chloramphenicol, tetracycline, streptomycin, and neomycin. Examples of nutritional markers useful with certain host cells, including hyperthermophilic archaea and thermophilic archaea, are disclosed in Lipscomb et al. (US Published Patent Application 20120135411). Examples include, but are not limited to, a requirement for uracil, histidine, or agmatine.

Polynucleotides described herein may be obtained from microbes, or produced in vitro or in vivo. For instance, methods for in vitro synthesis include, but are not limited to, chemical synthesis with a conventional DNAJRNA synthesizer. Commercial suppliers of synthetic polynucleotides and reagents for such synthesis are well known.

Provided herein are methods for using a genetically engineered archaeon. An advantage of certain embodiments results from the expression of one or more desirable polypeptides in an archaeon at a temperature that is below the archaeon's Tₒₚₜ. An archaeon incubated at a temperature below its optimal growth temperature shows less growth and has low metabolic activity. For instance, some metabolic processes, such as replication, decrease significantly at temperatures that are at least 20° C., at least 25° C., at least 30° C., at least 35° C., or at least 40° C. below an archaeon's Tₒₚₜ. However, if such an archaeon also includes polypeptides with optimal activity at a temperature at or near the lower incubation temperature, the archaeon is able to devote more cellular energy to the production of product at the lower temperature. In some embodiments, if the product or an intermediate produced in the metabolic pathway is normally utilized by the archaeon during growth at or near its Tₒₚₜ, the use of a lower temperature decreases the archaeon's ability to drain the amount of product or intermediate produced in the cell during incubation at the lower temperature.

In one embodiment, a method includes culturing a genetically engineered archaeon at a first temperature that is at or near its Tₒₚₜ. Examples of suitable first temperatures include, but are not limited to, 100° C., 98° C., 95° C., 90° C., 85° C., 80° C., or 75° C. The first temperature may be within 10° C. of its Tₒₚₜ, within 5° C. of its Tₒₚₜ, or at its Tₒₚₜ. For instance, if the Tₒₚₜ of the archaeon is 100° C., the first temperature may be between 90° C. and 110° C., between 95° C. and 105° C., or at 100° C. Likewise, if the Tₒₚₜ of the archaeon is 78° C., the first temperature may be between 68° C. and 88° C., between 73° C. and 83° C., or at 78° C. The incubation may continue for any time period, and in one embodiment the incubation may continue until the culture is in log phase (also referred to as exponential phase) or in stationary phase.

In one embodiment, a method may include shifting the culture to a second temperature. In one embodiment, the shift in temperature results in more of a polypeptide encoded by a heterologous polynucleotide. Without intending to be limited by theory, in an embodiment where a coding region on a heterologous polynucleotide is operably linked to a constitutive promoter, the shift in temperature has little effect on expression of the polypeptide; however, at the second temperature the polypeptide will be more stable and more active. Also, without intending to be limited by theory, in an embodiment where a coding region on a heterologous polynucleotide is operably linked to a cold shock promoter, the shift in temperature results in increased expression of the coding region and greater amounts of active polypeptide in the genetically engineered archaeon. In one embodiment, the shift in temperature results in less of a polypeptide encoded by a polynucleotide, such as an endogenous polypeptide. Without intending to be limited by theory, in an embodiment where a coding region, such as an endogenous coding region, is operably linked to a cold repressed promoter, the shift in temperature results in decreased expression of the coding region and less of the polypeptide encoded by the coding region in the genetically engineered archaeon.

The shift in temperature may be accomplished by any method, including transferring the culture to the second temperature and allowing it to slowly cool to the second temperature, or actively cooling to decrease the temperature more quickly. In one embodiment, the second temperature may be at least 10° C., at least 20° C., at least 30° C., or at least 40° C. below the Tₒₚₜ of the genetically engineered archaeon. In one embodiment, the second temperature is between 10° C. and 20° C., between 15° C. and 25° C., between 20° C. and 30° C., between 25° C. and 35° C., or between 30° C. and 40° C. below the Tₒₚₜ of the genetically engineered archaeon. In one embodiment, the culturing may occur at a temperature of no greater than 75° C., no greater than 70° C., no greater than 65° C., no greater than 60° C., no greater than 55° C., no greater than 50° C., no greater than 45° C., no greater than 40° C., or no greater than 35° C. below the Tₒₚₜ of the genetically engineered archaeon.

The value for the second temperature may be based on the temperature at which a polypeptide encoded by the heterologous polynucleotide has optimal activity. For instance, in an embodiment where a genetically engineered archaeon has a Tₒₚₜ of 100° C. and includes one heterologous polypeptide having an optimum activity at 72° C., the second incubation temperature may be at least 25° C. below the Tₒₚₜ of the genetically engineered archaeon, or may be between 20° C. and 30° C. or between 25° C. and 35° C. below the Tₒₚₜ of the genetically engineered archaeon; however, other temperatures may be used. When a genetically engineered archaeon includes more than one heterologous polypeptide, a second temperature may be selected that allows all the heterologous polypeptides to be active. For instance, in an embodiment where a genetically engineered archaeon has a Tₒₚₜ of 100° C. and includes heterologous polypeptides having optimum activities at different temperatures, for instance, 78° C. and 72° C., the second incubation temperature may be at least 20° C. below the Tₒₚₜ of the genetically engineered archaeon (e.g., the second temperature is no greater than 80° C.), or may be between 20° C. and 30° C. below the Tₒₚₜ of the genetically engineered archaeon (e.g., the second temperature is 80° C. to 70° C.); however, other temperatures may be used. In one embodiment, the temperature used is one that results in activity of the one or more polypeptides encoded by one or more heterologous polynucleotides present in the genetically engineered archaeon. In one embodiment, the temperature used is one that results in activity of each of the polypeptides encoded by one or more heterologous polynucleotides present in the genetically engineered archaeon. The activity of each of the one or more polypeptides encoded by one or more heterologous polynucleotides does not need to be optimal, instead, a suitable temperature is chosen such that the activity level of the one or more polypeptides is high enough to achieve the desired result, such as the production of a desired product.

The second temperature is maintained for a sufficient period of time. In one embodiment, the second temperature is maintained for at least 5 hours, at least 10 hours, at least 15 hours, at least 20 hours, or at least 25 hours. In one embodiment, the second temperature is maintained for no greater than 10 hours, no greater than 15 hours, no greater than 20 hours, no greater than 25 hours, or no greater than 30 hours. In one embodiment, the second temperature is maintained at least until the activity of a polypeptide encoded by the heterologous polynucleotide in the genetically engineered archaeon is increased compared to the activity of the polypeptide in the genetically engineered archaeon during growth at the first temperature (e.g., the Tₒₚₜ). In one embodiment, the activity is increased at least 2-fold, at least 5-fold, at least 10-fold, at least 15-fold, at least 20-fold, at least 25-fold, or at least 30-fold compared to the activity of the polypeptide in the genetically engineered archaeon during growth at the first temperature. In one embodiment, the increase in activity is no greater than 30-fold, no greater than 25-fold, no greater than 20-fold, no greater than 15-fold, no greater than 10-fold, no greater than 5-fold, or no greater than 2-fold compared to the activity of the polypeptide in the genetically engineered archaeon during growth at the first temperature. The activity of a polypeptide encoded by the heterologous polynucleotide may be determined by an assay suitable for measuring the activity the polypeptide, and assays useful for measuring activity of a polypeptide varies depending upon the polypeptide. The reaction rate of a polypeptide is typically measured when the polypeptide is present in the protein extract of cultured cells after they are harvested, suspended in a buffer such as 100 mM Tris/HCl, pH 8.0, and broken by physical means such as sonication or chemical means such as osmotic shock.

In one embodiment, the second temperature is maintained at least until the expression of a coding region present on the heterologous polynucleotide in the genetically engineered archaeon is increased compared to expression of the coding region in the genetically engineered archaeon during growth at the first temperature (e.g., the Tₒₚₜ). In one embodiment, the expression is increased at least 2-fold, at least 5-fold, at least 10-fold, at least 15-fold, at least 20-fold, at least 25-fold, or at least 30-fold compared to the expression of the coding region in the genetically engineered archaeon during growth at the first temperature. In one embodiment, the increase in expression is no greater than 30-fold, no greater than 25-fold, no greater than 20-fold, no greater than 15-fold, no greater than 10-fold, no greater than 5-fold, or no greater than 2-fold compared to the expression of the coding region in the genetically engineered archaeon during growth at the first temperature. The expression of a coding region in a genetically engineered archaeon may be determined by any suitable assay, including, but not limited to, measuring the level of mRNA.

In one embodiment, the second temperature is maintained at least until the amount of a polypeptide encoded by the heterologous polynucleotide in the genetically engineered archaeon is increased compared to the amount of the polypeptide in the genetically engineered archaeon during growth at the first temperature (e.g., the Tₒₚₜ). In one embodiment, the amount is increased at least 2-fold, at least 5-fold, at least 10-fold, at least 15-fold, at least 20-fold, at least 25-fold, or at least 30-fold compared to the amount of the polypeptide in the genetically engineered archaeon during growth at the first temperature. In one embodiment, the increase in the amount is no greater than 30-fold, no greater than 25-fold, no greater than 20-fold, no greater than 15-fold, no greater than 10-fold, no greater than 5-fold, or no greater than 2-fold compared to the amount of the polypeptide in the genetically engineered archaeon during growth at the first temperature. The amount of a polypeptide encoded by the heterologous polynucleotide may be determined by an assay suitable for measuring the amount the polypeptide including, but not limited to, western immunoblot.

In one embodiment, the methods for using a genetically engineered archaeon include processing the cell to result in a cell-free extract. The cell-free extract may be used for the production of a desirable product. A cell-free extract of a culture of a genetically engineered archaeon may be produced before the culture is exposed to a first temperature. In such an embodiment, the cell-free extract is exposed to a suitable first temperature, then shifted to a suitable second temperature. A cell-free extract of a culture of a genetically engineered archaeon may be produced after the culture is exposed to a first temperature. In such an embodiment, the culture is grown in the first temperature, and then processed to result in a cell-free extract. The cell-free extract is then exposed to a suitable second temperature. During incubation of a cell-free extract at the first and/or second temperature, the extract may be supplemented with appropriate cellular components, such as suitable t-RNAs, ATP, and the like.

In one embodiment, a genetically engineered archaeon is used to produce a product, such as lactate. An example of one method for making lactate is described in Example 1. In Example 1 a coding region encoding a polypeptide having lactate dehydrogenase activity was expressed in a genetically engineered archaeon, P. furiosus. The lactate dehydrogenase was from a hyperthermophilic microbe Caldicellulosiruptor bescii having a Tₒₚₜ of 78° C., and the coding region was operably linked to a cold shock promoter. Transferring the genetically engineered archaeon from 98° C. to 72° C. resulted in increased expression of the coding region, and increased activity and amounts of the lactate dehydrogenase.

In one embodiment, a genetically engineered archaeon includes one or more heterologous polynucleotides having coding regions operably linked to the promoters described herein, where the coding regions encode polypeptides that are part of a system for producing C2, C3, and/or C4 compounds from CO₂ and H₂. In one embodiment, the system is a complete cycle. This cycle, also referred to herein as the 4-hydroxybutyrate cycle, can be broken down into three sub-pathways, as shown in equations 1-3,

Acetyl CoA+CO₂+ATP+2H₂→3-HP+ADP+Pi+CoA [1]

3-HP+CO₂+2ATP+3H₂→4-HB+ADP+AMP+Pi+PPi [2]

4-HB+ATP+NAD++2CoA→2Acetyl CoA+AMP+PPi+NADH [3]

where 3-HP is 3-hydroxypropionate, and 4-HB is 4-hydroxybutyrate. The reaction described in equation 1 is also referred to herein as the 3-HP subpathway or SP1, and the reaction described in equation 2 is also referred to herein as the 4-HB subpathway or SP2. Thus, the system described herein can be used to produce 3-HP, 4-HB, acetyl CoA, or a combination thereof. In some embodiments other compounds may be produced, as described in greater detail herein.

In one embodiment, which is described by equation 1, the system includes a polypeptide having acetyl/propionyl-CoA carboxylase activity, a polypeptide having malonyl/succinyl-CoA reductase activity, and a polypeptide having malonate semialdehyde activity. In one aspect of this embodiment, the system produces 3-HP. Aspects of the production of 3-HP, including useful carbon donors and electron donors, are discussed herein.

A polypeptide having acetyl/propionyl-CoA carboxylase activity means the polypeptide catalyzes the conversion of acetyl CoA to malonyl-CoA or the conversion of propionyl-CoA to (S)-methylmalonyl-CoA. The acetyl/propionyl-CoA carboxylase activity of a polypeptide may be determined by routine methods known in the art.

An example of a polypeptide having acetyl/propionyl-CoA carboxylase activity is a heterotrimeric polypeptide that includes one amino acid sequence encoded by coding sequence Msed_{—}0147 of Genbank accession NC_{—}009440 and disclosed at SEQ ID NO:57, one amino acid sequence encoded by coding sequence Msed_{—}0148 of Genbank accession NC_{—}009440 and disclosed at SEQ ID NO:58, and one amino acid sequence encoded by coding sequence Msed_{—}1375 of Genbank accession NC_{—}009440 and disclosed at SEQ ID NO:59.

Other examples of polypeptides having acetyl/propionyl-CoA carboxylase activity include a polypeptide having structural similarity to the amino acid sequence encoded by coding sequence Msed_{—}0147 of Genbank accession NC_{—}009440 and disclosed at SEQ ID NO:57, a polypeptide having structural similarity to the amino acid sequence encoded by coding sequence Msed_{—}0148 of Genbank accession NC_{—}009440 and disclosed at SEQ ID NO:58, and/or a polypeptide having structural similarity to the amino acid sequence encoded by coding sequence Msed_{—}1375 of Genbank accession NC_{—}009440 and disclosed at SEQ ID NO:59. A candidate polypeptide having structural similarity to one of the polypeptides SEQ ID NO:57, 58, or 59 has acetyl/propionyl-CoA carboxylase activity when expressed in a microbe with the other 2 reference polypeptides. For instance, when determining if a candidate polypeptide having some level of identity to SEQ ID NO:57 has acetyl/propionyl-CoA carboxylase activity, the candidate polypeptide is expressed in a microbe with reference polypeptides SEQ ID NO:58 and 59. When determining if a candidate polypeptide having some level of identity to SEQ ID NO:58 has acetyl/propionyl-CoA carboxylase activity; the candidate polypeptide is expressed in a microbe with reference polypeptides SEQ ID NO:57 and 59. When determining if a candidate polypeptide having some level of identity to SEQ ID NO:59 has acetyl/propionyl-CoA carboxylase activity, the candidate polypeptide is expressed in a microbe with reference polypeptides SEQ ID NO:57 and 58.

Additional examples of polypeptides expected to have acetyl/propionyl-CoA carboxylase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25) and Chloroflexales (such as Chloreexus sp. Y-400-fl, C. aurantiacus J-10-fl, and C. aggregans DSM 9485).

A polypeptide having malonyl/succinyl-CoA reductase activity means the polypeptide catalyzes the conversion of malonyl-CoA tomalonate semialdehyde or succinyl-CoA to succinate semialdehyde. The malonyllsuccinyl-CoA reductase activity of a polypeptide may be determined by routine methods known in the art. An example of such a polypeptide includes an amino acid sequence encoded by coding sequence Msed_{—}0709 of Genbank accession NC_{—}009440 and disclosed at SEQ ID NO:60.

Other examples of polypeptides having malonyl/succinyl-CoA reductase activity include a polypeptide having structural similarity to the amino acid sequence encoded by coding sequence Msed_{—}0709 of Genbank accession NC_{—}009440 and disclosed at SEQ ID NO:60.

Additional examples of polypeptides expected to have malonyl/succinyl-CoA reductase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25) and Desulfurococcales (such as Ignicoccus hospitalis KIN4/I) and Euryarchaeotes (Thermococcales) (such as Pyrococcus sp. NA2), and Chloroflexales (such as Chloroflexus sp. Y-400-fl, C. aurantiacus J-10-fl, and C. aggregans DSM 9485).

A polypeptide having malonate semialdehyde activity means the polypeptide catalyzes the conversion of malonate semialdehyde to 3-hydroxypropionate. The malonate semialdehyde activity of a polypeptide may be determined by routine methods known in the art. An example of such a polypeptide includes one amino acid sequence encoded by coding sequence Msed_{—}1993 of Genbank accession NC_{—}009440 and disclosed at SEQ ID NO:61.

Other examples of polypeptides having malonate semialdehyde activity include a polypeptide having structural similarity to the amino acid sequence encoded by coding sequence Msed_{—}1993 of Genbank accession NC_{—}009440 and disclosed at SEQ ID NO:61.

Additional examples of polypeptides expected to have malonate semialdehyde activity may be obtained from members of the order Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N15.51, S. islandicus L.S.2.15, S. islandicus L.D.8.5, S. islandicus M16.4, S. solfataricus P2, and S. islandicus M14.25).

In one embodiment, which is described by equation 2, the system includes a polypeptide having 3-hydroxypropionate:CoA ligase activity, a polypeptide having 3-hydroxypropionyl-CoA dehydratase activity, a polypeptide having acryloyl-CoA reductase activity, a polypeptide having methylmalonyl-CoA epimerase activity, a polypeptide having methylmalonyl-CoA mutase activity, and a polypeptide having succinate semialdehyde reductase activity. In one aspect of this embodiment, the system produces 4-HB. Aspects of the production of 4-HB, including useful carbon donors and electron donors, are discussed herein.

A polypeptide having 3-hydroxypropionate:CoA ligase activity means the polypeptide catalyzes the conversion of 3-hydroxypropionate to 3-hydroxypropionyl CoA. The 3-hydroxypropionate:CoA ligase activity of a polypeptide may be determined by routine methods known in the art. An example of such a polypeptide includes an amino acid sequence encoded by coding sequence Msed_{—}1456 of Genbank accession NC_{—}009440 and disclosed at SEQ ID NO:62.

Other examples of polypeptides having 3-hydroxypropionate:CoA ligase activity include a polypeptide having structural similarity to the amino acid sequence encoded by coding sequence Msed_{—}1456 of Genbank accession NC_{—}009440 and disclosed at SEQ ID NO:62.

Additional examples of polypeptides expected to have 3-hydroxypropionate:CoA ligase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), Thermoproteales (such as Vulcanisaeta moutnovskia 768-28 and V. distributa DSM 14429), Acidilobales (such as Acidilobus saccharovorans 345-15), and Euryarchaeotes (Thermococcales) (such as Thermococcus sibiricus MM 739, T. barophilus MP, Pyrococcus furiosus DSM 3638, Pyrococcus sp. NA2, P. horikoshii OT3, Thermococcus gammatolerans EJ3).

A polypeptide having 3-hydroxypropionyl-CoA dehydratase activity means the polypeptide catalyzes the conversion of 3-hydroxypropionyl-CoA to acryloyl-CoA. The 3-hydroxypropionyl-CoA dehydratase activity of a polypeptide may be determined by routine methods known in the art. An example of such a polypeptide includes an amino acid sequence encoded by coding sequence Msed_{—}2001 of Genbank accession NC_{—}009440 and disclosed at SEQ ID NO:63.

Other examples of polypeptides having 3-hydroxypropionyl-CoA dehydratase activity include a polypeptide having structural similarity to the amino acid sequence encoded by coding sequence Msed_{—}2001 of Genbank accession NC_{—}009440 and disclosed at SEQ ID NO:63.

Additional examples of polypeptides expected to have 3-hydroxypropionyl-CoA dehydratase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), Thermoproteales (such as Vulcanisaeta distributa DSM 14429), Acidilobales (such as Acidilobus saccharovorans 345-15), and Desulfurococcales (such as Aeropyrum pernix K1).

A polypeptide having acryloyl-CoA reductase activity means the polypeptide catalyzes the conversion of acryloyl-CoA to propionyl-CoA. The acryloyl-CoA reductase activity of a polypeptide may be determined by routine methods known in the art. An example of such a polypeptide includes an amino acid sequence encoded by coding sequence Msed_{—}1426 of Genbank accession NC_{—}009440 and disclosed at SEQ ID NO:64.

Other examples of polypeptides having acryloyl-CoA reductase activity include a polypeptide having structural similarity to the amino acid sequence encoded by coding sequence Msed_{—}1426 of Genbank accession NC_{—}009440 and disclosed at SEQ ID NO:64.

Additional examples of polypeptides expected to have acryloyl-CoA reductase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), and Thermoproteales (such as Vulcanisaeta moutnovskia 768-28 and V. distributa DSM 14429).

A polypeptide having methylmalonyl-CoA epimerase activity means the polypeptide catalyzes the conversion of (S)-methylmalonyl-CoA to (R)-methylmalonyl-CoA. The methylmalonyl-CoA epimerase activity of a polypeptide may be determined by routine methods known in the art. An example of such a polypeptide includes an amino acid sequence encoded by coding sequence Msed_{—}0639 of Genbank accession NC_{—}009440 and disclosed at SEQ ID NO:65.

Other examples of polypeptides having methylmalonyl-CoA epimerase activity include a polypeptide having structural similarity to the amino acid sequence encoded by coding sequence Msed_{—}0639 of Genbank accession NC_{—}009440 and disclosed at SEQ ID NO:65.

Additional examples of polypeptides expected to have methylmalonyl-CoA epimerase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), Thermoproteales (such as Vulcanisaeta distributa DSM 14429), Euryarchaeotes (Thermococcales) (such as Thermococcus sibiricus MM 739, T. barophilus MP, Pyrococcus furiosus DSM 3638, Pyrococcus sp. NA2, P. horikoshii OT3, T. gammatolerans EJ3, P. abyssi GE5, and Thermococcus onnurineus NA1), and Chloroflexales (such as Chloroflexus sp. Y-400-fl, C. aurantiacus J-10-f1, and C. aggregans DSM 9485).

An example of a polypeptide having methylmalonyl-CoA mutase activity is a heterodimeric polypeptide that includes one amino acid sequence encoded by coding sequence Msed_{—}0638 of Genbank accession NC_{—}009440 and disclosed at SEQ ID NO:66, and one amino acid sequence encoded by coding sequence Msed_{—}2055 of Genbank accession NC_{—}009440 and disclosed at SEQ ID NO:67.

Other examples of polypeptides having methylmalonyl-CoA mutase activity include a polypeptide having structural similarity to the amino acid sequence encoded by coding sequence Msed_{—}0638 of Genbank accession NC_{—}009440 and disclosed at SEQ ID NO:66, and/or a polypeptide having structural similarity to the amino acid sequence encoded by coding sequence Msed_{—}2055 of Genbank accession NC_{—}009440 and disclosed at SEQ ID NO:67. A candidate polypeptide having structural similarity to one of the polypeptides SEQ ID NO:66 or 67 has methylmalonyl-CoA mutase activity when expressed in a microbe with the other reference polypeptide. For instance, when determining if a candidate polypeptide having some level of identity to SEQ ID NO:66 has methylmalonyl-CoA mutase activity, the candidate polypeptide is expressed in a microbe with reference polypeptides SEQ ID NO:67. When determining if a candidate polypeptide having some level of identity to SEQ ID NO:67 has methylmalonyl-CoA mutase activity, the candidate polypeptide is expressed in a microbe with reference polypeptides SEQ ID NO:66.

Additional examples of polypeptides expected to have methylmalonyl-CoA mutase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), Thermoproteales (such as Vulcanisaeta moutnovskia 768-28 and V. distributa DSM 14429), Acidilobales (such as Acidilobus saccharovorans 345-15), Desulfurococcales (such as Aeropyrum pernix K1), Euryarchaeotes (Thermococcales) (such as Thermococcus sibiricus MM 739, T. barophilus MP, Pyrococcus furiosus DSM 3638, Pyrococcus sp. NA2, P. horikoshii OT3, T. gammatolerans EJ3, P. abyssi GE5, and Thermococcus onnurineus NA1), and Chloroflexales (such as Chloroflexus sp. Y-400-fl, C. aurantiacus J-10-f1, and C. aggregans DSM 9485).

A polypeptide having succinate semialdehyde reductase activity means the polypeptide catalyzes the conversion of succinate semialdehyde to 4-hydroxybutyrate. The succinate semialdehyde reductase activity of a polypeptide may be determined by routine methods known in the art. An example of such a polypeptide includes an amino acid sequence encoded by coding sequence Msed_{—}1424 of Genbank accession NC_{—}009440 and disclosed at SEQ ID NO:68.

Other examples of polypeptides having succinate semialdehyde reductase activity include a polypeptide having structural similarity to the amino acid sequence encoded by coding sequence Msed_{—}1424 of Genbank accession NC_{—}009440 and disclosed at SEQ ID NO:68.

Additional examples of polypeptides expected to have semialdehyde reductase activity may be obtained from members of the order Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25).

In one embodiment, which is described by equation 3, the system includes a polypeptide having a polypeptide having 4-hydroxybutyrate:CoA ligase activity, a polypeptide having 4-hydroxybutyrl-CoA dehydratase activity, a polypeptide having crotonyl-CoA hydratase/(S)-3-hydroxybutyrl-CoA dehydrogenase activity, and a polypeptide having acetoacetyl-CoA (3-ketothiolase activity. In one aspect of this embodiment, the system produces acetyl-CoA. Aspects of the production of acetyl-CoA, including useful carbon donors and electron donors, are discussed herein.

A polypeptide having 4-hydroxybutyrate:CoA ligase activity means the polypeptide catalyzes the conversion of 4-hydroxybutyrate to 4-hydroxybutyryl-CoA. The 4-hydroxybutyrate:CoA ligase activity of a polypeptide may be determined by routine methods known in the art. An example of such a polypeptide includes an amino acid sequence encoded by coding sequence Msed_{—}0394 of Genbank accession NC_{—}009440 and disclosed at SEQ ID NO:69. Another example of a polypeptide having 4-hydroxybutyrate:CoA ligase activity includes an amino acid sequence encoded by coding sequence Msed_{—}0406 of Genbank accession NC_{—}009440 and disclosed at SEQ ID NO:70.

Other examples of polypeptides having 4-hydroxybutyrate:CoA ligase activity include a polypeptide having structural similarity to the amino acid sequence encoded by coding sequence Msed_{—}0394 of Genbank accession NC_{—}009440 and disclosed at SEQ ID NO:69 and a polypeptide having structural similarity to the amino acid sequence encoded by coding sequence Msed_{—}0406 of Genbank accession NC 009440 and disclosed at SEQ ID NO:70.

In one embodiment, an example of a polypeptide having 4-hydroxybutyrate:CoA ligase activity is an amino acid sequence encoded by coding sequence Msed_{—}1353 of Genbank accession NC_{—}009440 and disclosed at SEQ ID NO:74, provided that the amino acid at residue 424 is not the tryptophan present in a wild type Msed_{—}1353. In one embodiment, the amino acid at residue 424 is glycine. The amino acid sequence disclosed at SEQ ID NO:74 includes the substitution of glycine for tryptophan. Another example is a polypeptide having structural similarity to the amino acid sequence SEQ ID NO:74, provided the amino acid at residue 424 is not tryptophan.

Additional examples of polypeptides expected to have 4-hydroxybutyrate:CoA ligase activity include polypeptides catalyzing a CoA-ligase reaction that uses short (C2-C4) or medium (C5-C8) linear organic acids as a substrate. For instance, examples of polypeptides expected to have 4-hydroxybutyrate:CoA ligase activity include polypeptides catalyzing the reaction described under the IUBMB Enzyme Nomenclature system as EC 6.2.1.1, EC 6.2.1.3, EC 6.2.1.17, or EC 6.2.1.36. Such polypeptides may be obtained from members of the orders Desulfurococcales (such as Ignicoccus hospitalis, or Pyrolobus fumarii), Thermoproteales (such as Thermoproteus neutrophilus), or Sulfolobales (such as Sulfolobus acidocaldarius, S. islandicus, S. solfataricus, S. tokodaii, Metallosphaera cuprina, or M. sedula).

A polypeptide having 4-hydroxybutyryl-CoA dehydratase activity means the polypeptide catalyzes the conversion of 4-hydroxybutyryl-CoA to crotonyl-CoA. The 4-hydroxybutyryl-CoA dehydratase activity of a polypeptide may be determined by routine methods known in the art. An example of such a polypeptide includes an amino acid sequence encoded by coding sequence Msed_{—}1321 of Genbank accession NC_{—}009440 and disclosed at SEQ ID NO:71.

Other examples of polypeptides having 4-hydroxybutyryl-CoA dehydratase activity include a polypeptide having structural similarity to the amino acid sequence encoded by coding sequence Msed_{—}1321 of Genbank accession NC_{—}009440 and disclosed at SEQ ID NO:71.

Additional examples of polypeptides expected to have 4-hydroxybutyryl-CoA dehydratase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), and Desulfurococcales (such as Ignicoccus hospitalis KIN4/I).

A polypeptide having crotonyl-CoA hydratase/(S)-3-hydroxybutyryl-CoA dehydrogenase activity means the polypeptide catalyzes the conversion of crotonyl-CoA to acetoacetyl-CoA. The crotonyl-CoA hydratase/(S)-3-hydroxybutyrl-CoA dehydrogenase activity of a polypeptide may be determined by routine methods known in the art. An example of such a polypeptide includes an amino acid sequence encoded by coding sequence Msed_{—}0399 of Genbank accession NC_{—}009440 and disclosed at SEQ ID NO:72.

Other examples of polypeptides having crotonyl-CoA hydratase/(S)-3-hydroxybutyrl-CoA dehydrogenase activity include a polypeptide having structural similarity to the amino acid sequence encoded by coding sequence Msed_{—}0399 of Genbank accession NC_{—}009440 and disclosed at SEQ ID NO:72.

Additional examples of polypeptides expected to have crotonyl-CoA hydratase/(S)-3-hydroxybutyrl-CoA dehydrogenase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), Thermoproteales (such as Vulcanisaeta moutnovskia 768-28 and V. distributa DSM 14429), Acidilobales (such as Acidilobus saccharovorans 345-15), and Desulfurococcales (such as Aeropyrum pernix K1, and Ignicoccus hospitalis KIN4/I).

A polypeptide having acetoacetyl-CoA β-ketothiolase activity means the polypeptide catalyzes the conversion of acetoacetyl-CoA to Acetyl-CoA. The acetoacetyl-CoA β-ketothiolase activity of a polypeptide may be determined by routine methods known in the art. An example of such a polypeptide includes an amino acid sequence encoded by coding sequence Msed_{—}0656 of Genbank accession NC_{—}009440 and disclosed at SEQ ID NO:73.

Other examples of polypeptides having acetoacetyl-CoA β-ketothiolase activity include a polypeptide having structural similarity to the amino acid sequence encoded by coding sequence Msed_{—}0656 of Genbank accession NC_{—}009440 and disclosed at SEQ ID NO:74.

Additional examples of polypeptides expected to have acetoacetyl-CoA β-ketothiolase dehydrogenase activity may be obtained from members of the orders Sulfolobaceae (such as Metallosphaera sedula DSM 5348 and M. cuprina Ar-4, Acidianus hospitalis W1, Sulfolobus tokodaii str. 7, S. acidocaldarius DSM 639, S. islandicus Y.G.57.14, S. islandicus Y.N.15.51, S. islandicus L.S.2.15, S. islandicus L.D.8.5, S. islandicus M16.4, S. solfataricus P2, and S. islandicus M14.25), Thermoproteales (such as Vulcanisaeta moutnovskia 768-28 and V. distributa DSM 14429), Acidilobales (such as Acidilobus saccharovorans 345-15), and Desulfurococcales (such as Aeropyrum pernix K1, and Ignicoccus hospitalis KIN4/I).

A candidate polypeptide (e.g., a polypeptide having structural similarity to a polypeptide described herein) may be isolated from a microbe, such as a thermophile or a hyperthermophile. A candidate polypeptide may be produced using recombinant techniques, or chemically or enzymatically synthesized.

A polypeptide described herein may be expressed as a fusion polypeptide that includes a polypeptide described herein and a heterologous polypeptide, such as a short amino acid sequence. The heterologous polypeptide may be present at the amino terminal end or the carboxy terminal end of a polypeptide, or it may be present within the amino acid sequence of the polypeptide. For instance, the heterologous amino acid sequence may be useful for purification of the fusion polypeptide by affinity chromatography. Various methods are available for the addition of such affinity purification tags to proteins. Examples of tags include a polyhistidine-tag, maltose-binding protein, and Strep-tag®. Representative examples may be found in Hopp et al. (U.S. Pat. No. 4,703,004), Hopp et al. (U.S. Pat. No. 4,782,137), Sgarlato (U.S. Pat. No. 5,935,824), Sharma (U.S. Pat. No. 5,594,115), and Skerra and Schmidt, 1999, Biomol Eng. 16:79-86). The heterologous amino acid sequence, for instance, a tag or a carrier, may also include a cleavable site that permits removal of most or all of the additional amino acid sequence. Examples of cleavable sites are known to the skilled person and routinely used, and include, but are not limited to, a TEV protease recognition site. The number of heterologous amino acids may be, for instance, at least 5, at least 10, at least 15, at least 20, at least 25, at least 30, at least 35, or at least 40.

The polypeptides described herein may be produced by produced using recombinant, synthetic, or chemical techniques. For instance, a polypeptide may be synthesized in vitro, e.g., by solid phase peptide synthetic methods. Solid phase peptide synthetic methods are routine and known in the art. A polypeptide produced using recombinant techniques or by solid phase peptide synthetic methods may be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity. A preferred method for isolating and optionally purifiying a hydrogenase polypeptide described herein includes column chromatography using, for instance, ion exchange chromatography, such as DEAE sepharose, hydrophobic interaction chromatography, such as phenyl sepharose, or the combination thereof.

The skilled person will recognize that the coding regions encoding the polypeptides described herein are readily available. For instance, a polynucleotide encoding a polypeptide represented by one of the sequences disclosed herein, e.g., SEQ ID NOs:57-74, is available as a coding region of Genbank accession NC_{—}009440 (the complete genomic sequence of the Metallosphaera sedula chromosome). It should be understood that a polynucleotide encoding a polypeptide represented by one of the sequences disclosed herein, e.g., SEQ ID NOs:57-74, is not limited to the nucleotide sequence disclosed as a coding region of Genbank accession NC_{—}009440, but also includes the class of polynucleotides encoding such polypeptides as a result of the degeneracy of the genetic code. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

In some embodiments, including, but not limited to, those embodiments where a genetically engineered archaeon includes one or more sub-pathways of the 4HB cycle, a genetically engineered archaeon optionally includes a source of electrons that can be used for the reduction of CO₂ and/or other intermediates in a metabolic pathway, such as the 4-HB cycle. In one embodiment, a source of electrons is hydrogenase, which catalyzes the reversible interconversion of H₂, protons, and electrons. A genetically engineered archaeon may naturally include a hydrogenase suitable for supplying reductant, and in one embodiment, such a genetically engineered archaeon may express a heterologous hydrogenase polypeptide at an increased level or have altered activity. For instance, a genetically engineered archaeon may include a heterologous promoter operably linked to one or more coding regions encoding subunits of a hydrogenase polypeptide. In another example, a heterologous polynucleotide encoding a subunit of a hydrogenase polypeptide may include a mutation, such as a deletion, an insertion, a transition, a transversion, or a combination thereof, that alters a characteristic of the hydrogenase polypeptides, such as the activity.

In one embodiment, a genetically engineered archaeon may include heterologous polypeptides encoding the subunits of a hydrogenase. Examples of hydrogenases and their expression in microbes are described in Adams et al. (US Patent Application 20110020875), and Chandrayan et al. (2012, J. Biol. Chem., 287(5):3257-3264).

A genetically engineered archaeon may include heterologous polynucleotides having coding regions that encode one or more of the polypeptides involved in the 4-hydroxybutyrate pathway. In one embodiment, the genetically engineered archaeon produces polypeptides for subpathway 1, subpathway 2, subpathway 3, or a combination thereof. In one embodiment, a combination is subpathway 1 and subpathway 2. In one embodiment, a combination is subpathway 1, subpathway 2, and subpathway 3. In one embodiment, a combination is subpathway 2 and subpathway 3. In one embodiment, a combination is subpathway 1 and subpathway 3. The polypeptides are incubated under conditions suitable for producing desirable products such 3-HP, 4-HB, and/or other products.

A method for using a genetically engineered archaeon may also include recovery of the product produced by the genetically engineered archaeon. Examples of products that may be produced by a genetically engineered archaeon include, but are not limited to, alcohols, such as ethanol, butanol, a diol, and organic acids such as lactic acid, acetic acid, formic acid, citric acid, oxalic acid, and uric acid. In one embodiment, the methods disclosed herein may be used to make 3-HP, 4-HB, and other products. The 4-HB cycle results in the production of acetyl CoA. Acetyl CoA is the ideal product as it represents an activated reduced C-2 unit that is of fundamental importance in conventional biosynthetic pathways. For example, acetyl CoA is the building block for the biosynthesis of fatty acids, polyisoprenoids and hydroxyacids (such as 3-BB), all of which are potential sources of alkane-based fuels and/or plastics. Thus, the 4-HB cycle can be used to directly generate a range of biofuels, including alkanes, biodiesel (fatty acid esters) and ethanol, as well as butanol. Moreover, when converted to pyruvate by reductive carboxylation, acetyl CoA can serve as the primary carbon and electron source for all known biofuels (Connor et al., 2009, Curr. Opin Biotechnol 20:307-315, Lee et al., 2008, Curr Opin Biotechnol 19:556-63, Peralta-Yahya et al., Biotechnol J 5:147-62). Other products that may be produced include, but are not limited to, 1,4-butanediol, succinic acid, and isopropanol. The method used for recovery depends upon the product, and methods for recovering products resulting from microbial pathways, including fermentation, are known to the skilled person and used routinely. For instance, when the product is ethanol, the ethanol may be distilled using conventional methods. For example, after fermentation the product, e.g., ethanol, may be separated from the fermented slurry. The slurry may be distilled to extract the ethanol, or the ethanol may be extracted from the fermented slurry by micro or membrane filtration techniques.

The present invention is illustrated by the following examples. It is to be understood that the particular examples, materials, amounts, and procedures are to be interpreted broadly in accordance with the scope and spirit of the invention as set forth herein.

### Example 1

Microorganisms growing near the boiling point have enormous biotechnological potential but only recently have molecular engineering tools become available for them. Described here is the engineering of the hyperthermophilic archaeon Pyrococcus furiosus, which grows optimally at 100° C., to switch its end products of fermentation in a temperature-controlled fashion without the need for chemical inducers. The recombinant strain (LAC) expresses a gene (ldh) encoding lactate dehydrogenase from the hypertheimophilic Caldicellulosiruptor bescii (optimal growth temperature [Tₒₚₜ] of 78° C.) controlled by a “cold shock” promoter that is upregulated when cells are transferred from 98° C. to 72° C. At 98° C., the LAC strain fermented sugar to produce acetate and hydrogen as end products, and lactate was not detected. When the LAC strain was grown at 72° C., up to 3 mM lactate was produced instead. Expression of a coding region from a moderately thermophilic bacterium in a hyperthermophilic archaeon at temperatures at which the hyperthermophile has low metabolic activity provides a new perspective to engineering microorganisms for bioproduct and biofuel formation.

The availability of a genetic system for an organism growing optimally at 100° C. opens up the possibility of engineering the temperature-dependent heterologous expression of genes encoding enzymes that are active at suboptimal growth temperatures for the host. Depending on the temperature, the host organism can have lower metabolic activity or be virtually inactive. For example, the generation time of P. furiosus increases from less than 1 h at 98° C. to about 7 h at 72° C. (Weinberg et al., 2005. J. Bacteriol. 187:336-348), with little growth below 65° C. (Fiala and Stetter, 1986. Arch. Microbiol. 145:56-61). Production of enzymes optimally active near 70° C. or so could give P. furiosus new metabolic capabilities at this temperature that it does not have at the optimum near 100° C. where the heterologously produced enzymes would likely be inactive. Similarly, at even lower temperatures (≦60° C.), P. furiosus could be a nonmetabolizing host, and chemical conversions could be accomplished only by the heterologously produced enzymes. While heterologous gene expression has already been reported using the related hyperthermophile, T. kodakarensis (Matsumi et al., 2007. J. Bacteriol. 189:2683-2691, Takemasa et al., 2011. Appl. Environ. Microbiol. 77:2392-2398), this involved genes from archaeal species that grow at temperatures comparable to that of T. kodakarensis. In this study, our goal was to heterologously express in P. furiosus a gene from a bacterium that grows at significantly lower temperature and determine if a new end product could be produced at lower incubation temperatures in the absence of any chemical inducer for gene expression.

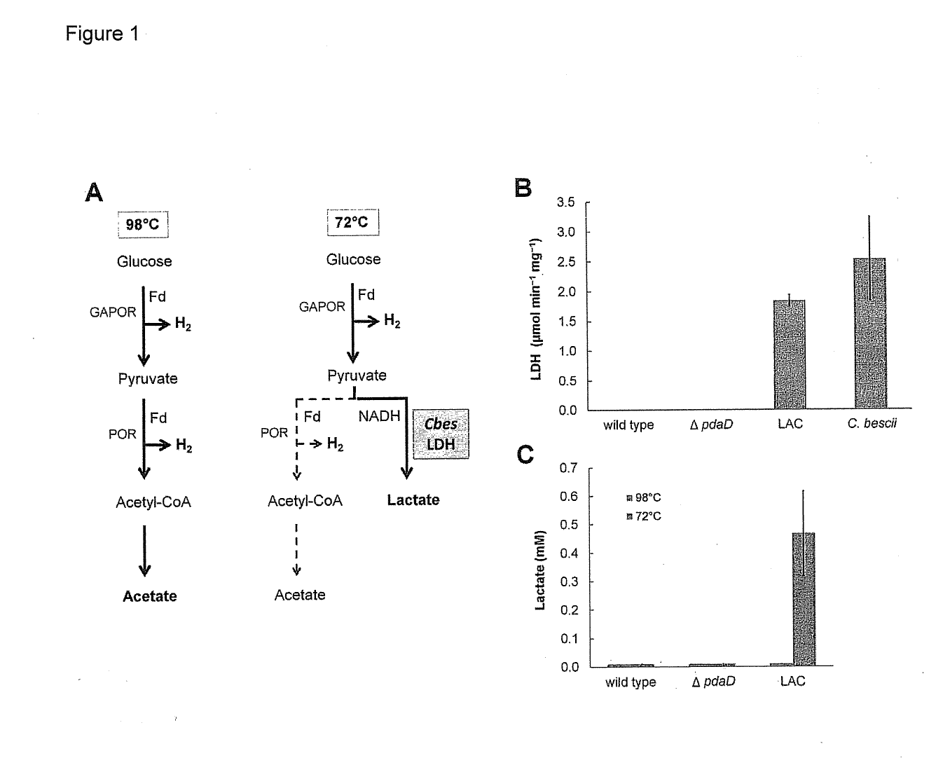

For proof of principle of a temperature-dependent metabolic switch in P. furiosus, we selected a bacterial gene that has no homolog in the P. furiosus genome and one that is involved in the metabolism of a compound that P. furiosus is not known to produce. The anaerobic bacterium Caldicellulosiruptor bescii grows optimally at 78° C. by sugar fermentation and produces lactate at millimolar concentrations as the main end product (Yang et al. 2009. Appl. Environ. Microbiol. 75:4762-4769). Lactate is generated by the reduction of pyruvate catalyzed by an NADH-dependent lactate dehydrogenase (LDH) encoded by Cbes_{—}1918 (ldh). In contrast, while P. furiosus also ferments sugars to pyruvate, its genome does not contain a gene encoding an LDH homolog, and the organism oxidizes pyruvate by pyruvate ferredoxin oxidoreductase to produce acetate, CO₂, and H₂ as the primary products (FIG. 1A). The goal was therefore to express the LDH gene of C. bescii in P. furiosus under control of the P_{cipA} promoter and determine whether any lactate is produced during growth at 72° C., but not at 98° C.

### Materials and Methods

Strains and Media.

Pyrococcus furiosus strains used in this study are listed in Table 1. In the transformation experiments, P. furiosus (DSM 3638) was cultured with 5 g liter⁻¹ maltose as the primary electron donor on liquid and solid complex medium as previously described (Lipscomb et al. 2011. Environ. Microbiol. 77:2232-2238). In all other experiments, the same medium was used, except that it contained no casein, but a yeast extract concentration of 2 g liter⁻¹ (Weinberg et al., 2005. J. Bacteriol. 187:336-348). For the cultivation of the ΔpdaD mutant strain, 4 mM agmatine (Sigma Chemical, St. Louis, Mo.) was added, while the medium for the COM1 and LAC strains was supplemented with 20 μM uracil (Table 1). Adapted growth at 72° C. and the temperature shock experiment were performed in a 20-liter custom fermenter as described previously (Weinberg et al., 2005. J. Bacteriol. 187:336-348). In the temperature shock experiment, the whole culture (15 liters) was rapidly cooled from 94° C. to 72° C. within 10 min. Caldicellulosiruptor bescii was grown on complex medium with 5 g liter⁻¹ cellobiose as an electron donor as described previously (Yang et al. 2010. Int. J. Syst. Evol. Microbiol., 60:2011-2015). Culture growth was in general followed by cell counting and by determination of protein concentration in subsamples.

TABLE 1
Pyrococcus furiosus strains used in Example 1

| | Relevant | Parent | Require- | |
|---|---|---|---|---|
| Strain | genotype | strain | ment | Source |
| DSM 3638 | Wild type | NA^{a} | NA | 9 |
| COM1 | ΔpyrF | DSM 3638 | Uracil | 8 |
| | | | (20 μM) | |
| ΔpdaD | ΔpyrF | COM1 | Agmatine | This |
| | ΔpdaD::P_{gdh}pyrF | | (4 mM) | study |
| LAC | ΔpyrF | ΔpdaD | Uracil | This |
| | ΔpdaD::pdaD | | (20 μM) | study |
| | P_{cipA}Cbes-ldh | | | |

^{a}Not available

Genetic Manipulations.

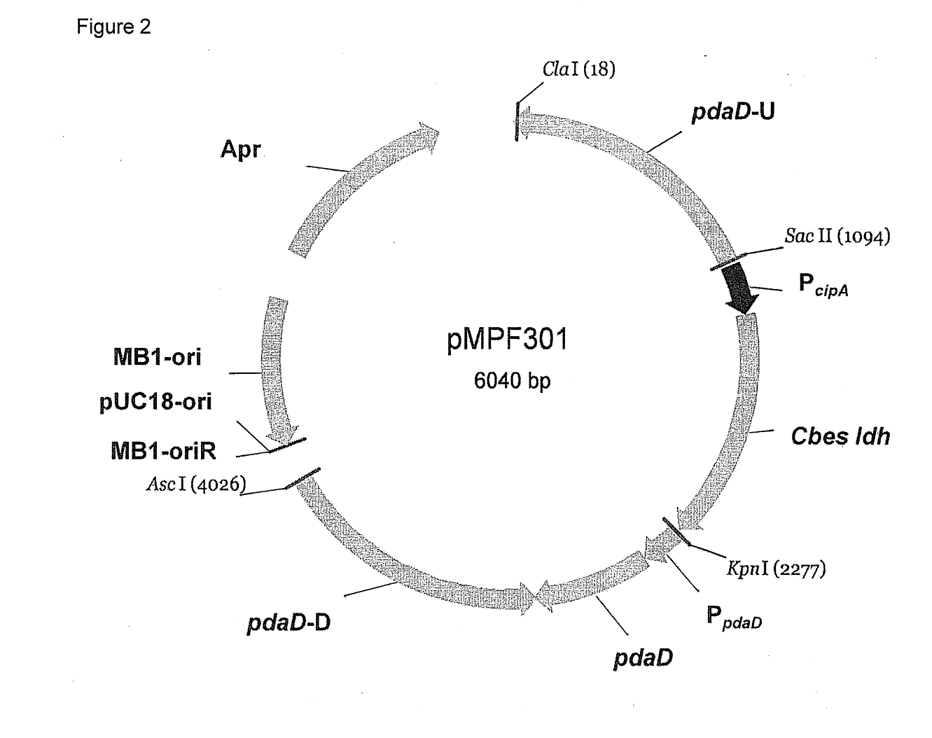

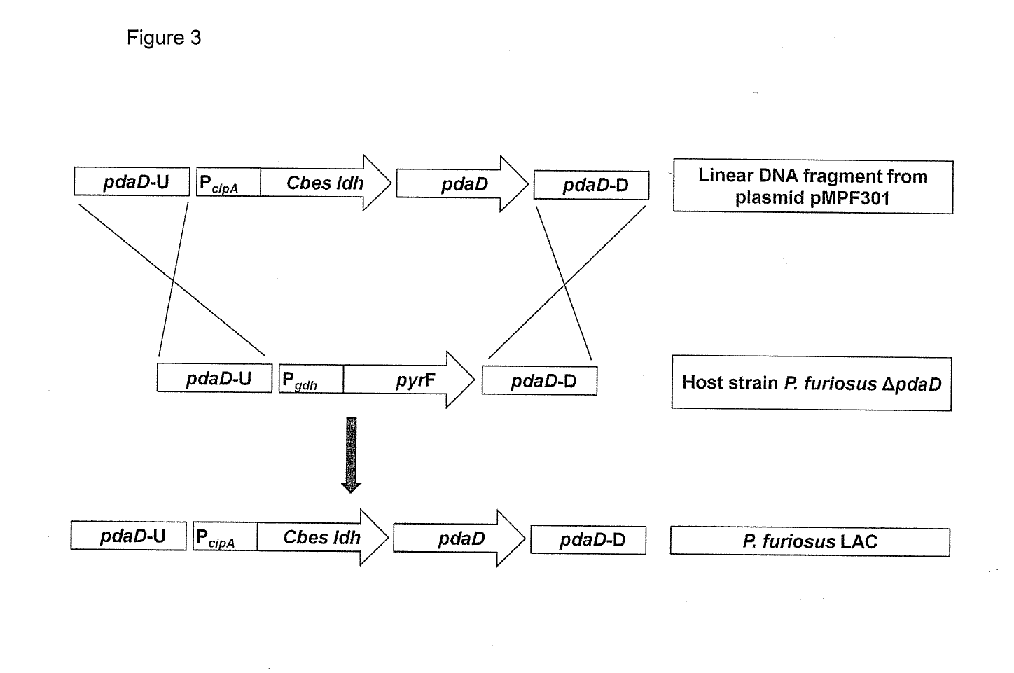

Extraction of DNA from C. bescii was performed by the method of Zhou et al. (Zhou et al., 1995. Int. J. Syst. Bacteriol. 45:500-506). Extraction of DNA from P. furiosus, transformation of P. furiosus, and selection of genetically modified strains were performed as previously described (Lipscomb et al. 2011. Appl. Environ. Microbiol. 77:2232-2238). P. furiosus COM1 served as the parent strain for genetic manipulations. A deletion of the pyruvoyl-dependent arginine decarboxylase (pdaD) gene (PF1623) was achieved by homologous recombination with the P_{gdh}pyrF cassette (Hopkins et al., 2011. PLoS One 6:e26569). The resulting strain, P. furiosus ΔpdaD strain, was used as the parent strain for the heterologous expression of the putative 1-lactate dehydrogenase of C. bescii (Cbes1918; Cbes-ldh). Cbes-ldh was amplified by PCR using the primer set Cbes1918-F (F stands for forward) and Cbes1918-KpnI-R (R stands for reverse). The cold-induced promoter P_{cipA} was amplified from genomic DNA from P. furiosus DSM3638 with the primer set P_{cipA}-SacII-F and P_{cipA}-Cbes1918-R. Finally, the fusion product P_{cipA}Cbes1918 was obtained by overlapping PCR using both products from the PCRs above and the primers P_{cipA}-SacII-F and Cbes1918-KpnI-R. The fusion product was introduced between the SacII site and the KpnI site of the plasmid vector pSPF300 (Hopkins et al., 2011. PLoS One 6:e26569), which additionally contained the pdaD gene and 1 kb upstream and downstream regions of pdaD. The resulting plasmid pMPF301 (FIG. 2) was amplified in Escherichia coli XL1 Blue-MRF′ (Stratagene, now Agilent Technologies, Santa Clara, Calif.) applying general genetic techniques (Sambrook J, Russell D W (ed). 2001. Molecular cloning: a laboratory manual, 3rd ed. Cold Spring Harbor Press, Cold Spring Harbor, N.Y.). The plasmid was digested with the ClaI and AscI restriction endonucleases, the larger fragment including the pdaD P_{cipA}Cbes-ldh cassette was purified with the Strataprep DNA gel extraction kit (Agilent Technologies) and subsequently used for transformation of the P. furiosus ΔpdaD strain (FIG. 3). Verification of the insertion of the pdaD P_{cipA}Cbes-ldh cassette into the chromosome was achieved by PCR with the primer set PF1623L-F and PF1623R-R located upstream and downstream of the cassette and subsequent sequencing. All primers used for PCR are listed in Table 2.

TABLE 2
Primers used in this study for PCR amplification and qPCR.

| Primer | Sequence (5′-3′) | Source |
|---|---|---|
| P_{cipA}-SacII-F | GAATCCCCGCGGTGACCTTTTATCCATTACTAACTTGC (SEQ ID NO: 31) | This study |
| P_{cipA}-Cbes1918-R | CAATAATTACAATTTTACCCGGTTTTCTCATTGCATATCACCTGCCAGGTATCTC | This study |
| | (SEQ ID NO: 32) | |
| Cbes1918-F | CCTGGCAGGTGATATGCAATGAGAAAACCGGGTAAAATTGTAATTATTGGAAC | This study |
| | (SEQ ID NO: 33) | |
| Cbes1918-KpnI-R | TCGGTTGGTACCAGCCTCCTATTATAGTTTTAAAGACTCTATCACAC | This study |
| | (SEQ ID NO: 34) | |
| PF1623L-F | GGAGCTCTGTTGCTTCTGCTAGAG (SEQ ID NO: 35) | This study |
| PF1623R-R | CTTTTCACCTACTATCTGCTCAAATGC (SEQ ID NO: 36) | This study |
| PF0971-qF | CGTTGTTGTTGTGCTAGATCC (SEQ ID NO: 37) | 1 |
| PF0971-qR | GATGGCTTCCTCTATGCTCTC (SEQ ID NO: 38) | 1 |
| Cbes1918-qF | GGGCGAACATGGAGACAGTGAAATTG (SEQ ID NO: 39) | This study |
| Cbes1918-qR | GCCAATGCAATGGCGTAATATGTTGC (SEQ ID NO: 40) | This study |

1: Lipscomb et al. 2011. Appl. Environ. Microbiol. 77:2232-2238

Preparation of Cell Extracts and Enzyme Assays.

P. furiosus and C. bescii cells were harvested by centrifugation for 10 min at 6,000 g. C. bescii cells were resuspended in 50 mM Tris (pH 8) and disrupted by sonication (five times, 2 min each time, maximum of 36 W and discontinuous operation at 50% of time). The P. furiosus cells were lysed by osmotic shock in 50 mM Tris HCl (pH 8.0) and 2 mM sodium dithionite. The lysis buffer contained 50 mg/ml DNase I (Sigma) to decrease the viscosity of the protein extract. Fractionation of the resulting protein extract into the soluble (cytoplasmic) fraction and the membrane fraction was achieved by ultracentrifugation at 100,000 g for 1 h. The membrane fraction was washed once with 50 mM Tris (pH 8.0) in order to minimize contamination with soluble proteins. Lactate dehydrogenase (LDH) (EC 1.1.1.27) activity was determined photometrically by the oxidation of NADH (340 nm) concomitant with lactate formation according to the following chemical equation: NADH+pyruvate+H⁺→NAD⁺+lactate. The assays were performed aerobically in closed glass cuvettes at 75° C., which contained 2.5 mM NADH in 50 mM sodium phosphate buffer (pH 7.0). The rate of nonspecific oxidation of NADH was determined before the reaction was started by the addition of 5 mM pyruvate. As internal controls for the quality of the P. furiosus protein extracts, glutamate dehydrogenase (GDH) (EC 1.4.1.2) activity was routinely measured by the formation of NADPH (340 nm) according to the following chemical equation: NADP⁺+glutamate+H₂O→2-oxoglutarate+NH₄⁺+NADPH. The GDH assay was the same as for LDH except that NADH was exchanged for NADP⁺ (0.25 mM), and pyruvate was exchanged for glutamate (5 mM). The protein content of the cell-free extracts were determined by the method of Bradford (Bradford 1976. Anal. Biochem. 72:248-254).

RNA Extraction and Quantitative PCR.

Cells were harvested for RNA extraction in the late logarithmic to early stationary phase of the growth curve unless noted otherwise. Cells were centrifuged for 10 min at 6,000 g and frozen until further processing. RNA was extracted using the Absolute RNA miniprep kit (Agilent Technologies), including a DNA digestion step with Turbo DNase (Ambion, Austin, Tex.) for 30 min at 37° C. cDNA was prepared using the Affinity Script cDNA synthesis kit (Agilent Technologies). All quantitative reverse transcription-PCRs (qRT-PCRs) were performed with an Mx3000P instrument (Stratagene), using the Brilliant Sybr green QPCR master mix (Agilent Technologies). The gamma subunit of the constitutively transcribed gene encoding the pyruvate-ferredoxin oxidoreductase (Schut et al., 2003. J. Bacteriol. 185:3935-3947) (PF0971) was used as an internal control to calculate the relative mRNA level of Cbes-ldh. Primers for qRT-PCR were designed using the Vector NTI software (Invitrogen). The amplicon sizes were 194 bp and 267 bp for Cbes-ldh and PF0971, respectively. Primers were tested for nonspecific products, and all experiments included controls without the addition of reverse transcriptase in the cDNA synthesis step to test for DNA contamination. The comparative cycle threshold method was used to analyze the resulting data, which are expressed as a ratio of gene expression change (n-fold). All primers used in qRT-PCR experiments are listed in Table 2.

Chemical Analyses.

L-Lactic acid was determined by using the Megazyme 1-lactic assay kit (Megazyme, Wicklow, Ireland). Acetate was determined by high-performance liquid chromatography (HPLC) on a model 2690 separations module (Waters, Milford, Mass.) equipped with an Aminex HPX-87H column (300 mm by 7.8 mm; Bio-Rad, Hercules, Calif.) and a photodiode array detector (model 996; Waters). The system was operated with 5 mM H₂SO₄ as the eluent at a flow rate of 0.6 ml min⁻¹. Samples for HPLC were acidified with 0.1 M H₂SO₄ and centrifuged before analysis to remove particles. Hydrogen was determined on a GC-8A gas chromatograph (Shimadzu, Kyoto, Japan) equipped with a thermal conductivity detector and a molecular sieve column (model 5A 80/100; Alltech, Deerfield, Ill.) with argon as the carrier gas.

### Results

To construct a P. furiosus strain containing the C. bescii ldh gene under control of the P_{cipA} promoter, the P_{cipA}Cbes-ldh gene fusion was cloned in the plasmid vector pSPF300 in Escherichia coli (FIG. 2 and Table 1). The agmatine-requiring P. furiosus mutant strain, ΔpdaD strain, was used as the host (Hopkins et al., 2011. PLoS One 6:e26569). This strain is deficient in agmatine biosynthesis, as the pdaA gene is replaced by pyrF, an essential gene for uracil biosynthesis (Table 1). The linearized plasmid containing P_{cipA}Cbes-ldh (FIG. 2) was used to complement the pdaD gene into the P. furiosus chromosome by replacing the pyrF gene by homologous recombination. The resulting transformants (ApyrF) required uracil but did not require agmatine for growth (FIG. 3). Plasmid integration was confirmed by PCR and for one colony by DNA sequencing. The recombinant P. furiosus strain was named LAC (Table 1). To investigate the expression of C. bescii ldh in P. furiosus and the production of lactate, the LAC strain was grown in batch culture under three different conditions: (i) in closed, static cultures (400-ml scale) at 72° C. and at 98° C. with no pH control; (ii) in Ar-sparged, stirred cultures (15-liter scale) at 72° C. with pH control; and (iii) in Ar-sparged, stirred cultures at 94° C. with a pH control (15-liter scale) followed by rapid cooling of the culture to 72° C. within 10 min (cold shock).

The recombinant strains of P. furiosus were grown at 98° C. and at 72° C. in closed, static cultures without a pH control. The ΔpdaD and LAC strains grew at 98° C. to comparable cell densities after 12 h (>10⁸ cells/ml, >50 μg protein/ml), but at 72° C. growth was still very poor even after 45 h (<10⁸ cells/ml, <20 μg protein/ml). Interestingly, cells changed their morphology at 72° C., becoming light refracting, and larger (up to 5 μm), in accordance with the upregulation of the expression of a wide range of genes, including those encoding numerous membrane-bound proteins (Weinberg et al., 2005. J. Bacteriol. 187:336-348). To determine whether recombinant protein production was successful, the cells were lysed by sonication, and the activity of NAD-dependent lactate dehydrogenase (LDH) was determined in cell-free extracts at 75° C. The activity of NAD-dependent glutamate dehydrogenase (GDH), a well-characterized cytoplasmic enzyme of P. furiosus (Adams, 1993. Annu. Rev. Microbiol. 47:627-658), was used as a control. The wild-type, ΔpdaD, and LAC strains had comparable specific activities of GDH when grown at 72° C. (0.09 to 0.11 U mg⁻¹) and when grown at 98° C. (0.14 to 0.27 U mg⁻¹). LDH activity was not detected (<0.05 U mg⁻¹) in cell-free extracts of any strain grown at 98° C. or in extracts of cells of the wild-type and parent strains grown at 72° C. However, extracts of the LAC strain grown at 72° C. had high LDH activity (1.8±0.1 U mg⁻¹). C. bescii ldh is the first bacterial gene to be expressed and to yield an active enzyme in P. furiosus (FIG. 1A). Remarkably, the specific activity of LDH in P. furiosus was comparable to that measured in cell-free extracts of cellobiose-grown C. bescii (2.5±0.7 U mg⁻¹; FIG. 1B), conditions under which C. bescii produces lactate as the major metabolic product. Moreover, while lactate was not detected (<20 μM) in the growth medium of any of the P. furiosus strains grown at 98° C. or in the wild-type and parent strains grown at 72° C., the medium of the LAC strain contained 0.47±0.14 mM lactate (FIG. 1C).

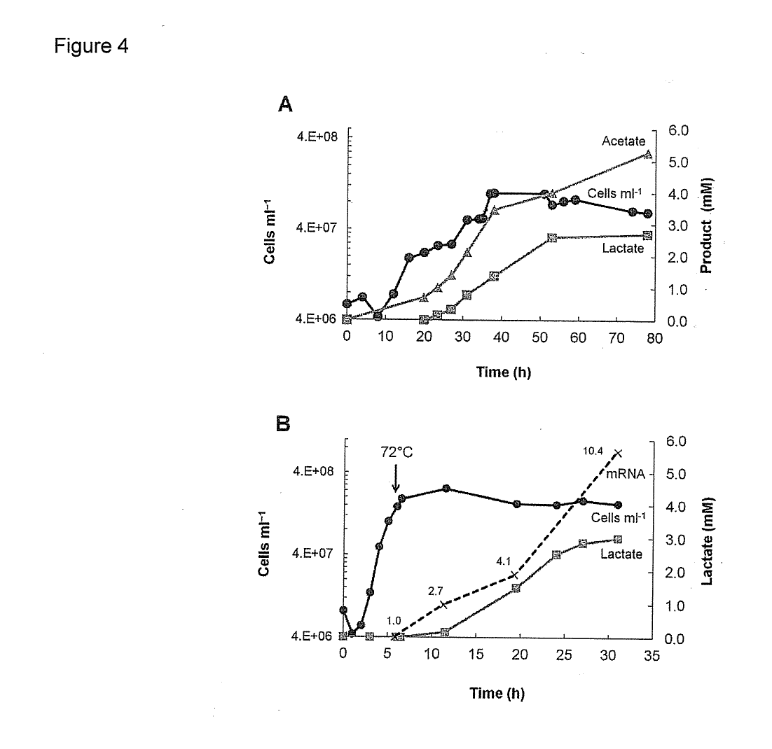

Growth of the P. furiosus LAC strain at 72° C. was scaled up in a stirred, pH-controlled fermentor (15 liters), conditions under which good growth of P. furiosus is obtained even at this low temperature (Weinberg et al., 2005. J. Bacteriol. 187:336-348). The organism reached a maximum cell density after approximately 50 h (1 10⁸ ml⁻¹, 60 μg ml⁻¹) (FIG. 4A) and remained stable in stationary phase for a further 28 h. The specific activity of LDH in the cell extract was the same as that measured in the small-scale cultures and remained unchanged (2.0±0.4 U mg⁻¹) in exponential (at 38 h [FIG. 4A]), early stationary (at 59 h), and late stationary (at 78 h) growth phase. Consequently, the amount of lactate produced paralleled the cell density. This reached a concentration near 3 mM in stationary phase, which was approximately half of the concentration of acetate that was produced (FIG. 4A). Therefore, we conclude that the P. furiosus LAC strain is robust and can be cultivated in large volumes with a specific LDH activity comparable to that measured in uncontrolled batch cultures but with higher yields of both total protein and lactate.

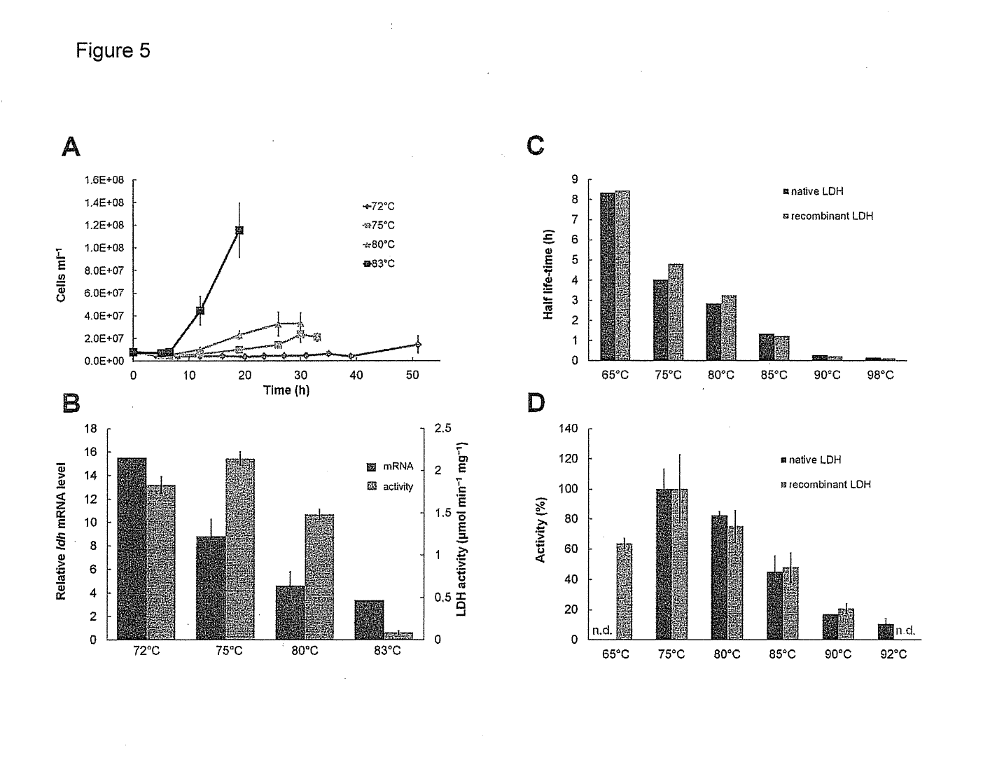

LDH activity and transcription of the C. bescii LDH gene were measured in the P. furiosus LAC strain over the growth temperature range from 72 to 83° C. Transcription of the ldh gene from C. bescii (Cbes-ldh) is controlled by the P_{cipA} promoter, and the corresponding CipA protein was reported previously to be produced at 72° C. (Weinberg et al., 2005. J. Bacteriol. 187:336-348), although no data are available on its expression at other temperatures. While the growth rate of P. furiosus LAC drastically increases with increasing temperature, the highest relative Cbes-ldh mRNA level was found in cultures grown at 72° C. (FIG. 5A). In addition, the highest specific LDH activities were detected in cultures grown in the 72 to 75° C. range (FIG. 5B). Obviously, the promoter induces transcription, and this leads to protein production at those low temperatures and to reasonable amounts and activities of the recombinant protein, even though the culture itself exhibited relatively poor growth (FIG. 5A).

To confirm that producing C. bescii LDH in P. furiosus at 72° C. was comparable to producing the enzyme in C. bescii, we determined the properties of the recombinant LDH produced in P. furiosus with those of the native LDH produced in C. bescii (FIGS. 5C and D). Both forms of the enzyme had temperature optima near 75° C., close to the optimal growth temperature of C. bescii (Yang et al. 2009. Appl. Environ. Microbiol. 75:4762-4769), and both fauns had only barely detectable activity above 90° C., which is above the maximal growth temperature of C. bescii (90° C.). Moreover, both forms of the enzyme had a relatively long half-life of about 5 h at the temperature optimum (75° C.). Such stability is comparable to that of the most thermostable LDH previously reported, the enzyme from Thermotoga maritima, an organism that has growth properties similar to that of C. bescii (Tₒₚₜ of 80° C.) (Ostendorp et al., 1996. Protein. Sci. 5:862-873).

In terms of temperature-dependent bioprocessing, a useful approach would be to grow P. furiosus to a high cell density under conditions that are nearly optimal for growth in the absence of heterologous gene expression and then cold shock the culture for bioproduction generation as a result of heterologous gene expression. The LAC strain was grown at 94° C., conditions known not to lead to detectable C. bescii LDH activity or detectable amounts of ldh mRNA, to a cell density of 2 10⁸ ml⁻¹, and the culture was rapidly cooled to 72° C. (over 10 min). At this point, lactate could not be detected in the culture medium. However, 5 h after the switch, mRNA corresponding to C. bescii ldh was detected and lactate was measured in the growth medium (FIG. 4B). Moreover, the concentration of both ldh mRNA (relative to the level of the gamma subunit of the constitutively expressed pyruvate-ferredoxin oxidoreductase) and lactate increased over the following 25 h (FIG. 4B), leading to the production of approximately 3 mM lactate. Cells contained C. bescii LDH with a specific activity of 1.9±0.6 U mg⁻¹. The latter value is comparable to those determined with cells grown in batch culture at 72° C. (FIG. 1B), showing that cold shock bioproduct generation is a valid experimental approach.

### Discussion

We have demonstrated that a microorganism (in this case, from the domain Archaea) that grows optimally near 100° C. transcribes mRNA and produces the corresponding enzyme, LDH, from another microorganism (in this case, from the domain Bacteria) that grows optimally at 78° C. but does so only under the conditions where the foreign protein shows significant catalytic activity, namely, below 80° C. The activity of the heterologously expressed LDH in P. furiosus might be the result of processes at both the RNA and protein level. First, the relative ldh mRNA level increased due to the cold-induced promoter, with an upregulation about 10-fold at 72° C. Although cold-responsive promoters have been previously reported in mesophilic bacteria, they were utilized to facilitate protein folding at low temperature (reference Jana and Deb, 2005. Appl. Microbiol. Biotechnol. 67:289-298 and references therein) rather than to exploit temperature induction for biotechnological purposes such as biofuel production. Second, the stability of the protein and its activity decreased with increasing temperature above 80° C. Interestingly, only two LDHs have been previously characterized from thermophiles, and they are homooligomeric enzymes (Ostendorp et al., 1996. Protein. Sci. 5:862-873, Zhou and Shao, 2010. Biochemistry (Mosc), 75:526-530). The finding that C. bescii LDH produced in P. furiosus and C. bescii were similarly thermostable suggests that the P. furiosus version was correctly assembled into its multimeric form.

Recombinant production of the C. bescii LDH represents the first bacterial protein to be expressed in a hyperthermophilic microorganism from the domain Archaea and one of the first heterologously expressed proteins in archaea in general (Matsumi et al., 2007. J. Bacteriol. 189:2683-2691, Takemasa et al., 2011. Appl. Environ. Microbiol. 77:2392-2398, Lessner et al., 2010. mBio, 1:e00243-10). It provides interesting options for the future production of other bacterial proteins, particularly ones involved with lignocellulosic biomass degradation, since an archaeon that can degrade crystalline cellulose has yet to be reported (Barnard et al., 2010, Environ. Technol. 31:871-888, Blumer-Schuette et al., 2008. Curr. Opin. Biotechnol. 19:210-217). Indeed, the lactate-producing strain described here offers a potential platform to enhance the temperature limit for lactate production from lignocellulosic substrates, a process of industrial interest (Wang et al., 2011. Proc. Natl. Acad. Sci. U.S.A. 108:18920-18925).

P. furiosus has therefore been metabolically engineered to change its end products of fermentation without the need for the addition of any chemical inducer, and thus any indirect impact on its metabolism or the accumulation of inducer products. Moreover, we demonstrate that temperature is an effective means of regulation even using cells grown rapidly to high cell density, particularly since the corresponding mRNA, enzyme activity, or product (lactate) could not be detected until the temperature was lowered. The unusual cold shock response of P. furiosus could be a powerful tool for biotechnological applications.

### Example 2

Metallosphaera sedula is an extremely thermoacidophilic archaeon that grows heterotrophically on peptides, and chemolithoautotrophically on hydrogen, sulfur, or reduced metals as energy sources. During autotrophic growth, CO2 is incorporated into cellular carbon via the 3-hydroxypropionate/4-hydroxybutyrate cycle (3HP/4HB). To date, all steps in the pathway have been connected to enzymes encoded in specific genes, except for the one responsible for ligation of coenzyme A (CoA) to 4-hydroxybutyrate (4HB). While several candidates for this step have been identified through bioinformatic analysis of the M. sedula genome, none have been shown to catalyze this biotransformation. Here, transcriptomic analysis of cells grown under strict H2-CO₂ autotrophy uncovered two additional candidates, encoded in Msed_{—}0406 and Msed_{—}0394. Recombinant versions of these enzymes catalyzed the ligation of CoA to 4HB, with similar affinities for 4HB (Km values of 1.9 and 1.5 mM for Msed_{—}0406 and Msed_{—}0394, respectively), but with different rates (1.69 and 0.22 μmol×min×mg⁻¹ for Msed_{—}0406 and Msed_{—}0394, respectively). Neither Msed_{—}0406 nor Msed_{—}0394 have close homologs in other Sulfolobales, although low sequence similarity is not unusual for acyl-adenylate forming enzymes. The capacity for these two enzymes to use 4HB as a substrate may have arisen from simple modifications to acyl-adenylate forming enzymes. For example, a single-amino acid substitution (Trp424 to Gly) in the active site of the acetate/propionate synthetase (Msed_{—}1353), an enzyme that is highly conserved among the Sulfolobales, changed its substrate specificity to include 4HB. The identification of the 4-HB CoA synthetase now completes the set of enzymes comprising the 3HP/4HB cycle.

### Experimental Procedures

Growth of M. sedula in a Gas Intensive Bioreactor

M. sedula (DSMZ 5348) was grown aerobically at 70° C. in a shaking oil bath (90 rpm) under autotrophic or heterotrophic conditions on DSMZ medium 88 at pH 2. Heterotrophically-grown cells were supplemented with 0.1% tryptone. Cell growth was scaled up from 300 ml in sealed one liter bottles (see previous work (Auernik and Kelly, 2010, Appl. Environ. Microbiol., 76:931-935)) to 2 liters in a stirred bench-top glass fermentor (Applikon), also on DSMZ medium 88 (pH 2) at 70° C., and agitated at 250 rpm. Two separately regulated gas feeds were used, such that flow rates were held constant for all conditions at 1 ml/min for the hydrogen/CO2 gas mixes (composition varied) and 100 ml/min for air (composition—78% N2, 21% O₂, 0.03% CO2). For the autotrophic, carbon-rich (ACR) condition, the gas feed contained H2 (80%) and CO2 (20%); for the autotrophic carbon-limited (ACL) condition the feed was changed to H2 (80%) and N2 (20%); for the heterotrophic condition (HTR), the medium was supplemented with 0.1% tryptone and the gas feed composition was N2 (80%) and CO2 (20%). Tandem fermentors were run simultaneously with the same inoculum to generate biological repeats. The tandem fermentors were started at the same time with the same seed inoculum, were used to grow M. sedula inside of a chemical fume hood. A solenoid valve on the H₂/CO₂ tank provided passive “fail-safe” operation by cutting off the flow of flammable gas in the event of hood failure. Cells were harvested at mid-exponential phase by rapid cooling with dry ice and ethanol, and then centrifuged at 6,000×g for 15 min at 4° C.

M. sedula Oligonucleotide Microarray Transcriptional Response Analysis

A spotted whole-genome oligonucleotide microarray, based on 2,256 protein-coding open reading frames (ORFs), was used, as described previously (Auernik et al., 2008, Appl. Environ. Microbiol. 74:7723-7732). Total RNA was extracted and purified (RNeasy; Qiagen), reverse transcribed (Superscript III; Invitrogen), re-purified, labeled with either Cy3 or Cy5 dye (GE Healthcare), and hybridized to the microarray slides (Corning). Slides were scanned on a GenePix 4000B Microarray Scanner (Molecular Devices, Sunnyvale, Calif.), and raw intensities were quantitated using GenePix Pro v6.0. Normalization of data and statistical analysis were performed using IMP Genomics 5 (SAS, Cary, N.C.). In general, significant differential transcription was defined to be relative change at or above 2 (where a log 2 value of ±1 equals a twofold change) with significance values at or above the Bonferroni correction; for these data, this was 5.4 (equivalent to a p-value of 4.0×10⁶). Microarray data are available through the NCBI Gene Expression Omnibus (GEO) under accession number GSE39944.

Enzyme Assays for 4-Hydroxybutyrate-CoA Synthetase

Two assays were used to measure ligase activity, one spectrophotometric and one using high-performance liquid chromatography (HPLC). A discontinuous assay was used to measure substrate-dependent disappearance of CoA at 75° C. The reaction mixture (600 μl) contained 100 mM MOPS-KOH (pH 7.9), 5 mM MgCl2, 2.5 mM ATP, 0.15 mM CoA, and purified enzyme. At each time point, 80 μl of reaction mixture was added to 80 μl cold 5,5′-dithiobis-(2-nitrobenzoic acid) (DTNB). A time point (0 min) was taken before heating. The reaction mixture was incubated for 2 min at 75° C., followed by addition of substrate. Additional time points were taken at 30, 60, 90, 120, and 180 sec after addition of substrate. Absorbance was measured at 412 nm to determine free CoA concentration, based on the concentration of 2-nitro-5-thiobenzoate dianion (NTB2-) (ε412=14,150 M⁻¹ cm⁻¹)(Hawkins et al., 2011, ACS Catal. 1:1043-1050, Riddles et al., 1983, Methods in Enzymology 91:49-60). Enzyme kinetics were determined by varying the concentration of the acyl-CoA substrate from 0.05 mM to 12 mM, while the other substrate concentrations were held constant. Formation of the CoA ester was also confirmed using HPLC (Waters). The reaction mixture (0.15 ml) contained 100 mM potassium phosphate (pH 7.9), 10 mM MgCl2, 2 mM ATP, 0.5 mM CoA, 10 mM substrate, and purified enzyme. The reaction was incubated for 3 min at 75° C., quenched with 15 μl 1M HCl, filtered with a 10 kDa spin column (Amicon YM-10) to removed the protein, and loaded onto a reversed-phase C18 silica-based column (Shodex C18-4E,4.6 250 mm). The mobile phase was 50 mM sodium phosphate buffer (pH 6.7) with 2% methanol.

Heterologous Expression of M. sedula Genes in E. coli

M. sedula genes encoding acyl-CoA synthetases were amplified from genomic DNA using primers synthesized by Integrated DNA Technologies (Coralville, Iowa). Msed_{—}0394 and Msed_{—}0406 were ligated into pET46-Ek/LIC, while Msed_{—}1353 was ligated into pET21b using NdeI and XhoI restrictions sites. All constructs were designed to express with an N-terminal His6-tag. Plasmids containing gene inserts were cloned into Novablue GigaSingles E. coli competent cells and selected by growth on LB-agar supplemented with ampicillin (100 μg/ml). Plasmid DNA was extracted using a QIAprep Spin Miniprep kit. Sequences were confirmed by Eton Biosciences, Inc. (Durham, N.C.). For protein expression, the plasmids were transformed into E. coli Rosetta 2 (DE3) cells and selected by growth on LB-agar, supplemented with ampicillin (100 μg/ml) and chloramphenicol (50 μg/ml). Cells harboring the recombinant plasmid were induced with IPTG (final concentration 0.1 mM) at OD600 0.4-0.6 and cultured for three hours before harvest.

Purification of Recombinant Proteins

Cells were harvested by centrifugation at 6,000 g for 15 min at 4° C. and then re-suspended in lysis buffer (50 mM sodium phosphate, 100 mM NaCl, 0.1% NP·40, pH 8.0) containing DNase and lysozyme at final concentrations of 10 and 100 μg/ml, respectively. Cells were lysed with a French Press (two passes at 18,000 psi) and the lysate was centrifuged at 22,000 g for 15 min at 4° C. to removed insoluble material. Soluble, cellfree extract was heated to 65° C. for 20 min to precipitate mesophilic proteins. Streptomycin sulfate (1% w/v) was added to precipitate nucleic acids, followed by a one hour incubation at 4° C. A final centrifugation was performed at 22,000 g for 15 min at 4° C. to collect the soluble, heat treated cell-free extract, which was sterile filtered (0.22 μm) and purified using a 5 ml HisTrap™ nickel column (GE Healthcare). Proteins were bound to the HisTrap™ column using binding buffer (50 mM sodium phosphate, 500 mM NaCl, 20 mM imidazole, pH 7.4) and eluted using elution buffer (50 mM sodium phosphate, 500 mM NaCl, 300 mM imidazole, pH 7.4). SDS-PAGE was then performed on the IMAC fractions to qualitatively determine the purity of the protein before further purification. Chromatography fractions containing the protein were concentrated and exchanged into phosphate buffer (50 mM potassium phosphate, 150 mM NaCl, pH 7.0) using an Amicon YM10 (Millipore) centrifugal filter membrane, centrifuged at 4000 g and 4° C. To quantify the amount of protein, a Bradford assay was performed on the concentrated IMAC fractions using known serial dilutions of bovine serum albumin (BSA) by taking absorbance readings at 595 nm. Protein was further purified using a Superdex 200 10/300 GL (GE Healthcare) gel filtration column. The proteins were eluted from the gel filtration column using elution buffer (50 mM potassium phosphate, 150 mM NaCl, pH 7.0). Proteins were dialyzed into 100 mM MOPSKOH (pH 7.9) and either stored at 4° C. or mixed with glycerol to 20% and stored at −80° C.

Site-Directed Mutagenesis of Msed_{—}1353

Msed_{—}1353 was mutated with the GENEART® Site-directed mutagenesis system (Life Technologies), using AccuPrime™ Pfx polymerase. Mutagenesis primers were designed to change W424 to glycine (Primer 1-5′-CCCTTTGGTAGCACTTGGGGAATGACTGAAACTGG, SEQ ID NO:41; Primer 2—reverse compliment of Primer 1). Plasmids with Msed_{—}1353-G424 were cloned into Novablue GigaSingles E. coli competent cells and selected by growth on LBagar supplemented with ampicillin (100 μg/ml). Sequences were confirmed by Eton Biosciences Inc (Durham, N.C.).

Structural Analysis of Acyl-CoA Synthetases

Three-dimensional structural models for M. sedula acyl-CoA synthetases were made using the ITASSER online server (Berg, 2011, Appl. Environ. Microbiol. 77:1925-1936, Berg et al., 2010, Nat. Rev. Microbiol. 8:447-460, Roy et al., 2010, Nat. Protoc., 5:725-738). All structures were generated using the Protein Data Base entry for S. enterica Acs (STM4275, 1PG4) as a template for alignment. Amino acid sequence alignments were generated using Chimera by superposition of I-TASSER 3D structural models.

Materials

Plasmid vectors and strains were obtained from Novagen (San Diego, Calif.) and Stratagene (La Jolla, Calif.). Chemicals, devices, and reagents were obtained from Fisher Scientific (Pittsburgh, Pa.), ACROS Organics (Geel, Belgium), Sigma Chemical Co. (St. Louis, Mo.), New England Biolabs (Ipswich, Mass.), Qiagen (Valencia, Calif.), Millipore (Billerica, Mass.) and Invitrogen (Grand Island, N.Y.). Gases were purchased from Airgas National Welders (Charlotte, N.C.). Protein purification columns were obtained from GE Healthcare (Piscataway, N.J.). The Bradford Assay reagent was obtained from Bio-Rad (Hercules, Calif.). Site-directed mutagenesis kit was obtained from Invitrogen (Life Technologies).

### Results

Metallosphaera sedula Autotrophic Growth is Hydrogen-Limited

In order to explore the optimal growth conditions for H2-CO2 autotrophy in M. sedula, a fermentation system was designed to allow controlled definition of the gas feed. Previous autotrophic work with M. sedula was done in batch cultures in an orbital shaking bath at 70° C. (Berg, 2011, Appl. Environ. Microbiol. 77:1925-1936, Berg et al., 2007, Science, 318:1782-1786, Alber et al., 2008, J. Bacteriol. 190:1383-1389, Hugler et al., 2003, Arch. Microbiol. 179:160-173, Auernik and Kelly, 2010, Appl. Environ. Microbiol., 76:931-935). In that case, gas-fed cultures were grown by replacing the air in a sealed volume with a gaseous mixture of a known composition. Mass transfer of H2, CO2, and O2 into the culture medium was limited to diffusion across the vaporliquid interface. Gas limitation presumably affected these cultures, and led to sub-optimal growth, as evidenced by the slow doubling time that resulted for M. sedula under these conditions (td=11 to 13 h).

In order to grow M. sedula autotrophically with more optimal delivery of gaseous substrate to the liquid medium, a semi-continuous fermentation system was developed using a 3 L bioreactor. The system was modified to have two separate gas feeds that sparged directly into the media (sparging stone—2 μm pore size). Microbubble sparging stones were used to promote dissolution of sparingly soluble gases, in particular H2. The bioreactor and console were situated inside a modified fume hood, with an airflow monitoring system in place to detect hood failure. Tandem fermentors were seeded with the same inoculum and run simultaneously to provide a biological repeat.

Growth of M. sedula in an aerobic, autotrophic fermentation system was expected to be H2-, and not O2-limited. Below saturating conditions, growth rates varied according to the amount of H2 fed to the culture. For high H2 supply rates (i.e., 30 ml/min), the growth rates were comparable to the fastest growth rates previously observed under heterotrophy (td=4.8 h); concomitantly, the culture reached a cell density of 2 109 cells/ml. the highest observed under autotrophic conditions. At a H2 supply rate of 15 ml/min, the growth rate slowed (td=6 h) although the final density was comparable to the 30 ml/min case (1.5 109 cells/ml). A 30-fold reduction in H2 flow rates (1 ml/min) caused the growth rate to decrease by half (td=9.7 h) and the cells to enter stationary phase at 8 10⁸ cells/ml.

A similar trend emerged in response to limiting levels of CO2. When CO2 was supplemented in the gas feed (referred to here as “rich” autotrophy), the growth rate was faster that observed for cells grown with air as the only source of CO2 (td=6.8 h vs. 9.4 h, respectively). The growth rate for heterotrophically grown cells (td=6.7 h) was comparable to the “rich” autotrophy condition. This suggests that, under the “rich” autotrophy condition, the cells were not limited by any one particular gaseous substrate and were doubling at or near their maximal rate. The decrease in growth rate for the carbon-limited autotrophy arises from the limiting amounts of CO2 available in the medium.

Optimized H2-CO2 Autotrophy Conditions Led to Enhanced Transcriptomic Response

The optimized autotrophic growth conditions enhanced the global transcriptional response compared to previous work (Berg et al., 2007, Science, 318:1782-1786, Huber et al., 2008, Proceedings of the National Academy of Sciences, U.S.A, 105:7851-7856, Auernik and Kelly, 2010, Appl. Environ. Microbiol., 76:931-935). Of the 2293 protein coding genes in the 2.2 kb M. sedula genome, nearly half (984 genes) exhibited changes in transcription (either up- or down-regulation) of two-fold or greater, when comparing heterotrophy (HTR) to the autotrophic carbon-limited (ACL) condition (See Table 3). The number of genes that were differentially transcribed was twice as high as previously observed (Berg et al., 2010, Nat. Rev. Microbiol. 8:447-460, Auernik and Kelly, 2010, Appl. Environ. Microbiol., 76:931-935), which could be attributed to the refined conditions for autotrophic growth. Also, in the experiments reported here, it should be mentioned that the improved sensitivity of new equipment used for scanning microarray slides improved the resolution and dynamic response.

TABLE 3
Enhanced Transcription Response for M. sedula Autotrophy

| | ACL-ACR | ACL-HTR | ACR-HTR | A-H (8) |
|---|---|---|---|---|
| # of genes | 52 | 467 | 433 | 229 |
| UP-regulated | | | | |
| (2-fold or more) | | | | |
| # of genes | 124 | 517 | 464 | 252 |
| DOWN-regulated | | | | |
| (2-fold or more) | | | | |

Overall, the global transcriptional changes were extensive. Transcripts for the characteristic enzymes of the 3HP/4HB pathway were significantly up-regulated on ACL-HTR. For example, the genes encoding α- and β-subunits of acetyl-CoA/propionyl-CoA carboxylase (Msed_{—}0147-0148), were up-regulated 18- and 29-fold, respectively, while the 4-hydroxybutyryl-CoA dehydratase gene (Msed_{—}1321), was upregulated 27-fold. Hydrogenases and hydrogenase assembly and maturation proteins in both the cytosolic hydrogenase operon (Msed_{—}0921-0933) and the membrane-bound hydrogenase operon (Msed_{—}0947-0950) were both highly up-regulated on ACL-HTR, from 3- to 47-fold higher.

New Candidates for 4-Hydroxybutyrate-CoA Synthetase Identified from Refined Transcriptomic Data

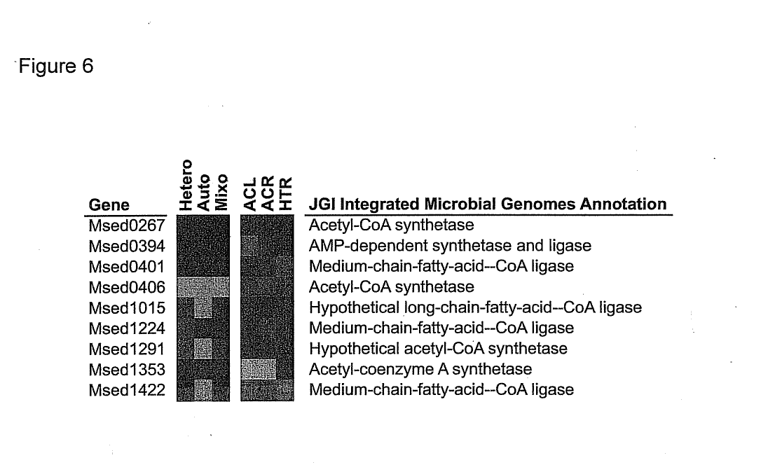

The refined transcriptomic data provided new insights into the putative candidates for 4-hydroxybutyrate-CoA synthetase (FIG. 6). Based on bioinformatic analysis, there are nine candidate genes encoding acyl-CoA synthetases (not including Msed_{—}456, which was confirmed as a 3HP-CoA synthetase). The high up-regulation of Msed_{—}1422 under autotrophy observed in this work is consistent with previous transcriptomic studies. On the basis of that work, Msed_{—}1422 was chosen for recombinant expression and testing (Berg, 2011, Appl. Environ. Microbiol. 77:1925-1936, Ramos-Vera et al., 2011, J. Bacteriol. 193:1201-1211, Estelmann et al., 2011, J. Bacteriol. 193:1191-1200). In the same study, recombinant Msed_{—}1291 and Msed_{—}1353 were also produced, which were chosen based on homology to a confirmed 4HB-CoA synthetase from Thermoproteus neutrophilus (Tneu_{—}0420). Both Msed_{—}1422 and Msed_{—}1291 showed no activity on acetate, propionate, 3HP, 3HB, 4HB, or crotonate. Msed_{—}1353 had activity only on acetate and propionate, but not 4HB. Thus, it appears that Msed_{—}1353 is a promiscuous acetate/propionate synthetase, while the substrate specificities of Msed_{—}1422 and Msed_{—}1291 remain unknown.

Among the other potential candidates that were annotated as acetate-CoA synthetases or mediumchain fatty acid-CoA synthetases (FIG. 6), most showed no transcriptional response, had average or low levels of transcription, or were clearly down-regulated under autotrophy. The new transcriptomic data were consistent with the expression of two previously unexamined candidates, Msed_{—}0406 and Msed_{—}0394, which are annotated as an acetyl-CoA synthetase (ACS) and AMP-dependent synthetase and ligase, respectively. Although Msed_{—}0406 and Msed_{—}0394 were both constitutively transcribed, with less than a two-fold change in transcription levels between the conditions tested, both of them were in the top 25% of the transcriptome. Both of these genes were, thus, selected for recombinant expression and activity assay, given that no other promising candidates for this step had emerged.

Kinetic Analyses of Msed_{—}0394 and Msed_{—}0406

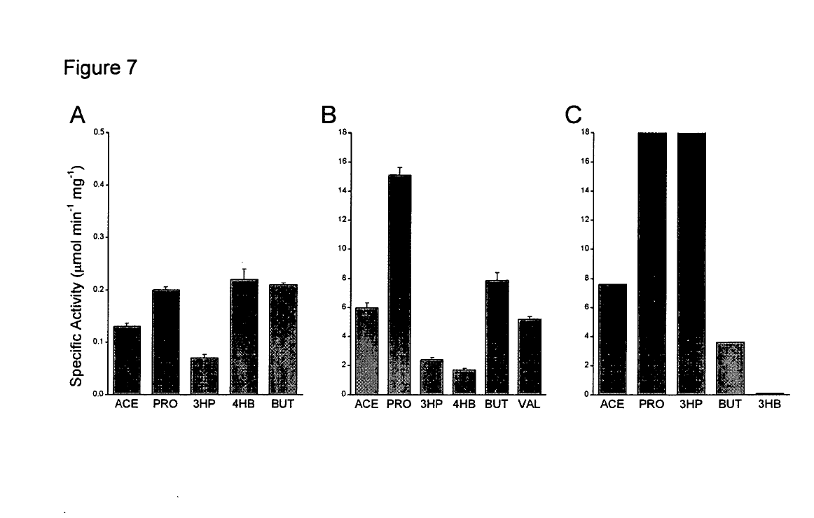

Recombinant fauns of Msed_{—}0394 and Msed_{—}0406 were produced in E. coli. For both enzymes, the production of 4HB-CoA from 4HB and CoA was confirmed using reversed-phase HPLC. Msed_{—}0394 and Msed_{—}0406 were active on a range of small organic acids. FIG. 7 shows the relative activity on different substrates for Msed_{—}0394, Msed_{—}0406, along with reported data for 3HP-CoA synthetase (Msed_{—}1456) for comparison (Berg et al., 2007, Science, 318:1782-1786, Alber et al., 2008, J. Bacteriol. 190:1383-1389, Estelmann et al., 2011, J. Bacteriol. 193:1191-1200). The activity of Msed_{—}0406 on all substrates was an order of magnitude higher than Msed_{—}0394, including 4HB (1.8 vs. 0.22 μmol min⁻¹ mg⁻¹). However, Msed_{—}0406 had much higher specificity for propionate (290 μM) than for 4HB (2000 μM). Msed_{—}0394 activity was much lower overall, with smaller differences in substrate specificity. On 4HB, Msed_{—}0406 was more catalytically efficient than Msed_{—}0394 (900 vs 150 s⁻¹ M⁻¹), suggesting that it was the most physiologically relevant enzyme. Comparison of Msed_{—}0406 on 4HB to reported data for Msed_{—}1456 on 3HP shows that the turnover number for Msed_{—}0406 is smaller (2 s⁻¹ and 23 s⁻¹, respectively) and the Michaelis-Menten constant is an order of magnitude larger for Msed_{—}0406 on 4HB (2000 mM) than for Msed_{—}1456 on 3HP (180 mM). Msed_{—}1456, therefore, has a much higher catalytic efficiency on 3HP (12.8 104 s⁻¹ M⁻¹) than Msed_{—}0406 has on 4HB (9 102 s⁻¹ M⁻¹).

Site-Directed Mutagenesis of Msed_{—}1353

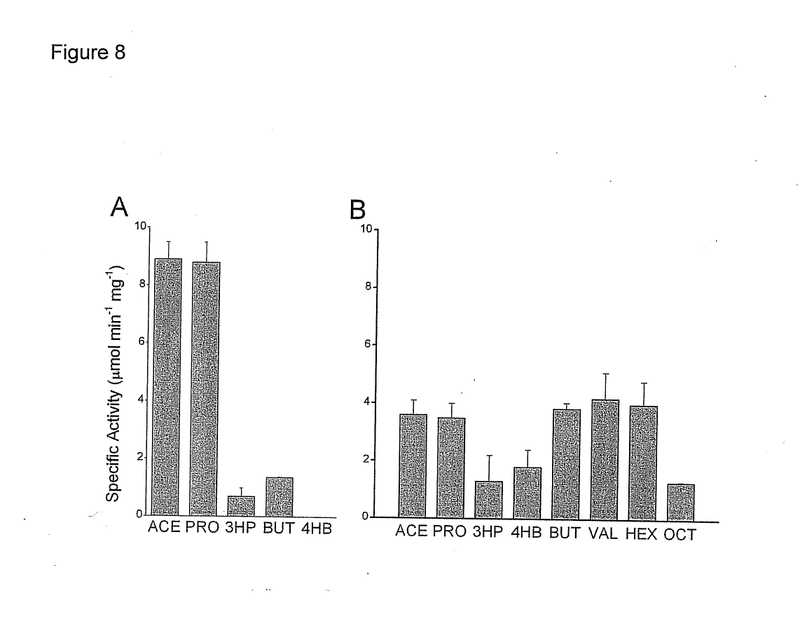

Msed_{—}1353, a highly conserved gene among the Sulfolobales, was previously reported to have activity only on acetate and propionate (Berg et al., 2007, Science, 318:1782-1786, Alber et al., 2008, J. Bacteriol. 190:1383-1389, Ramos-Vera et al., 2011, J. Bacteriol. 193:1201-1211, Hügler et al., 2003, Eur. J. Biochem. 270:736-744, Alber et al., 2006, J. Bacteriol. 188:8551-8559, Auernik et al., 2008, Appl. Environ. Microbiol. 74:7723-7732). Initial efforts to identify the unknown 4HB-CoA synthetase in M. sedula involved purification of native enzyme activity and analysis of multiple SDS-PAGE gel bands using mass spectrometry. Msed_{—}1353 was detected in these experiments and, based on the very large upregulation of Msed_{—}1353 under autotrophy, it was recombinantly produced to confirm its activity. Our results confirmed previous reports: Msed_{—}1353 had activity on acetate (8.9 mmol min⁻¹ mg⁻¹—100%) and propionate (99%), but also on 3HP (8%) and butyrate (16%). However, no activity was found on 4HB or longer organic acid substrates (see FIG. 8A).

Structural analysis of the binding pocket of Msed_{—}1353 revealed a conserved tryptophan residue, similar to that seen in acetate-CoA synthetase (ACS) from S. enterica (Berg et al., 2007, Science, 318:1782-1786, Riddles et al., 1983, Methods in Enzymology 91:49-60, Gulick et al., 2003, Biochemistry 42:2866-2873). This tryptophan forms the bottom surface of the binding pocket and limits the size of substrate that can be accommodated within the active site. To test the importance of this residue in determining substrate specificity, Trp424 in Msed_{—}1353 was mutated to a glycine to produce Msed_{—}1353-0424. The single substitution mutant (Trp424 to Gly) was predicted to contain a larger interior binding pocket for the hydrophobic end of the substrate. Accordingly, it showed a dramatic change in specificity (FIG. 8B). Activity for the mutant on acetate and propionate decreased by 60%, from 8.9 to 3.6 and 8.8 to 3.5 μmol min⁻¹ g¹, respectively. However, Msed_{—}1353-G424 also showed activity on C4-C8 substrates. The activity with 4HB (1.8 μmol min⁻¹ mg⁻¹) was similar to that measured with Msed_{—}0406, which is the leading candidate for catalyzing the physiological reaction. In fact, the catalytic efficiency of Msed_{—}1353-G424 on 4HB was 2200 M⁻¹ s⁻¹, twice that of Msed_{—}0406 (906 M⁻¹ s⁻¹) and more than ten times that of the second candidate, Msed_{—}0394 (160 M⁻¹ s⁻¹).

### Discussion

The semi-continuous gas-intensive bioreactor system developed here was successfully used to refine the transcriptional response of autotrophyrelated genes in M. sedula. This system provided better delivery of sparingly soluble gases and allowed more precise regulation of gas composition than headspace. At 70° C. and 1 atm, the solubility of oxygen and hydrogen are comparable (0.6 mM), while the solubility of carbon dioxide is about 20-fold higher (12 mM) (Auernik and Kelly, 2010, Appl. Environ. Microbiol., 76:931-935, Ramos-Vera et al., 2011, J. Bacteriol., 193:1201-1211, Wilhelm et al., 1977, Chem. Rev., 77:219-262). For these experiments, the low solubility of H2 was offset by the use of microbubbler sparing stones (2 mm pore size) to increase the gas phase surface area and increase delivery of H2 to the medium.

Stoichiometrically, at least four H2 molecules are required for every carbon atom fixed. Assuming that ATP generation requires the oxidation of two hydrogen molecules, then each turn of the cycle requires 10 molecules of hydrogen for every two molecules of carbon dioxide. As such, the limiting growth factor for M. sedula in a bioreactor is likely acquisition of the electron donor, in contrast to most aerobic microbial fermentation where acquisition of the final electron acceptor, oxygen, limits growth. In its natural environment, the picture may be somewhat different. Hydrogen measurements from the (largely anoxic) acidic hot springs at Yellowstone indicate that gaseous hydrogen may be quite abundant—with concentrations ranging between 10-300 nM (Auernik and Kelly, 2010, Appl. Environ. Microbiol., 76:931-935, Spear et al., 2005, Proc. Natl. Acad. Sci. U.S.A. 102:2555-2560). The source of this hydrogen gas is primarily geochemical; although the mechanism is not well understood, it probably arises from subsurface interaction of water with Fe[II] (Auernik et al., 2008, Appl. Environ. Microbiol. 74:7723-7732, Sleep, 2004, Proc. Natl. Acad. Sci. U.S.A. 101:12818-12823). For most subsurface environments, oxygen is probably limiting (Gold, 1992, Proc. Natl. Acad. Sci. U.S.A. 89:6045-6049). However, M. sedula was isolated from aerobic (surface) samples of a hot water pond at Pisciarelli Solfatara (Huber et al., 1989, Syst. Appl. Microbiol. 12:38-47). Thus both hydrogen and oxygen may be available in abundance for autotrophic growth.

The regulation of growth modes in M. sedula involves massive transcriptional changes between heterotrophic and autotrophic growth. Nearly half the genome (984 genes out of 2293) responded with transcriptional changes of 2-fold or greater when comparing heterotrophy to carbon dioxide limited autotrophy. Not much is known about the regulation strategies employed by archaea to control gene transcription, but between different forms of chemolithoautotrophy (reduced metals, H2, etc.) and heterotrophy, M. sedula can utilize a broad range of metabolic substrates for growth.

The missing step in the 3HP/4HB pathway has been the acyl-CoA synthetase that utilizes 4HB. Previous attempts to identify the gene that encodes this enzyme were unsuccessful, and the candidate enzymes had no activity on 4HB (Ramos-Vera et al., 2011, J. Bacteriol. 193:1201-1211). In this work, two previously unexamined synthetases from M. sedula, consistent with the new transcriptomic evidence, were recombinantly produced and characterized. Both Msed_{—}0394 and Msed_{—}0406 showed activity on 4HB as well as other small organic acids. Based on the lack of other synthetase candidates suggested by the transcriptomic analysis and previous biochemical evidence ruling out Msed_{—}1422 and Msed_{—}1291, we conclude that one or both of these enzymes are necessary for autotrophic growth in M. sedula.

Acetyl-CoA synthetases belong to the Class I superfamily of adenylate-forming enzymes that includes acyl- and aryl-CoA synthetases, the adenylation domains of non-ribosomal peptide synthetases (NRPSs), and firefly luciferase (Schmelz and Naismith, 2009, Current Opinion in Structural Biology 19:666-671). These enzymes use a two-step mechanism driven by ATP hydrolysis (Gulick, 2009, ACS chemical biology 4:811-827). Most acetyl-CoA synthetases have a limited substrate range. Archaeal acyl-CoA synthetases, which form a phylogenetic cluster distinct from other bacterial subgroups (Bräsen et al., 2005, Extremophiles 9:355-365), have been reported to exhibit broader substrate preferences. The acetyl-CoA synthetase from Pyrobaculum aerophilum can work on acetate, propionate, butyrate, and isobutyrate (Bräsen et al., 2005, FEBS Lett. 579:477-482); another acetyl-CoA synthetase from Archaeoglobus fulgidus was active on acetate, propionate, and butyrate (Ingram-Smith and Smith, 2007, Archaea 2:95-107). Both Msed_{—}0394 and Msed_{—}0406 were found to have activity on a broad range of small organic acid substrates of up to five carbons in length.

Activity of both purified Msed_{—}0394 and Msed_{—}0406 on 4HB was well above the reported activity measured in autotrophic cell extract (0.3 μmol min⁻¹ mg⁻¹) (Berg et al., 2007, Science, 318:1782-1786). It appears that Msed_{—}0406 is primarily a promiscuous propionate-CoA synthetase. Msed_{—}0394, by contrast, has nearly equal levels of activity on acetate, propionate, and 4-HB. Although the overall activity for Msed_{—}0394 is lower by comparison, the enzyme appears to have poor specificity and functions equally well on a range of small organic acids. By comparison, the homologous 4-HB-CoA synthetase from Thermoproteus neutrophilus (Tneu_{—}0420), an anaerobic archaeon with the DC/4HB carbon fixation cycle, was recombinantly produced and shown to have maximal activity on 4HB, followed by crotonate, acetate, 3HP, and 3HB (Ramos-Vera et al., 2011, J. Bacteriol. 193:1201-1211). The reported Km for Tneu_{—}0420 is about 3-fold lower than that found for Msed_{—}0406 (700 mM vs. 2000 mM), with comparable activity (1.6 vs. 1.8 mmol min-1 mg-1), which suggests that the catalytic activities on 4HB are also comparable.

It is likely that Msed_{—}0406 is more effective at catalyzing the ligation of CoA to 4HB in vivo than Msed_{—}0394. Perhaps, these enzymes have evolved from highly specific acetate/propionate synthetases to be sufficient for catalyzing the necessary reaction on 4HB for the 3HP/4HB fixation cycle. It is not clear why two synthetases would be required, or if both of them are necessary for autotrophic growth. However, they are so far the only ligases in M. sedula that have been shown to activate 4HB with CoA.

Genes with high homology to Msed_{—}0394 and Msed_{—}0406 exist in the genome of the closely related M. cuprina (67% and 73% amino acid identity, respectively), but it is less clear whether homologs exist in the genomes of other Sulfolobales, such as the Sulfolobus and Acidianus spp. Members of the acyl-adenylate forming enzyme family may share little identity or similarity in amino acid sequence apart from a few highly conserved core motifs (Ingram-Smith and Smith, 2007, Archaea 2:95-107). There are homologs of Msed_{—}0406 in other species of Sulfolobales that have 30-35% identity, and one homolog in S. acidocaldarius with 61% identity. But the effort to find the M. sedula 4HB-CoA synthetase has shown that substrate specificity cannot be inferred from amino acid sequence homology alone. However, the low homology of the M. sedula 4HB-CoA synthetase gene does stand out among all the other genes in the 3HP/4HB cycle, which have distinct homologs in Sulfolobus spp. that range from 50-80% identity.

Since 4HB is a metabolite unique to butyrate metabolism (Pryde et al., 2002, FEMS Microbiol. Lett. 217:133-139), including γ-aminobutyrate fermentation (Gerhardt et al., 2000, Arch. Microbiol. 174:189-199) and polyhydroxyalkanoate production (Valentin et al., 1995, Eur. J. Biochem. 227:43-60)), it is unlikely to have any other role in crenarchaeal metabolism outside of carbon fixation. Formation of 4HB from succinic semialdehyde in the 3HP/4HB pathway is thought to occur via a unique flavin adenine dinucleotide and [4Fe-4S] cluster-containing enzyme, 4-hydroxybutyryl-CoA dehydratase (4hbd).

Recent work with metabolic flux analysis has shown there is another exit route for carbon flux from the cycle through succinyl-CoA to succinate (Estelmann et al., 2011, J. Bacteriol. 193:1191-1200). In this study the authors estimate that ⅔ of the cycle carbon flux passes to succinate via succinyl-CoA or succinic semialdehyde, while ⅓ of the cycle carbon flux passes through the latter part of the cycle (via 4HB) to regenerate acetyl-CoA. Of course, this flux distribution may be highly dependent on growth conditions and could shift more to the 4HB leg depending on substrate availability.

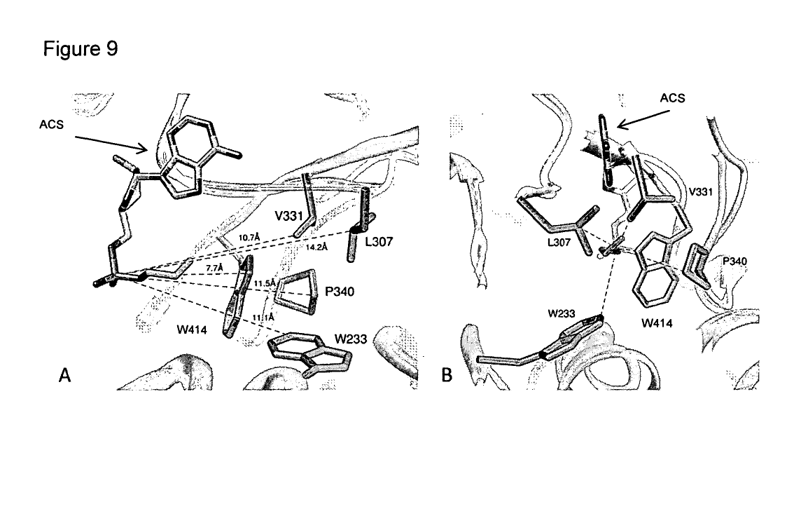

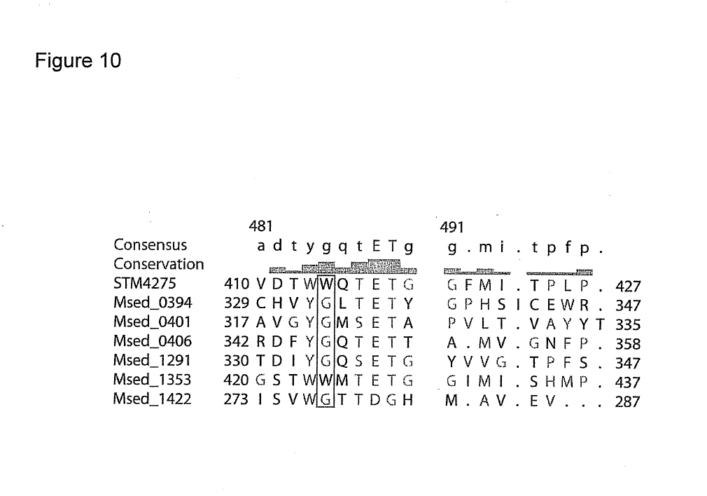

It is clear that all members of the Sulfolobales order have a homolog for 4hbd, and therefore should have a complete set of enzymes for carbon fixation. But, previous studies have been mixed as to whether Sulfolobus spp. are capable of autotrophic growth. Early reports on Sulfolobus acidocaldarius isolates claimed that they could grow chemolithoautotrophically on elemental sulfur (Brock et al., 1972, Arch. Microbiol. 84:54-68, Shivvers and Brock, 1973, J. Bacteriol. 114:706-710). Subsequent reports claim that neither S. solfataricus nor S. acidocaldarius can grow autotrophically on elemental sulfur alone (Grogan, 1989, J. Bacteriol. 171:6710-6719), although it is unclear whether they simply lost the ability to grow chemolithoautotrophically or were selected from what were originally mixed cultures (Kletzin et al., 2004, J. Bioenergetics and Biomembranes 36:77-91). Recent reports have shown autotrophic growth of S. metallicus on sulfur and S. tokodaii on both sulfur and iron (Bathe et al., 2007, Appl. Environ. Microbiol. 73:2491-2497). The only other member of the Sulfolobales that has been reported to grow on hydrogen is Acidianus ambivalens, a sulfur-reducing acidophile (Laska, 2003, Microbiol. 149:2357-2371). Genes encoding for hydrogenase and maturation enzymes with homology to M. sedula hydrogenase genes are present in one strain of S. islandicus (HVE10/4), but this is predicted to be involved in anaerobic fermentation (Guo et al., 2011, J. Bacteriol. 193:1672-1680). Clearly, some Sulfolobus spp. must have a functional carbon fixation pathway, but others seem to possess an incomplete or non-functional pathway. It may be that the CoA-activating ligase that can operate on 4HB is essential for complete cycle function, and loss of 4HB-CoA synthetase activity renders the carbon fixation cycle inoperable. To investigate the issue of substrate specificity, de novo structural predictions of M. sedula acyl-CoA synthetases with crystal structures were compared with other known synthetases, including acetyl-CoA synthetase from both S. enterica (Gulick et al., 2003, Biochemistry 42:2866-2873) and S. cerevisiae (Jogl and Tong, 2004, Biochemistry 43:1425-1431), and 4-chlorobenzonate-CoA synthetase from Alcaligenes sp. (Gulick et al., 2004, Biochemistry 43:8670-8679). The structure for ACS from S. enterica revealed that there are four residues that form the acetate binding pocket—Va1310, Thr311, Va1386, and Trp414 (Gulick et al., 2003, Biochemistry 42:2866-2873). The conserved tryptophan residue cuts the binding pocket short and precludes activity on longer substrates (FIG. 9). Extensive mutagenesis of binding pocket residues in yeast ACS showed that mutation of Trp416 to Gly416 was sufficient to lengthen the binding pocket to accommodate C4-C8 organic acids (Ingram-Smith et al., 2006, Biochemistry 45:11482-11490). Amino acid sequence alignments show that Msed_{—}1353 has a tryptophan in the same position (Trp424) (FIG. 10) and should, therefore, only work on acetate and propionate, a fact that has been confirmed biochemically (Ramos-Vera et al., 2011, J. Bacteriol. 193:1201-1211). Here, there was some activity with Msed_{—}1353 on 3HP and butyrate, but no activity on 4HB. Msed_{—}0394 and Msed_{—}0406 both have a glycine in this position, G333 and G346, respectively. However, the rest of the genes annotated as acyl-CoA synthetases in M. sedula also have a glycine in this position, so this glycine residue alone is not sufficient to indicate activity on C3-C5 unsubstituted linear organic acids. Both Msed_{—}1422 and Msed_{—}1291 were recombinantly expressed and showed to be inactive on C2-C4 linear organic acids (Ramos-Vera et al., 2011, J. Bacteriol. 193:1201-1211).

A mutant of Msed_{—}1353 with a glycine in place of the conserved tryptophan (Trp424 to Gly) was made by site directed mutagenesis and expressed in E. coli (Msed_{—}1353-G424). The native enzyme was active only on acetate and propionate, but the mutant showed activity on 3HP, 4HB, valerate, hexanoate, and even octanoate (FIG. 8). The activity was just as high on C5-C8 substrates as on acetate and propionate, but lower on 3HP and 4HB. This suggests that the polar hydroxyl group destabilizes the interaction between the substrate and the residues of the enlarged binding pocket. A similar trend is evident with Msed_{—}0406 (FIG. 7). However, Msed_{—}0394 has nearly equal levels of activity on propionate, butyrate, and 4HB, suggesting that it can stabilize the hydroxyl group on 4HB better than that of 3HP. Similarly, Msed_{—}1456, which catalyzes the ligation of CoA to 3HP in the 3HP/4HB pathway, has equal activity on propionate and 3HP, and therefore might have residues in the active site that help stabilize the hydroxyl group of 3HP.

In Msed_{—}1456, Va1386, which makes contacts with the g-carbon of the propyl moiety in the S. enterica ACS structure, is replaced with Asn³⁹⁰, whose polar amide nitrogen could hydrogen bond with the hydroxyl group of 3HP to stabilize substrate binding. As for Msed_{—}0406, both valine residues in the acetate binding pocket are replaced with alanine (Ala²⁴⁹ and Ala³²¹) and Thr³¹¹ is replaced with a lysine (Lys250). In Msed_{—}0394, all three of these residues are alanine (Ala²⁴⁰, Ala²⁴¹, and Ala³⁰⁹). Potential candidate residues for stabilizing the hydroxyl group of 4HB in Msed_{—}0394 include His³⁴¹ and Tyr³³⁸.

This work helps to close the gaps on the missing piece of the 3HP/4HB pathway in M. sedula. It is still unclear why only certain members of the Sulfolobales operate the 3HP-4HB cycle, but this may reflect the environmental history of specific species. Furthermore, along with other recent successes obtaining recombinant versions of difficult to produce enzymes from the pathway (Han et al., 2012, Appl. Environ. Microbiol., 78:6194-202), complete characterization of all cycle enzymes is near at hand. The information obtained for cycle function will be invaluable for the creation of a metabolically engineered platform capable of producing of chemicals and fuels from carbon dioxide (Hawkins et al., 2011, ACS Catal. 1:1043-1050).

### Example 3

Metabolically-engineered microorganisms can be utilized to produce a variety of products ranging from bulk chemicals and fuels to complex pharmaceutical molecules. The largest effort is currently in biofuel production from renewable plant biomass (Somerville et al., Science 329:790-792 (2010), Olson et al., Curr Opin Biotechnol 23:396-405 (2012), Steen et al., Nature 463:559-562 (2010)). Ethanol from corn fermentation and fatty acid methyl esters from edible oils and fats represent first generation biofuels, while next generation biofuels utilize cellulosic biomass as feedstocks and/or generate higher alcohols (Peralta-Yahya et al., Biotechnol J 5:147-162 (2010)). An alternative method for the microbial production of both fuels and chemicals that circumvents the overall low efficiency of both plant and algal photosynthesis is to use low potential reducing power, not from sugars in biomass, but from sources such as hydrogen gas, reduced metals or an electric current (Wackett, Curr Opin Biotechnol 22:388-393 (2011)). Moreover, such electron sources can potentially be used to reduce carbon dioxide directly to produce liquid fuels, or so-called electrofuels (Hawkins et al., ACS Catalysis 1:1043-1050 (2011)) or to produce industrial chemicals, or “electrochemicals”. However, while significant advances have been made in metabolically engineering microorganisms for fuel production (Peralta-Yahya et al., Biotechnol J 5:147-162 (2010), Shen et al., Appl Environ Microbiol 77:2905-2915 (2011), Connor et al., Curr Opin Biotechnol 20:307-315 (2009)), conferring the ability on a microorganism to utilize hydrogen and carbon dioxide to generate an industrial chemical has not been reported. Herein we have utilized a novel temperature-dependent approach (Example 1) to engineer a microorganism that grows on sugars optimally at 100° C. to also utilize carbon dioxide near 70° C. Hydrogen gas is used as the reductant to incorporate the carbon of carbon dioxide to produce 3-hydroxypropionic acid (3-HP), one of the top twelve industrial chemical building blocks used in the production of acrylic acid, acrylamide and 1,3-propanediol (Paster et al., Industrial bioproducts: today and tomorrow. US Department of Energy and Energetics Inc., Columbia, Md. (2004), Werpy et al., T. & Petersen, G. Top value added chemicals from biomass: volume 1—Results of screening for potential candidates from sugars and synthesis gas. Dept. of Energy, 102004-1992, (2004)). Furthermore, the metabolic burden of the engineered microorganism during chemical production from hydrogen and carbon dioxide is minimized by the strategic use of temperature.

### Results and Discussion

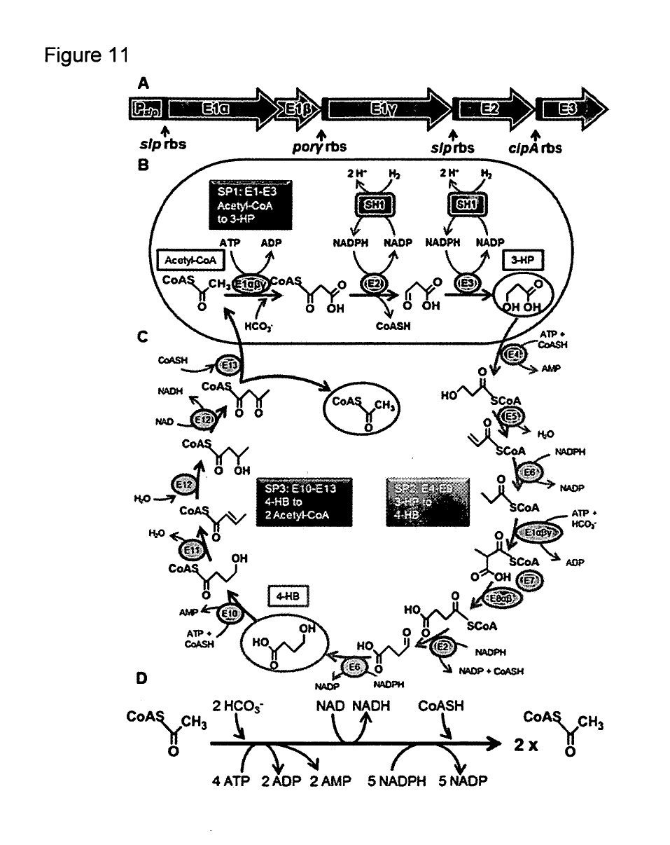

The hyperthermophilic archaeon Pyrococcus furiosus is an obligate heterotroph that grows optimally (Tₒₚₜ) at 100° C. by fermenting sugars to hydrogen, carbon dioxide and acetate (Fiala et al., Arch. Microbiol. 145 (1986)). It does not utilize carbon dioxide as a carbon source. A genetic system is available for P. furiosus based on a competent strain with a known sequence (Bridger et al., J Bacteriol 194:4097-4106 (2012)) that has allowed both homologous (Chandrayan et al., J Biol Chem 287:3257-3264 (2012), Hopkins et al., PLoS One 6:e26569 (2011)) and heterologous over-expression of genes (Example 1). A novel means of metabolic control was recently reported in P. furiosus that exploited the difference in the temperature dependence of host metabolism and the inserted foreign synthetic pathway (Example 1). For example, expression in P. furiosus of the gene encoding lactate dehydrogenase from a moderately thermophilic bacterium (Caldicellulosiruptor bescii, Tₒₚₜ 78° C.) resulted in temperature-dependent lactate formation (Example 1). Moreover, the engineered pathway is active near 70° C. when the host metabolism of P. furiosus is minimal at nearly 30° C. below its optimal temperature. Hence the host will require minimal maintenance energy and, as a result, minimal metabolic burden, while the engineered pathway that it contains is optimally active. This temperature-dependent approach for bioproduct generation has been used to express in P. furiosus genes encoding carbon dioxide fixation and 3-HP synthesis from the thermophilic archaeon Metallosphaera sedula (Tₒₚₜ 73° C.: (Berg et al., Nat Rev Microbiol 8:447-460 (2010))). The genes are the first part of the 3-hydroxypropionate/4-hydroxybutyrate pathway of M. sedula that consists of 13 enzymes (Ramos et al., J Bacteriol 193:1201-1211 (2011)). In one turn of the cycle, two molecules of carbon dioxide are added to one molecule of acetyl-CoA (C2) to generate a second molecule of acetyl-CoA (. 13C). The cycle can be divided into three sub-pathways (SP1-SP3) where SP1 generates 3-hydroxypropionate (3-HP) from acetyl-CoA and carbon dioxide, SP2 generates 4-hydroxybutyrate (4-HB) from 3-HP and carbon dioxide, and SP3 converts 4-HB to two molecules of acetyl-CoA. The reducing equivalents and energy for the pathway are supplied by NADPH and ATP, respectively (FIG. 11D). Notably, the 3-HP/4-HB pathway is purportedly more energetically efficient than carbon dioxide fixation by the ubiquitous Calvin cycle (Berg et al., Science 318:1782-1786 (2007)).

The first three enzymes of the Msed 3-HP/4-HB cycle are the SP1 pathway and together they produce 3-HP (FIG. 11B). The three enzymes are referred to here as E1 (acetyl/propionyl-CoA carboxylase, encoded by Msed_{—}0147, Msed_{—}0148, Msed_{—}1375), E2 (malonyl/succinyl-CoA reductase, Msed_{—}0709) and E3 (malonate semialdehyde reductase, Msed_{—}1993) (Berg et al., Science 318:1782-1786 (2007), Hugler et al., Eur J Biochem 270:736-744 (2003), Alber et al., J Bacteriol 188:8551-8559 (2006)). E1 carboxylates acetyl-CoA using bicarbonate and requires ATP. E2 breaks the CoA thioester bond and with E3, reduces the carboxylate to an alcohol with NADPH as the electron donor. E1 and E2 are bifunctional and are also involved in the SP2 part of the cycle (FIG. 11C). To demonstrate the concept, we expressed the M. sedula SP1 pathway in P. furiosus so that the organism could utilize carbon dioxide for the production of 3-HP using hydrogen as the electron donor. Hydrogen is utilized in P. furiosus by its cytoplasmic hydrogenase (SHI) that reduces NADP to NADPH (Ma and Adams, Method Enzymol 331:208-216 (2001)). SHI is extremely active even at 70° C. and a P. furiosus strain engineered to over-express the enzyme was previously developed (Chandrayan et al., J Biol Chem 287:3257-3264 (2012)).

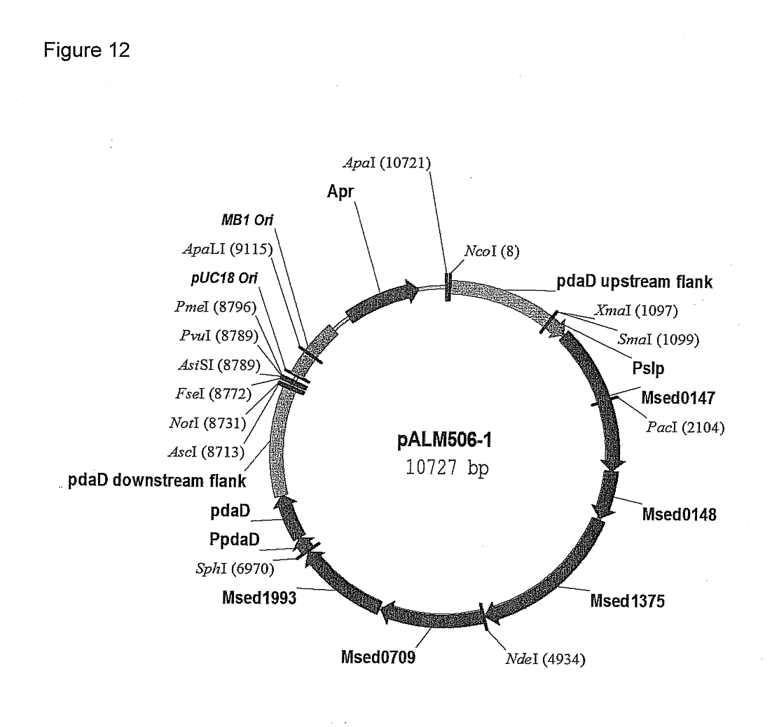

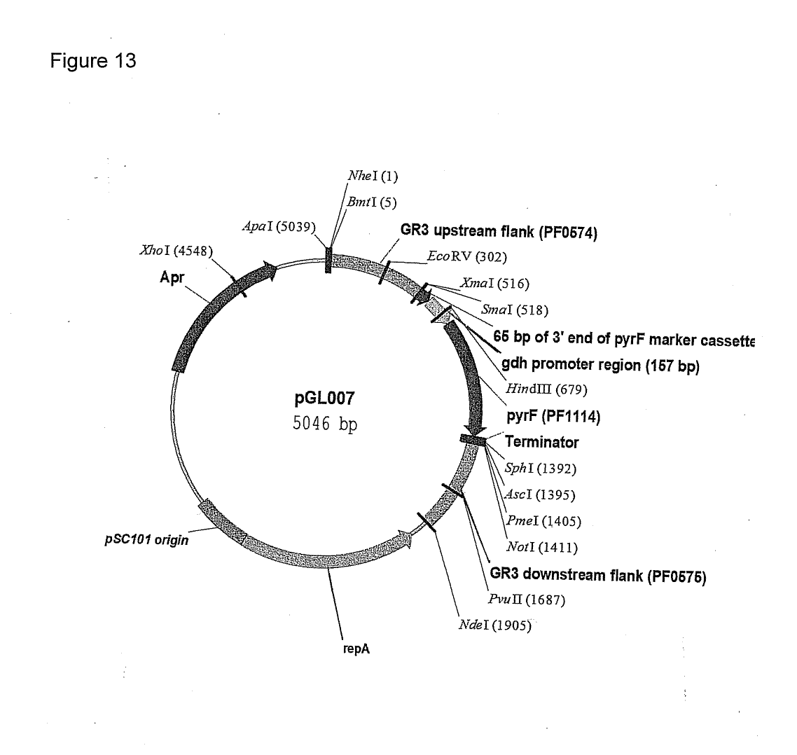

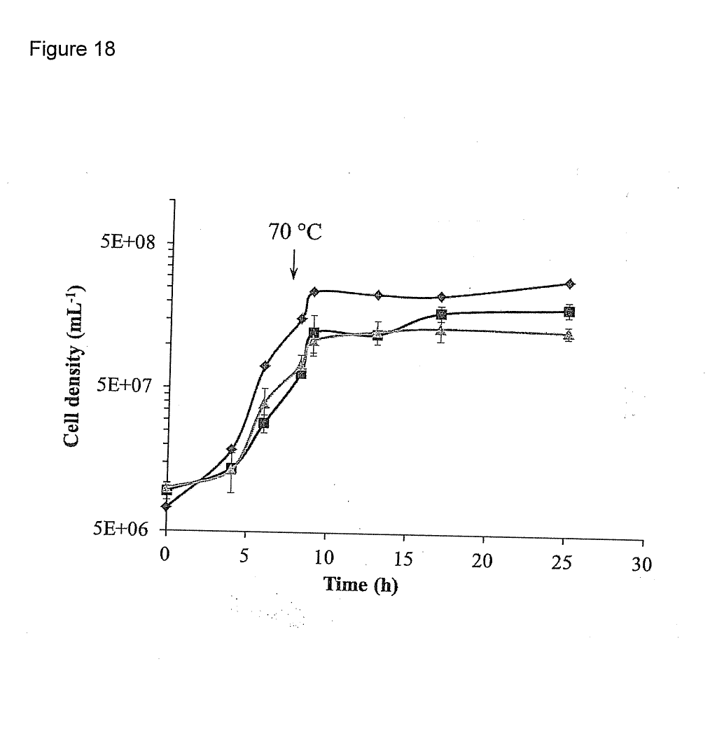

The five genes encoding the three enzymes (E1αβγ, E2, E3) of M. sedula SP1 were combined into a single synthetic operon with transcription driven by Pₛₗₚ, the native, constitutive promoter of the highly expressed S-layer protein (PF1399) (Chandrayan et al., J Biol Chem 287:3257-3264 (2012)). The M. sedula ribosomal binding sites (RBS) for E1(γ), E2 and E3 were replaced with RBSs for known highly-expressed P. furiosus proteins (FIG. 11A). The M. sedula RBS for E1β was retained since the two genes, E1□ and E1β, appear to be translationally-coupled. The SP1 operon was inserted into P. furiosus (strain COM1) at two genome locations. In P. furiosus strain PF506 the SP1 operon was inserted at the site of the pdaD marker (PF1623; FIG. 12), while the MW56 strain contained the SP1 operon between convergently-transcribed genes PF0574 and PF0575 (FIGS. 13 and 18) within a ˜100-bp region having little to no transcriptional activity according to a previous tiling array study of P. furiosus (Yoon et al., Genome Res 21:1892-1904 (2011)). The P. furiosus strains used here are summarized in Table 4.

TABLE 4
Strains used and constructed in this study.

| Strain | Parent | Genotype/Description | Reference |
|---|---|---|---|
| COM1 | DSM 3638 | ΔpyrF | 1 |
| ΔpdaD | COM1 | ΔpyrF | 2 |
| | | ΔpdaD::P_{gdh}pyrF | |
| PF506 | ΔpdaD | ΔpyrF | This work |
| | | ΔpdaD::pdaD Pₛₗₚ- | |
| | | E1αβγ-E2-E3 | |
| MW56 | COM1 | ΔpyrF P_{gdh}pyrF | This work |
| | | Pₛₗₚ-E1αβγ-E2-E3 | |
| MW43 | COM1 | ΔpyrF P_{gdh}pyrF | Example 4 |
| | | Pₛₗₚ-E7-E8-E9 | |

1: Lipscomb G L, et al. Appl Environ Microbiol. 2011. 77(7): 2232-8.
2: Hopkins R C, et al. PLoS One. 2011; 6(10): e26569

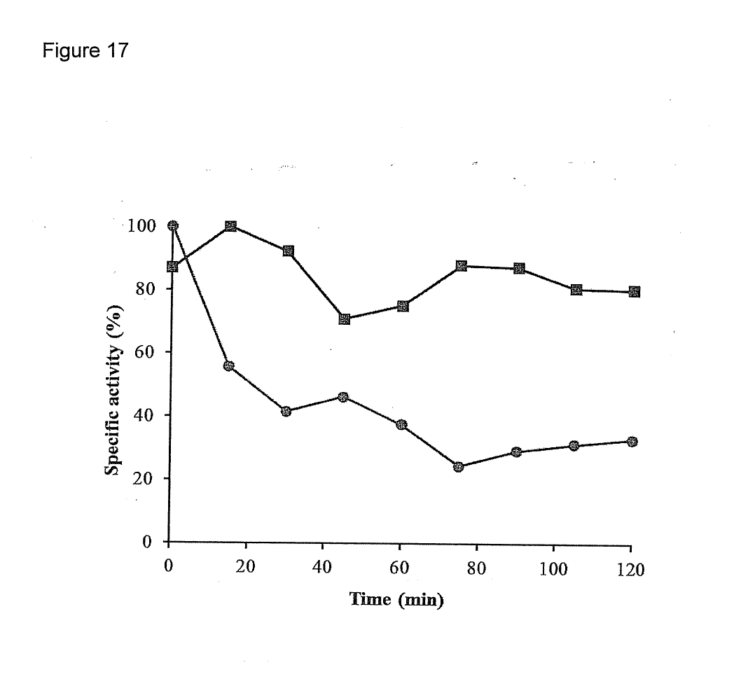

The premise for the temperature-dependent strategy is that P. furiosus (Topt 100° C.) shows little growth and has very low metabolic activity (Weinberg et al., J Bacteriol 187:336-348 (2005)) near the temperature at which the enzymes from M. sedula (Tₒₚₜ 73° C.) are expected to be optimally active. In the recombinant P. furiosus strains, the SP1 operon was under the control of a constitutive promoter (Pₛₗₚ), hence the operon may be transcribed at both 100° C. and 75° C. However, the resulting E1-E3 enzymes should be stable and active only near 75° C. P. furiosus strain PF506 and MW56 were, therefore, grown at 98° C. (to ˜1×10⁸ cells/ml) and then transferred to 75° C. (FIG. 15A). There was no measurable activity of E1, E2 or E3 in cell-free extracts prior to the temperature change, but all three activities were present in cells after 16 hr at 75° C. Moreover, the specific activities were comparable to those measured in extracts of M. sedula cells grown autotrophically on H₂ and CO₂ and to values reported by others (FIG. 15B) (Berg et al., Nat Rev Microbiol 8:447-460 (2010), Ramos et al., J Bacteriol 193:1201-1211 (2011)). When the recombinant P. furiosus strain PF506 was grown at 95° C. and then incubated for 16 hours at temperatures between 55° and 95° C., the maximum specific activity of the linked E2+E3 enzymes was measured in cultures incubated at 70 and 75° C., with dramatically lower values at 65 and 80° C. (FIG. 15C). This clearly indicates the ability of the M. sedula enzymes to fold correctly in P. furiosus optimally at 70-75° C., especially since significant E2+E3 activity in cell-free extracts can be measured at assay temperatures above 75° C. (FIG. 15D). Moreover, the enzymes are very thermostable, with a half-life of approximately 30 min at 90° C. (FIG. 17).

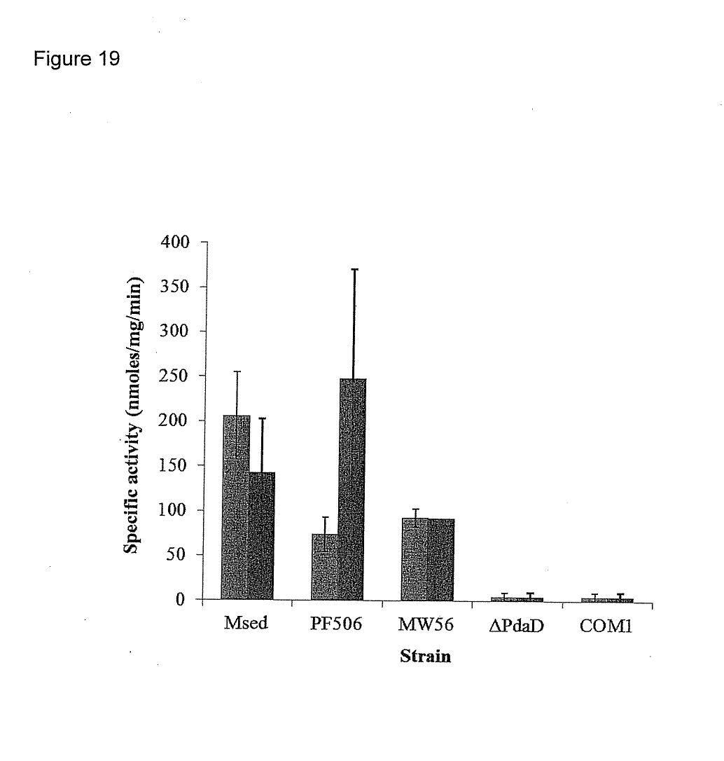

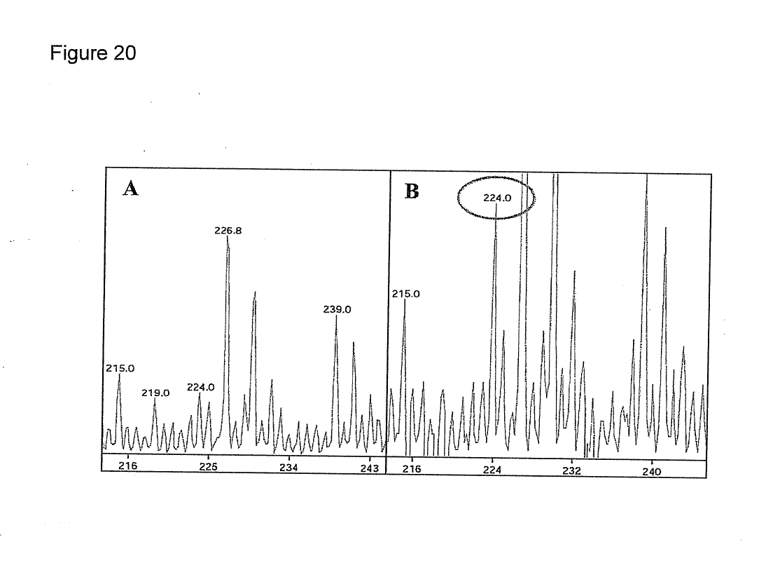

To determine the nature of the products of the SP1 pathway, recombinant P. furious strains PF506 and MW56 were grown at 95° C. (to ˜1×10⁸ cells/ml) and then transferred to 70° C. for 16 hours (FIG. 18). In extracts of these cells, the specific activities of the E1, E2, and E3 enzymes were comparable to those measured in extracts of autotrophically-grown M. sedula cells (FIG. 19). Two methods were used to detect 3-HP and to confirm its production by the SP1 pathway in the recombinant P. furiosus strains. In the presence of acetyl-CoA, NaHCO₃, and either NADPH or hydrogen gas as the electron donor, the 2-nitrophenylhydrazide-derivative (3-HP/HZ; m/z 224) was identified by electrospray ionization mass spectrometry (ESI-MS) in cell-free extracts of PF506 that was not present in extracts of the parent P. furiosus strain (FIG. 20). This was confirmed by gas chromatography-mass spectrometry (GC-MS) of the O-trimethylsilylate derivative of 3-HP (3HP/TMS) using malonyl-CoA and either NADPH or hydrogen gas as the electron donor (Table 5). The GC-MS also allowed quantitation of 3-HP/TMS and showed that approximately 150 μM 3-HP was produced from malonyl-CoA after a 2 hr incubation at 72° C. with extracts of PF506 containing NADP under hydrogen gas (FIG. 15, Table 5).

TABLE 5
GC-MS identification and quantitation of 3-HP produced from malonyl-
CoA and NADPH or H2 by cell-free extracts of P. furiosus strain
PF506. The assays were carried out in a total volume of 1 mL containing 0.25
mg of cell-free extract under H₂ in a shaking water bath. The amount
of 3-HP produced was determined after 2 hr at 72° C.

| | Added | | | | |
|---|---|---|---|---|---|
| | Electron | | Theoretical | 3-HP/Inositol | Estimated |
| Vial | Donor | Substrate | 3-HP | peak area | 3-HP |
| 1 | 2 mM | 2 mM | 1 mM | 0.0288 | 0.2 mM |
| | NADPH | malonyl-CoA | | | |
| 2 | 2 mM | 2 mM | 2 mM | 0.0467 | 0.3 mM |
| | NADPH, H₂ | malonyl-CoA | | | |
| 3 | 1 mM | 2 mM | 2 mM | 0.0274 | 0.2 mM |
| | NADP, H₂ | malonyl-CoA | | | |
| 4 | 1 mM | None | 0 | 0.0064 | 0.05 mM |
| | NADP, H₂ | (control) | | | |
| 5 | 1 mM | None | 2 mM | 0.2839 | 2.0 mM |
| | NADP, H₂ | (control) | | | |

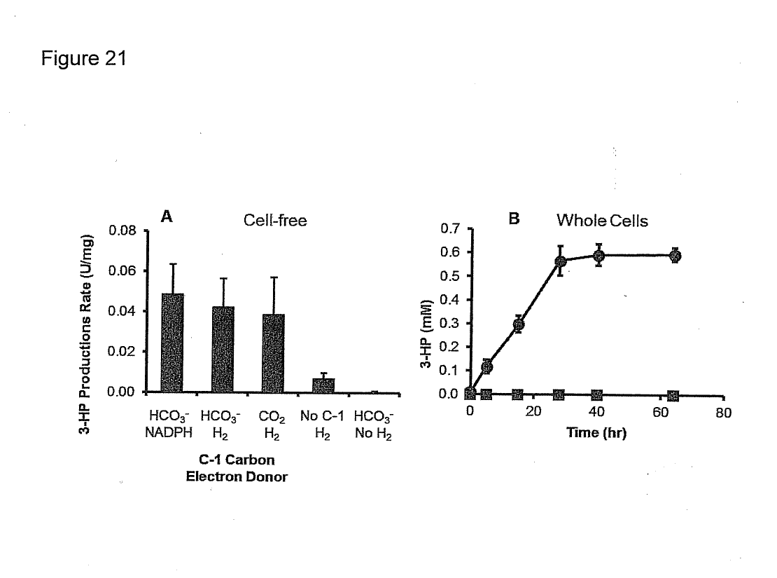

For routine analysis of 3-HP, a method was developed to extract 3-HP/HZ and to separate and quantitate it by HPLC. As shown in FIG. 21A, this method was used to confirm 3-HP production from acetyl-CoA and carbon dioxide by the combined action of the enzymes E1, E2, and E3 in cell-free extracts. As expected, P. furiosus did not appear to further metabolize 3-HP as the compound was stable when added to P. furiosus cultures. Moreover, the production of 3-HP from acetyl-CoA was dependent upon either NaHCO₃ or CO₂ as the C-1 carbon source and either NADPH or hydrogen gas (and NADP) as the electron donor (FIG. 21A). The incorporation of electrons from hydrogen gas and the carbon from CO₂ into a single desired product is essentially the paradigm for electrofuels (Hawkins et al., ACS Catalysis 1:1043-1050 (2011)).

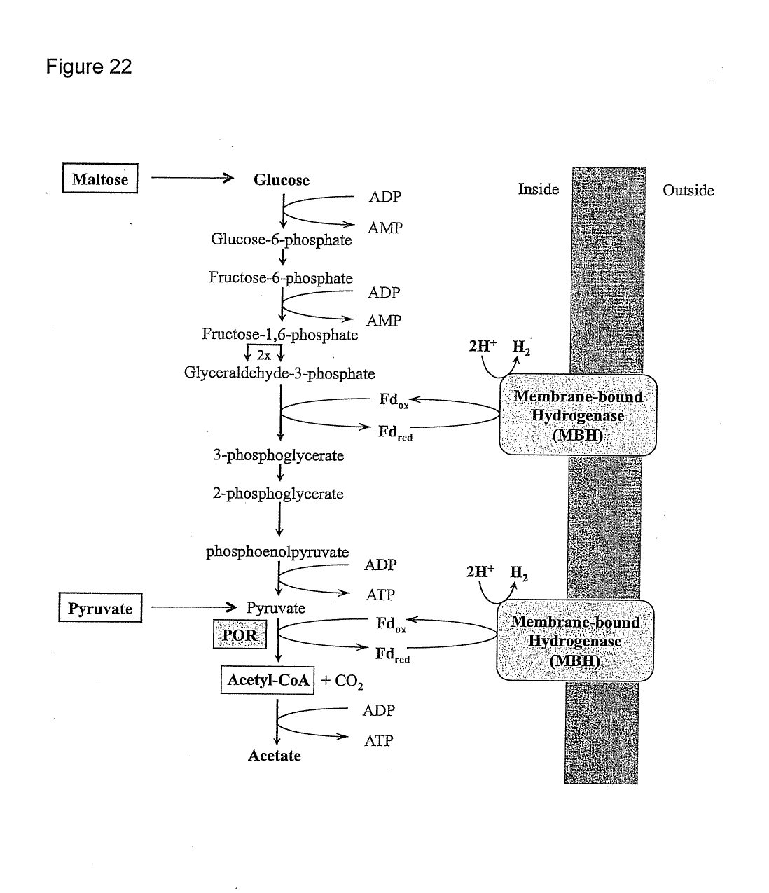

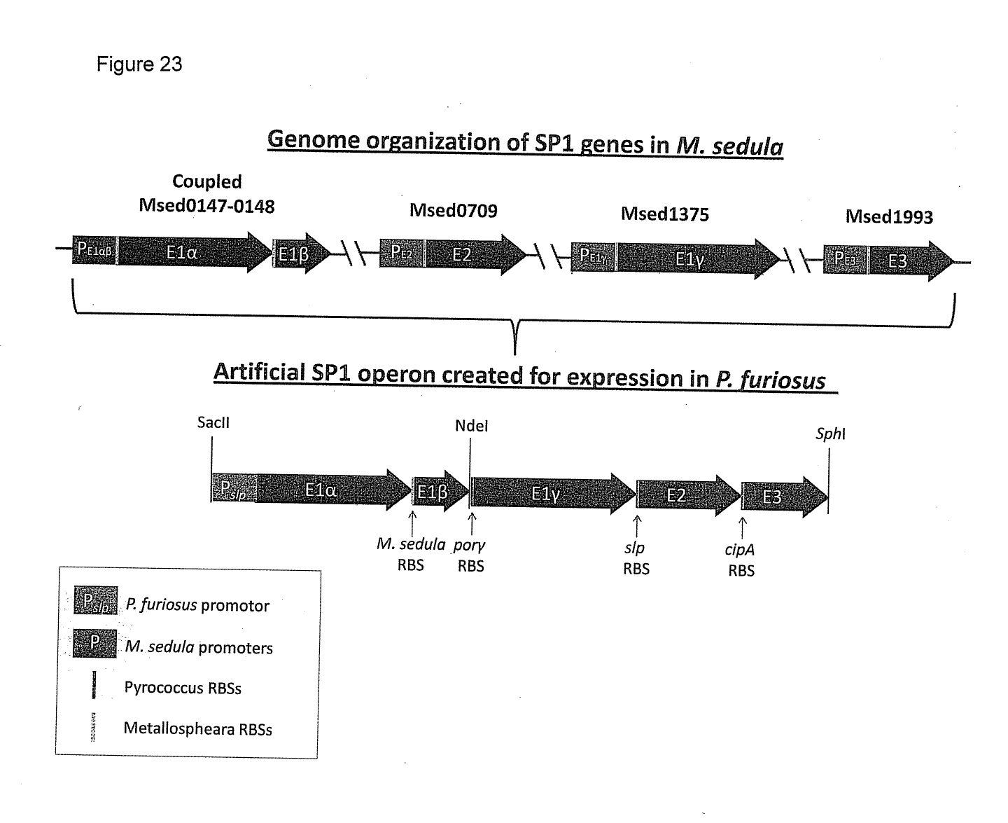

P. furiosus grows by fermenting sugars (such as the disaccharide maltose) to acetate, carbon dioxide and hydrogen and can also utilize pyruvate as a carbon source (Fiala et al., Arch. Microbiol. 145 (1986)). Acetyl-CoA and CO₂ are generated as the product of the pyruvate ferredoxin oxidoreductase (POR) reaction (FIG. 22). The reduced ferredoxin is oxidized by a membrane-bound hydrogenase to generate hydrogen gas (Sapra et al., Proc Natl Acad Sci USA 100:7545-7550 (2003)). Although the organism grows poorly at 75° C. (Weinberg et al., J Bacteriol 187:336-348 (2005)), it was expected that when whole cells were incubated at 75° C. with maltose or pyruvate, sufficient acetyl-CoA would be produced by the low metabolic activity of P. furiosus for the SP1 enzymes to produce 3-HP. Indeed, this proved to be the case. High cell density suspensions (≧10¹⁰ cells/ml) of P. furiosus strains PF506 and MW56 produced up to 0.2 mM 3-HP after one hour incubation at 75° C. in the presence of maltose, hydrogen gas, and NaHCO₃; and 3-HP production was dependent upon the presence of maltose or pyruvate (Table 6). Moreover, cultures of the recombinant P. furiosus strains, grown to late-log phase (˜1×10⁸ cells/ml.) at 98° C. on maltose, produced up to 0.6 mM 3-HP (60 mg/l) when subsequently incubated at 72° C. for up to 40 hours (FIG. 23B). A total of 135 μM of 3-HP was produced by a cell suspension of MW56 (5×10¹⁰ cells/mL) after 60 min at 75° C., and a total of 199 μM of 3-HP was produced by a cell suspension of PF506 (5×1010 cells/mL) after 60 min at 75° C.

TABLE 6
3-HP production using maltose or pyruvate as the source
of acetyl-CoA by whole cells of P. furiosus strains
PF506 and MW56. The amount of 3-HP indicated was present
in 1 mL of the cell suspension of P. furiosus.

| | |
|---|---|
| MW56 | PF506 |

| Pyruvate | Maltose | Pyruvate | Maltose |
|---|---|---|---|
| 155 nmol | 100 nmol | 70 nmol | 145 nmol |

In summary, this work demonstrates the principle of using hydrogen as the electron donor for carbon dioxide fixation into a product of great utility in the chemical industry, 3-HP. Moreover, it is carried out by an engineered heterotrophic hyperthermophile some 30° C. below the optimal growth temperature of the organism, conditions that support minimal growth, but sufficient metabolic activity is retained to sustain the production of 3-HP (Hawkins et al., ACS Catalysis 1:1043-1050 (2011)). The reaction can be accomplished by cell-free extracts, and also by whole cells in culture using sugar (maltose) as the source of the acetyl-CoA and ATP in a hydrogen- and CO₂-dependent manner. The feasibility of using hydrogen gas as the source of reducing power (NADPH) for chemical synthesis, in this case 3-HP, is also of high significance given the availability of inexpensive natural gas as a hydrogen source (Kreysa, G. ChemSusChem 2:49-55 (2009)). It is also important to note that in P. furiosus the low metabolic activity at 72° C. was sufficient to provide the ATP needed for carbon dioxide fixation. These results also bode well for the overall goal of incorporating into P. furiosus the complete M. sedula 3-HP/4-HB pathway in which two molecules of carbon dioxide are reduced to acetyl-CoA, which can then be converted to a variety of valuable products including biofuels (Hawkins et al., ACS Catalysis 1:1043-1050 (2011)). Clearly, there will be a balance between using a fixed carbon source (sugar) via the low metabolic activity of the host to produce ATP and the high catalytic activity of the heterologous enzymes to generate the desired product. The hydrogen-dependent fixation of carbon dioxide has enormous potential for the production of a variety of chemicals and fuels through strategic use of established biosynthetic pathways and exploiting the hyperthermophilicity of particular metabolically-engineered microbial hosts (Steen et al., Nature 463:559-562 (2010), Peralta-Yahya et al., Biotechnol J 5:147-162 (2010), Connor et al., Curr Opin Biotechnol 20:307-315 (2009), Kreysa, G. ChemSusChem 2:49-55 (2009)).

### Methods

NADPH-Dependent Assays for the E2, E2+E3 and E1+E2+E3 Reactions of SP1 and Phosphate-Dependent Assay for E1

All reactions were carried out in sealed anaerobic cuvettes at 75° C. containing 100 mM MOPS pH 7.5 (measured at room temperature), 5 mM MgCl₂, 5 mM DTT and the cell-free extract of P. furiosus (0.25 mg/mL). After addition of NADPH (to A340˜1.0), the relevant CoA derivative and other substrates (see below), NADPH oxidation was determined by decrease in the absorbance at 340 nm and rates were calculated based on the difference before and after the addition of the CoA thioester substrate. For the E2 assay, the additional substrate was 1 mM succinyl-CoA. For the E2+E3 assay, the additional substrate was 1 mM malonyl-CoA. For the E1+E2+E3 assay, the additional substrates were 1 mM acetyl-CoA, 1 mM ATP, and 10 mM NaHCO3. The product of E1 activity, malonyl-CoA, is used by E2 and the product of that reaction, malonate semialdehyde, is used as a substrate for E3, both in NADPH-dependent reactions. For the E1 assay, which measured phosphate release, the cell-free extract was added to 0.1 mg/mL to 100 mM MOPS pH 7.5 (at room temperature), 5 mM MgCl₂, and 5 mM DTT. Added substrates were 10 mM NaHCO₃, 1 mM ATP, and 1 mM acetyl-CoA. The sealed anaerobic vials were incubated at 75° C. and 20 μL samples were taken out at 0, 2, and 4 min and added to a 96 well plate. The samples were diluted with 180 μL of water before the addition of 30 μL of BioVision (Mountain View, Calif.) phosphate assay reagent. The absorbance at 650 nm was measured and the amount of phosphate produced was calculated using a molar extinction coefficient of 90,000 M⁻¹cm⁻¹.

Construction of a Synthetic SP1 Operon for Expression of Genes Encoding the E1, E2 and E3 Enyzmes of the M. sedula 3HP/4HB Cycle

PCR was performed using P. furiosus or M. sedula genomic DNA to generate the individual PCR products of the P. furiosus S-layer promotor (Pslp) and the five M. sedula SP1 genes, consisting of coupled E1αβ (Msed_{—}0147-Msed_{—}0148), E1γ (Msed_{—}1375), E2 (Msed_{—}0709) and E3 (Msed_{—}1993). P. furiosus ribosomal binding sites, consisting of 11-14 bp of sequence upstream of highly-expressed proteins, were added in front of E1γ (5′-GGAGGTTTGAAG (SEQ ID NO:42), sequence upstream from pory, PF0791), E2 (5′-GGGAGGTGGAGCAT (SEQ ID NO:43), sequence upstream from slp, PF1399), and E3 (5′-GGTGATATGCA (SEQ ID NO:87), sequence upstream from cipA, PF0190). The primer sequences are given in Table 7. SOE-PCR (splicing by overlap extension and PCR, (Horton et al., Gene 77:61-68 (1989)) was performed to combine the individual PCR products and generate the expression cassette for SP1 (see FIG. 11A).

TABLE 7
Primers used in the construction of the synthetic SP1 operon.

| Primer target | Direction, | 5′ to 3′ sequence |
|---|---|---|
| Pslp | Forward | GAATCCCCGCGGCCCGGGCTGGCAGAATAGAA (SEQ ID NO: 44) |
| | Reverse | GCAACCAAAACTCTACTAAAGGGTGGCATTTTTCTCCACCTCCCAATAATCTG |
| | | (SEQ ID NO: 45) |
| Msed_0147-0148 | Forward | ATGCCACCCTTTAGTAGAGTTTTGG (SEQ ID NO: 46) |
| | Reverse | GTTGCAGTCATCTTCAAACCTCCTTACTTTATCACCACTAGGATATCTCC |
| | | (SEQ ID NO: 47) |
| Msed1375 | Forward | GTGATAAAGTAAGGAGGTTTGAAGATGACTGCAACTTTTGAAAAACCGGAT |
| | | (SEQ ID NO: 48) |
| | Reverse | CGTTCTCCTCATATGCTCCACCTCCCTTAGAGGGGTATATTTCCATGCTTC |
| | | (SEQ ID NO: 49) |
| Msed_0709 | Forward | GGCAATGTCATATGAGGAGAACGCTAAAGGCCGCAATTC (SEQ ID NO: 50) |
| | Reverse | CCTTTTCAGTCATTGCATATCACCTCATCTCTTGTCTATGTAGCCCTTC, |
| | | (SEQ ID NO: 51) |
| Msed_1993 | Forward | TAGACAAGAGATGAGGTGATATGCAATGACTGAAAAGGTATCTGTAGTTGGAG |
| | | (SEQ ID NO: 52) |
| | Reverse | CCAATGCATGCTTATTTTTCCCAAACTAGTTTGTATACCTTC (SEQ ID NO: 53) |

Construction of Vectors for Insertion of the SP1 Operon into P. furiosus Strains ΔpdaD and COM1

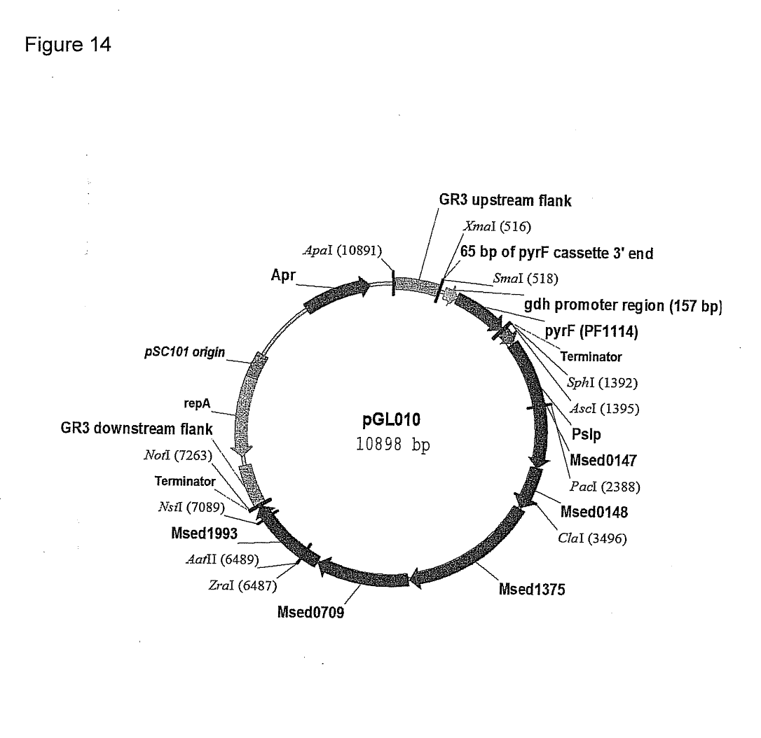

The SP1 expression cassette (FIG. 11B) was cloned into pSPF300 15, generating the plasmid pALM506-4, to be used for targeted insertion of the synthetic SP1 operon into the P. furiosus ΔpdaD strain (FIG. 12). SOE-PCR (Horton et al., Gene 77:61-68 (1989)) was used to combine ˜0.5 kb flanking regions targeting homologous recombination in the integenic space between convergent genes PF0574-PF0575, with a marker cassette, including restriction sites for cloning. The marker cassette for uracil prototrophic selection consisted of the pyrF gene driven the gdh promoter region (Pgdh, 157 bases of DNA sequence immediately upstream from the translation start of the glutamate dehydrogenase gene, PF1602) and terminated with the terminator sequence consisting of 12 bases of the 3′ UTR of the hpyA1 gene (5′-aatcttattag (SEQ ID NO:54), PF1722). A 65-b sequence of the 3′ end of the marker cassette (5′-ctaaaaaagattttatcttgagctccattctttcacctcctcgaaaatcttcttagcggcttccc (SEQ ID NO:55)) was repeated at the beginning of the cassette to serve as a homologous recombination region for selection of marker removal from the transformed strain that would allow for iterative use of the marker in the same strain (Shen et al., Appl Environ Microbiol 77:2905-2915 (2011)). Vector pGL007 targeting homologous recombination at the PF0574-PF0575 intergenic space was constructed by cloning the SOE-PCR product into pJHW006 (Lipscomb et al., Appl Environ Microb 77:2232-2238 (2011)) (FIG. 13). The SP1 expression cassette was PCR-amplified from pALM506. A terminator sequence was added to the 3′ end of the operon (5′-aatcttattag (SEQ ID NO:54), from the 3′ UTR of PF1722), and the construct was cloned into the AscI-NotI sites of pGL007 to make pGL010 (FIG. 14), for targeted insertion of the SP1 operon at the PF0574-PF0575 intergenic space.

Transformation of P. furiosus ΔpdaD Strain to Yield P. furiosus Strain PF506 Containing the SP1 Operon

Transformation of P. furiosus ΔpdaD strain was performed as previously described for COM1 (Lipscomb et al., Appl Environ Microb 77:2232-2238 (2011)) except that the defined medium contained maltose instead of cellobiose as the carbon source and was supplemented with 0.1% w/v casein hydrolysate. Briefly, pALM506-1 was mixed (at ˜5 μg plasmid DNA per mL culture) with an aliquot of a fresh overnight culture of ΔpdaD grown in defined maltose (DM) medium containing 0.1% w/v casein hydrolysate and 4 mM agmatine. The transformation mixtures were spread on DM plate medium containing 0.1% w/v casein hydrolysate and 20 μM uracil and incubated at 90° C. for ˜95 h. Transformant colonies were further purified by six serial transfers in DM liquid medium containing 0.1% w/v casein hydrolysate and 20 μM uracil. The presence of the insert in the transformed strains was verified by PCR screening of isolated genomic DNA.

Transformation of P. furiosus COM1 Strain to Yield P. furiosus Strain MW56 Containing the SP1 Operon

Transformation of COM1 was performed as previously described (Peralta-Yahya et al., Biotechnol J 5:147-162 (2010)), except that linear plasmid DNA was used for transformation. Briefly, pGL010 was linearized by restriction digest and mixed (at a final concentration of ˜2 μg/mL DNA) with an aliquot of a freshly grown culture of COM1, grown in defined cellobiose medium plus 20 μM uracil. Transformation mixtures were spread on defined cellobiose plate medium without uracil and incubated at 95° C. for ˜60 hr. Transformant colonies were further purified on defined cellobiose plate medium without uracil twice. Strains were verified by PCR screening of isolated genomic DNA and sequencing of PCR products amplified from the target regions.

Growth of P. furiosus for Biochemical Assays and Product Analysis

P. furiosus strains were cultured as previously described (Peralta-Yahya et al., Biotechnol J 5:147-162 (2010)) in a sea-water based medium containing 5 g/L maltose and 5 g/L yeast extract, 0.5 μg/L riboflavin, and 20 μM uracil or 4 mM agmatine as needed. The media were made anaerobic by the addition of 0.5 g/L cysteine HCl, 0.5 g/L Na2S (dissolved in 50 mL water), followed by 1.0 g/L NaHCO3 and 1 mM potassium phosphate buffer (from a 1 M stock at pH 6.8). If needed, the pH of the medium was adjusted to 6.8 with HCl before degasing. Cultures were inoculated to 1×10⁷ cells/mL and incubated at 95° C. until cell densities reached ˜1×10⁸ cells/mL. Cultures were then cooled at room temperature until the temperature reached 70 to 75° C. when they were placed in an incubator set to a temperature in the range of 70 to 75° C. for up to 48 hours. Cell densities were calculated from counting a sample in a Hausser counting chamber. To obtain cell-free extracts, P. furiosus cell pellets were suspended in 100 mM MOPS, pH 7.5 (3 mL buffer/g cells), containing DNase I (0.5 μg/mL) in an anaerobic chamber. The slurry was stirred for 30 minutes, lysing the cells by osmotic shock. The cell extract was then centrifuged at 100,000×g for 1 hr. The resulting cell-free extract was diluted with 100 mM MOPS, pH 7.5, and re-concentrated three-times with a 3 kDa centrifugation filter, sealed in a vial to maintain anaerobic conditions and stored at −80° C.

Growth of M. sedula for biochemical assays and product analysis. M. sedula (DSM 5348) was grown autotrophically at 70° C. with micro-bubblers feeding 1 mL/min 80/20 H2/CO₂ and 100 mL/min air in a defined medium, DSMZ 88 pH 2.0, containing: 1.30 g/L (NH₄)2SO₄, 0.28 g/L KH₂PO₄, 0.25 g/L MgSO₄.7H₂O, 0.07 g/L CaCl₂.2H₂0, 0.02 g/L FeCl₃.6H₂0, 1.80 mg/L MnCl₂.4H₂0, 4.50 mg/L Na₂B4O₇.10H₂O, 0.22 mg/L ZnSO₄.7H₂O, 0.05 mg/L CuCl₂.2H₂O, 0.03 mg/L Na₂MoO₄.2H₂O, 0.03 mg/L VOSO₄.2H₂O, and 0.01 mg/L CoSO₄. To obtain cell-free extracts, M. sedula frozen cell pellets were anaerobically suspended in 50 mM Tris HCl pH 8.0 containing 0.5 μg/mL DNase 1 (2 mL buffer/g cell paste) and stirred for 1 hr in an anaerobic chamber. M. sedula undergoes osmotic lysis when placed in the hypotonic lysis buffer, and the DNA released is digested by DNAse I. The cell extract was then centrifuged at 100,000×g for 1 hr. The resulting cell-free extract was sealed in a vial to maintain anaerobic conditions and stored at −80° C.

Source of 3-Hydroxypropionic Acid (3-HP)

The 3-HP used as a standard for detection and quantitation was product number H0297 (30%, w/v, in water) obtained from TCI America (http://www.tciamerica.net/). Using HPLC and ¹H NMR, the purity was estimated at approximately 75% with the remaining 25% as an ether-linked dimeric form (3,3′-oxydipropanoic acid).

GC-MS Detection of 3-HP

A sample of the enzyme assay mixture was spiked with 20 μg of inositol as an internal standard. For hydrolysis of proteins, the samples were freeze-dried, then incubated in 2 M TFA at 80° C. for 1 hr then dried under nitrogen. The samples were then per-O-trimethylsilylated by treatment with Tri-Sil (Pierce) at 80° C. for 30 minutes. GC-MS analysis of the TMS derivatives was performed on an AT 7890n GC interfaced to a 5975C MSD, using a Grace EC-1 column (30 m×0.25 mm). The exact mass of 3-HP-TMS is 162.

2-Nitrophenyl Hydrazine Derivatization of 3HP

The steps to derivatize 3HP were modified from those previously reported 29 briefly, a 100 μL sample of cell-free extract was added to 200 μL ethanol. Followed by addition of 200 μL 20 mM 2-nitrophenyl hydrazine in 100 mM HCl/ethanol (1:1). and 200 μL 250 mM 1-ethyl-3-(3-Dimethylanimopropyl)-N′-ethylcarbodiimide hydrochloride (1-EDC.HCL) in 3% pyridine in ethanol (v/v). Samples were heated at 60° C. for 20 mins, followed by addition of 100 μL of 15% (W/V) KOH. Samples were heated again at 60° C. for 15 minutes, and cooled before acidification with 50% HCl to pH between 4-6. Aliquots 10-50 μL were analyzed by HPLC as described above.

Ether Extraction of 3HP-Hydrazide

The 3HP-hydrazide was ether extracted as follows: 1 mL of 1 M KPO4 buffer pH 7.0 and 1.5 mL of ether were added to 800 μL of the cooled derivatized sample. The samples were then centrifuged for 10 min at 6,000×g to separate the phases. The top ether layer was removed and transferred to a new tube and the ether was evaporated. The dried sample was resuspended in 200 μL ethanol, and a 10-50 μL aliquots were run on the HPLC.

HPLC Detection of 3-HP-Hydrazide

The column and run conditions were as follows: column, Supelco LiChrosorb RP-8 (5 μm); solvent system, A 0.05% TFA, B 100% acetonitrile; gradient 0-20 min, 0-100% B, 20-22 min: 100% B; flow rate: 1 mL/min; temperature: 30° C.

ESI-MS Detection of 3-HP-Hydrazide

The derivatized 3HP samples were extracted with ether, dried, and re-constituted in methanol. The resulting samples were analyzed by direct injection on a Perkin-Elmer API 1 plus in negative mode. The exact mass of the anion 3-HP-hydrazide derivative is 224.

Production of 3-HP In Vitro from Malonyl-CoA by E2+E3 and from Acetyl-CoA by E1+E2+E3

To the P. furiosus extract (1-2 mg/mL) in buffer containing 100 mM MOPS pH 7.5, 5 mM MgCl₂, and 5 mM DTT, was added 1-2 mM malonyl-CoA (for E2+E3) or 10 mM NaHCO₃ (or 100% CO2 in the gas phase), 2 mM ATP and 2 mM acetyl-CoA (for E1+E2+E3). The electron source was 2 mM NADPH or 0.5 mM NADP+ with 20% H2 in the headspace. Sealed anaerobic vials containing the reaction mixture were incubated at 75° C. for up to 2 hrs. Samples for 3-HP analysis were derivatized with 2-nitrophenyl hydrazine and analyzed by HPLC as described above.

Product analysis of E1+E2+E3 activities in whole cells of P. furiosus. P. furiosus strains PF506 and MW56 were grown in 2 L cultures at 95° C. for 10 hours until cell densities reached 1×10⁸ cells/mL when they were cooled and incubated at 75° C. for 16 hours. Harvested cells were suspended to 5×10¹⁰ cells/mL in 100 mM MOPS pH 7.5 and base salts (28 g/L NaCl, 3.5 g/L MgSO₄.7H₂O, 2.7 g/L MgCl₂.6 H₂O, 0.33 g/L KCl, 0.25 g/L NH₄Cl, 0.14 g/L CaCl₂.2H₂0). The cell suspension was sealed in a serum vial, degassed with argon, and cysteine HCl was added to 0.5 g/L cysteine. Added substrates were 10 mM NaHCO₃ and either 10 mM maltose or 40 mM pyruvate. The vials were then degassed with H₂ and incubated at 75° C. for 60 minutes. Samples for 3-HP analysis were derivatized with 2-nitrophenyl hydrazine, using 1 mM p-hydroxyphenyl acetic acid as an internal standard, ether-extracted and analyzed by HPLC as described above.

Analysis of the P. furiosus Culture Medium for 3-HP

P. furiosus strains PF506, MW56 and COM1 were grown at 98° C. in 50 mL cultures with maltose as the carbon source until a cell density of 8×10⁷ cells/mL was reached and the incubation temperature was shifted to 72° C. for up to 4 days. Sample (1 mL) were periodically removed, centrifuged (10,000×g, 10 min) and to a 100 μl aliquot of the supernatant (the spent medium) 1 mM p-hydroxyphenyl acetic acid was added as an internal standard. The sample was derivatized with 2-nitrophenyl hydrazine, ether extracted and analyzed by HPLC as described above.

### Example 4

### Construction of P. Furiosus Strains PF506 and MW56 Containing the SP1 Pathway for 3-Hydroxypropionate Production and the Control Strain MW43 for Optimizing Production of M. sedula Enzymes in P. furiosus

The five genes encoding the three enzymes (E1αβγ, E2, E3) of the M. sedula 3HP/4-HB CO₂ fixation sub pathway I (SP1) are scattered across the M. sedula genome (FIG. 23). These genes have been combined into a single artificial operon using overlapping SOE-PCR (splicing by overlap extension and PCR, Horton, et al. 1989. Gene 77, 61), followed by integration of the expression cassette into the P. furiosus genome. Transcription of the artificial SP1 operon in P. furiosus is driven by Pₛᵢₚ, the native, constitutive promoter of the highly expressed S-layer protein (Chandrayan, S. K. et al. 2012. J. Biol. Chem. 287, 3257-3264). To optimize translation of the SP1 genes in P. furiosus, the native M. sedula ribosomal binding sites (RBSs) for E1γ, E2 and E3 were replaced with optimal P. furiosus RBSs/linker regions for predicted and known highly expressed proteins, while retaining the M. sedula RBS for E1β since the two genes, E1α and E1β, appear to be translationally coupled.

Strategy for operon expression (SP1 and SP2B) in P. furiosus. The SP1 operon was inserted into the COM1 strain of P. furiosus at two locations on the genome giving rise to two recombinant P. furiosus strains, PF506 and MW56. In addition, a control strain, MW43, was constructed to explore the temperature dependent expression of M. sedula genes in P. furiosus. MW43 contained subpathway 2B (SP2B; E7, E8 and E9) of the 3HP/4HB cycle.

PF506: the SP1 operon was inserted at the site of the pdaD marker.

MW56: the SP1 operon was inserted into one (GR3) of eleven genome regions previously identified as having little or no transcriptional activity.

MW43: the SP2B operon was inserted into GR2.

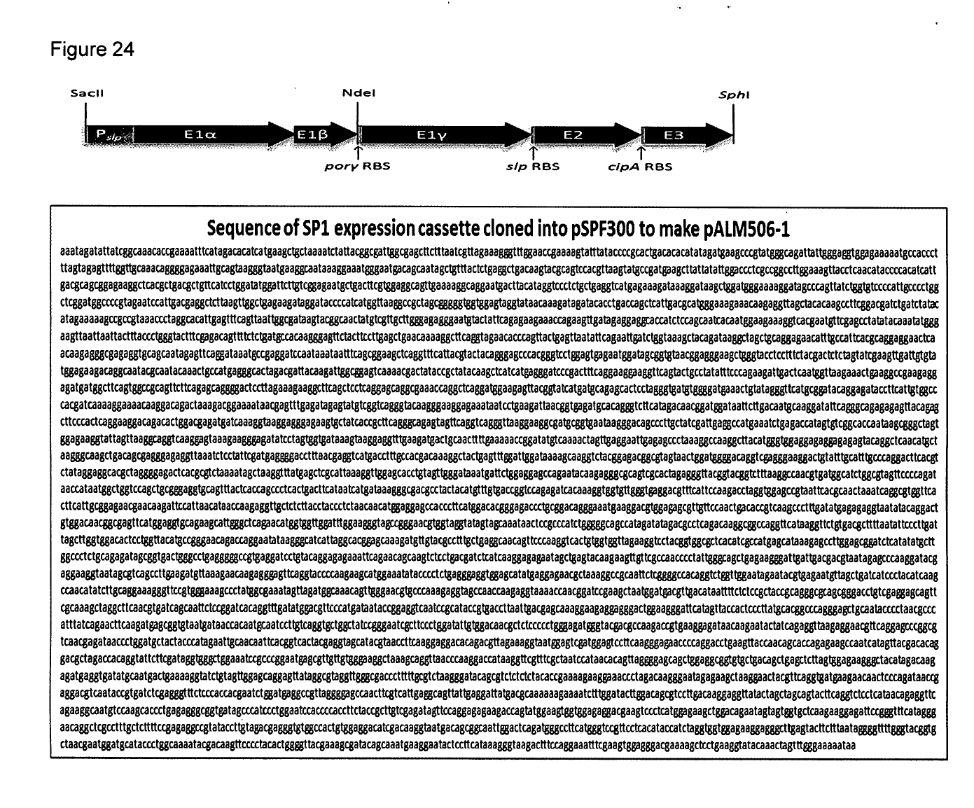

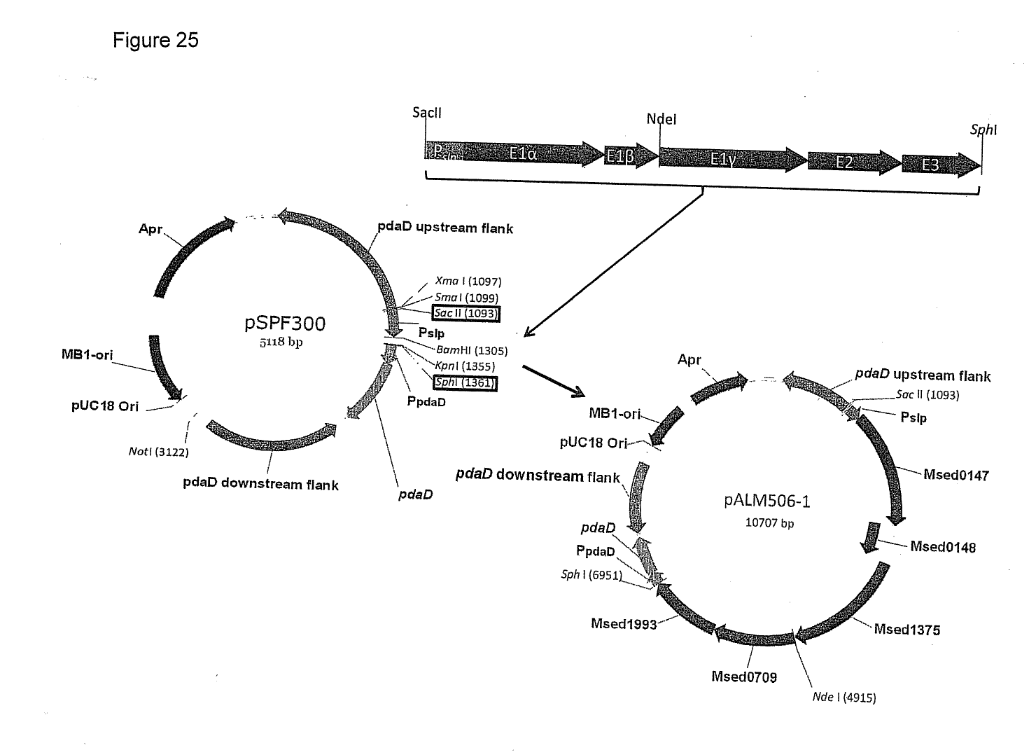

Construction of synthetic operon for expression of SP1 genes. PCR was performed using P. furiosus genomic DNA or M. sedula genomic DNA to generate the individual PCR products of the P. furiosus S-layer promotor and the five M. sedula SP1 genes, consisting of coupled E1αβ (Msed_{—}0147-Msed_{—}0148), E1γ (Msed_{—}1375), E2 (Msed_{—}0709) and E3 (Msed_{—}1993). PCR primers were designed to contain optimized P. furiosus ribosomal binding sites and spacing (Table 7) and to allow splicing of the individual PCR products generated (Table 7 and Table 8). SOE-PCR (Horton, et al. 1989. Gene 77, 61) was performed to combine the individual PCR products and generate the expression cassette for SP-1 (FIG. 24). The expression cassette was digested with SacII and SphI restriction enzymes and cloned into the SacII-SphI sites of the transformation vector, pSPF300 (Hopkins et al, 2011), generating the transformation plasmid, pALM506-1, for targeted insertion into the ΔpdaD strain of P. furiosus (FIG. 25).

TABLE 8
Upstream and intergenic regions with optimized native Pf RBS sequences and spacing.

| | | |
|---|---|---|
| E1-α: Msed_0147 | GGGAGGTGGAGAAAATG | PF1399 (slp, S-layer protein) RBS |
| | (SEQ ID NO: 81) | |
| E1-β: Msed_0148 | GGGTGATGTGGGGATGA | Msed0148 (native Msed RBS: coupled E1-αβ) |
| | (SEQ ID NO: 82) | |
| E1-γ: Msed_1375 | TAAGGAGGTTTGAAGATG | PF0791 (pory: Pyruvate ferredoxin |
| | (SEQ ID NO: 83) | oxidoreductase γ) RBS |
| E2: Msed_0709 | TAAGGGAGGTGGAGCATATG | PF1399 (slp, S-layer protein) RBS |
| | (SEQ ID NO: 84) | |
| E3: Msed_1993 | TGAGGTGATATGCAATG | PF0190 (cipA, cold induced protein A) RBS) |
| | (SEQ ID NO: 85) | |

Transformation of P. furiosus ΔpdaD strain to yield P. furiosus strain PF506 containing the SP1 operon. Transformation of P. furiosus ΔpdaD strain was performed as previously described for COM1 (Lipscomb, et al. 2011. Appl Environ Microbiol. 77(7):2232-8) with a few changes, in that sequence-verified plasmid DNA was used for transformation and the defined medium contained maltose instead of cellobiose as the carbon source and was supplemented with 0.1% w/v casein hydrolysate. Briefly, pALM506-1 was mixed (at ˜5 μg plasmid DNA/mL culture) with an aliquot of a fresh overnight culture of ΔpdaD grown in defined maltose (DM) medium containing 0.1% w/v casein hydrolysate and 4 mM agmatine. The transformation mixtures were spread on DM plate medium containing 0.1% w/v casein hydrolysate and 20 μM uracil and incubated at 90° C. for ˜95 h. Transformant colonies were further purified by six serial transfers in DM liquid medium containing 0.1% w/v casein hydrolysate and 20 μM uracil. The presence of the insert in the transformed strains was verified by PCR screening of isolated genomic DNA.

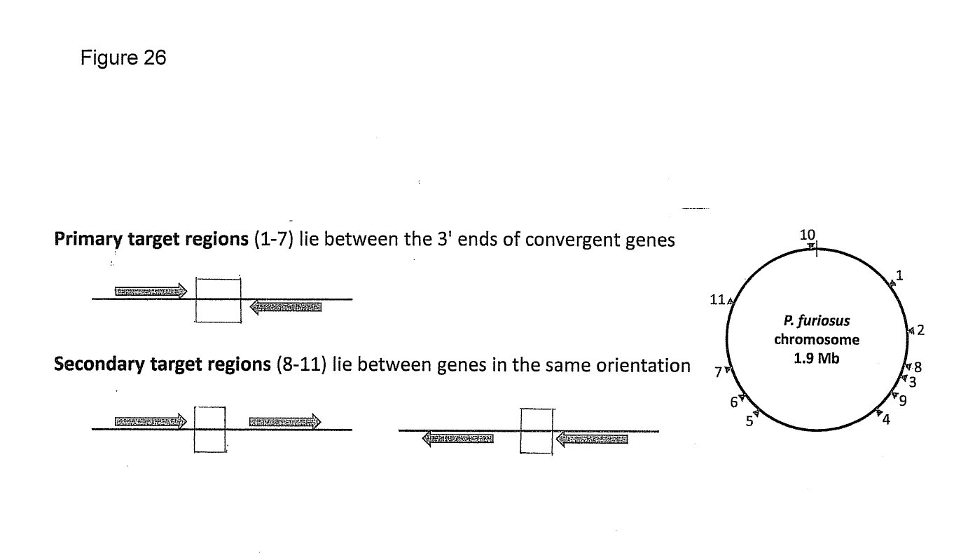

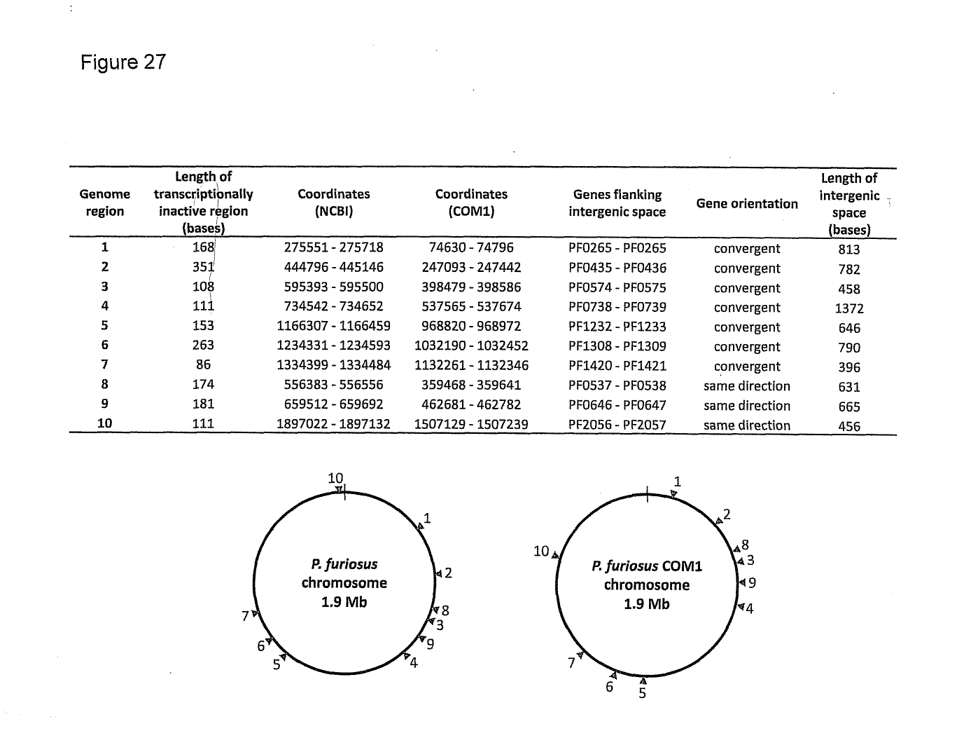

Determining transcriptionally inactive regions for foreign gene insertion. P. furiosus intergenic genome regions with little to no transcriptional activity were found using tiling array data of gene expression in wild-type P. furiosus from early log to early stationary phase, relative to a mid-log time point ((Yoon, et al. 2011. Genome Res. 21(11):1892-904), FIG. 26). Primary targets consisted of intergenic space between convergent genes, so as to avoid gene promoter regions. Secondary targets consisted of intergenic space between genes in the same orientation, separated by at least ˜450 bases. Ten total genome regions with little to no transcriptional activity were identified for use as foreign gene insertion sites. Tiling array data was mapped to the NCBI reference genome sequence (P. furiosus DSM3638); however, the genetically tractable strain of P. furiosus, COM1, has some genome rearrangements which affect the positions of the genome regions within the chromosome (Lipscomb G L, et al. 2011. Appl Environ Microbiol. 77(7):2232-8, Bridger S L, et al. 2012. J Bacteriol. 194(15):4097-106) (FIG. 27). Namely, genome region 10 was located within a region of the P. furiosus genome which was inverted in the COM1 strain.

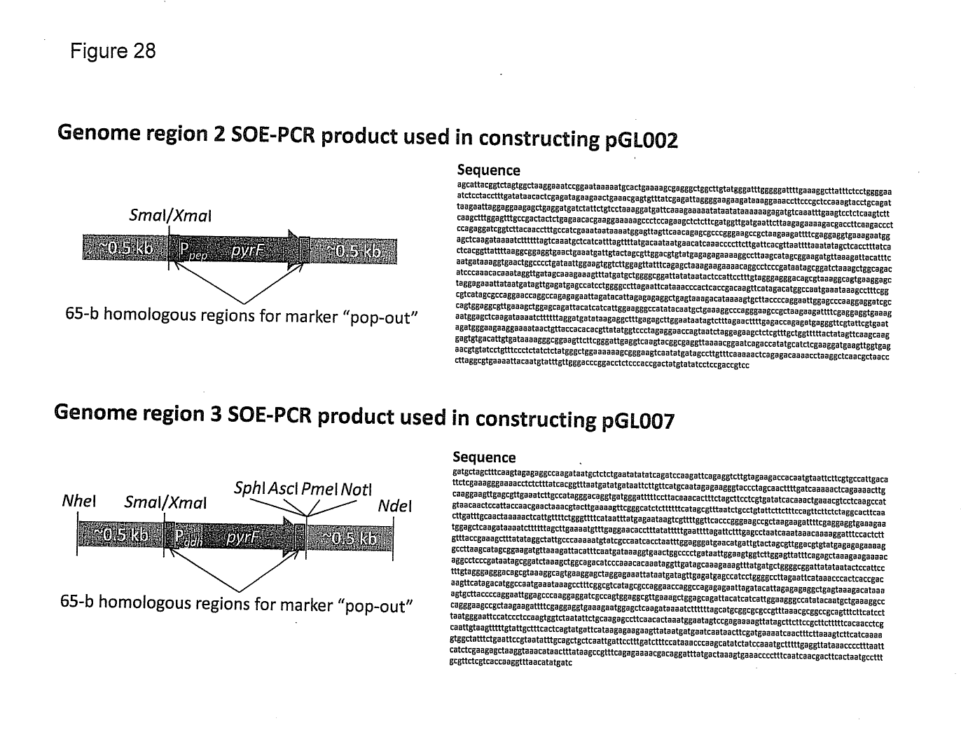

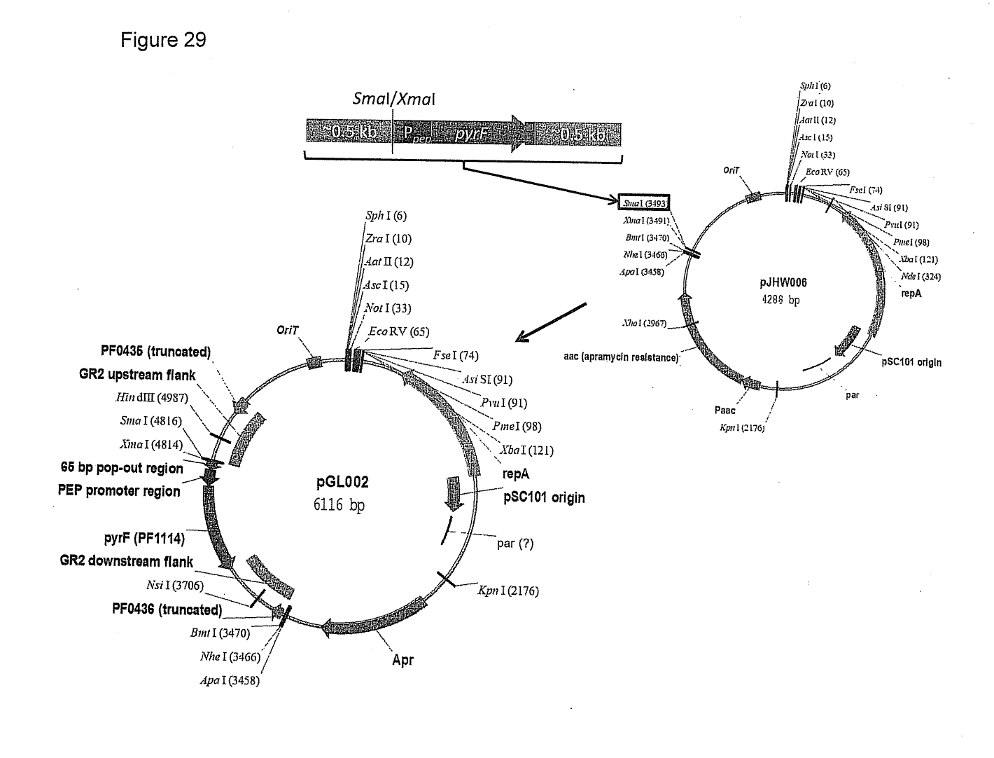

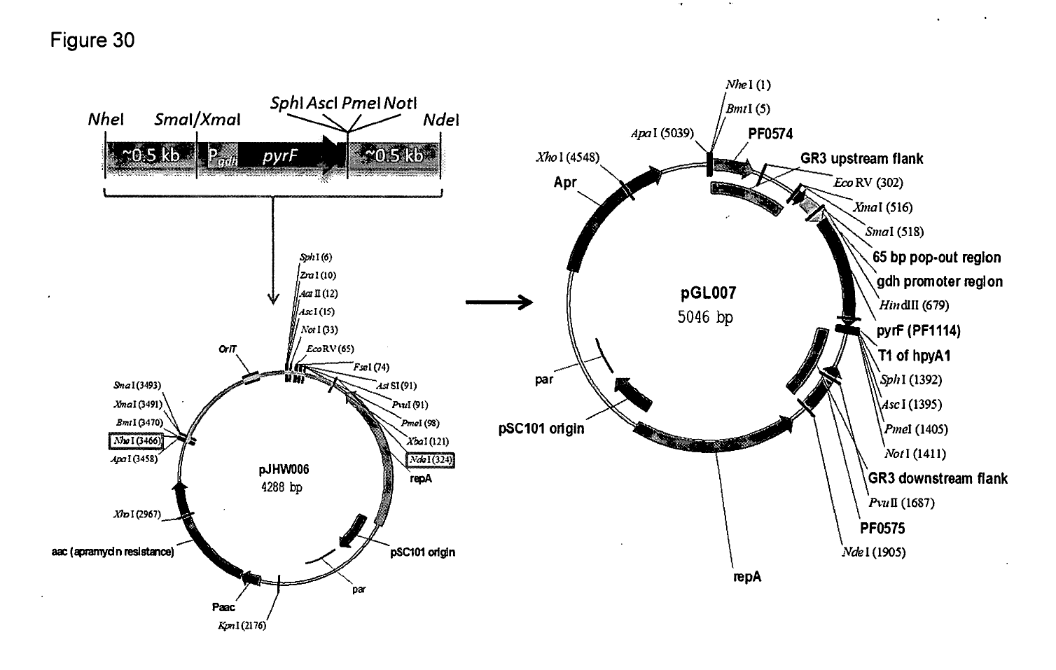

Construction of vectors targeting insertion at genome regions 2 and 3. SOE-PCR (splicing by overlap extension and PCR, Horton, et al. 1989) was used to combine ˜0.5 kb flanking regions targeting homologous recombination at genome region 3 (between convergent genes PF0574-PF0575, see FIG. 27), with a marker cassette, including restriction sites for cloning. The marker cassette for uracil prototrophic selection consisted of the pyrF gene driven by either the pep promoter region (Pₚₑₚ, 123 bases of DNA sequence immediately upstream from the translation start of the PEP synthase gene, PF0043) or the gdh promoter region (P_{gdh}, 157 bases of DNA sequence immediately upstream from the translation start of the glutamate dehydrogenase gene, PF1602) and terminated with the terminator sequence consisting of 12 bases of the 3′ UTR of the hpyA1 gene (5′-aatcttttttag (SEQ ID NO:54), PF1722). A 65-b sequence of the 3′ end of the marker cassette (5′-ctaaaaaagattttatcttgagctccattctttcacctcctcgaaaatcttcttagcggatccc (SEQ ID NO:55)) was repeated at the beginning of the cassette to serve as a homologous recombination region for selection of marker removal from the transformed strain which would allow for iterative use of the marker in the same strain (Farkas J, et al. Appl Environ Microbiol. 2012. 78(13):4669-76) (FIG. 28). Vector pGL002, targeting genome region 2, was constructed by cloning the SOE-PCR products into the SmaI site of pJHWOO6 (FIG. 29), and vector pGL007 targeting genome region 3 was constructed by cloning the SOE-PCR product into the NdeI-NheI sites of pJHW006 (FIG. 30) (Lipscomb, et al., Appl Environ Microb 77:2232-2238 (2011)).

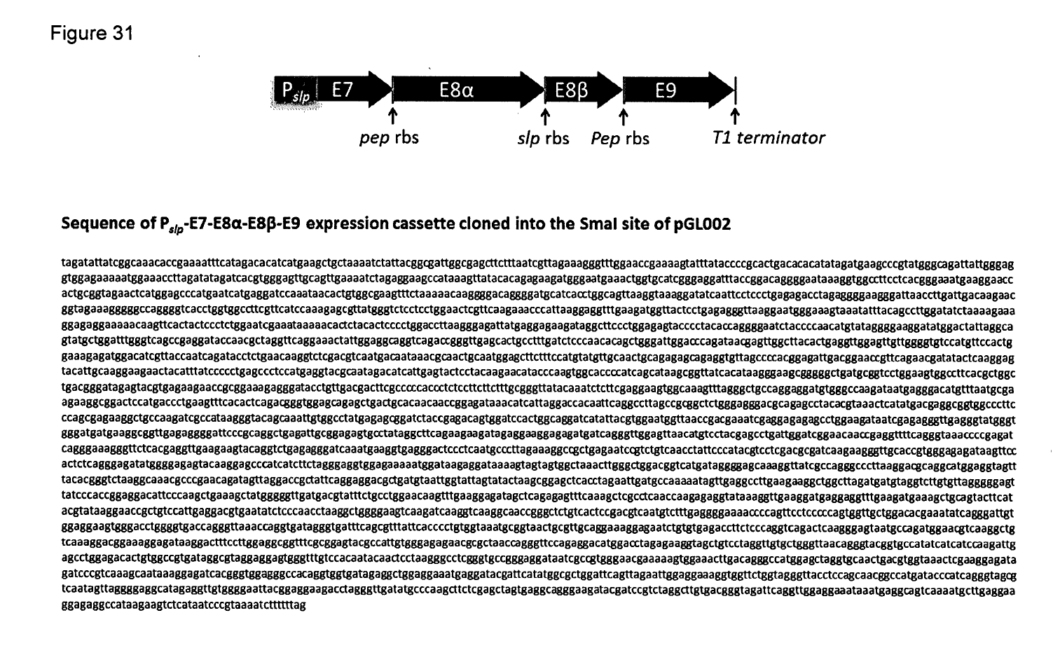

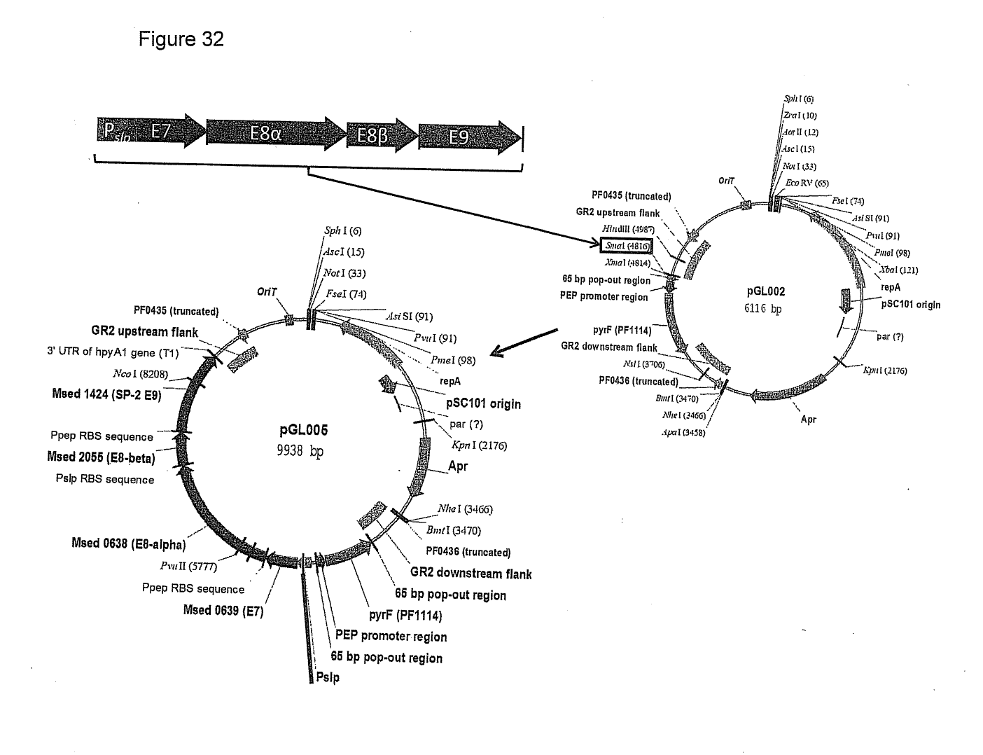

Construction of synthetic operons (SP1 and SP2B) for expression of Msed genes in P. furiosus. SOE-PCR was used to construct artificial operons for the co-expression of SP2B genes consisting of the four M. sedula genes E7 (Msed_{—}0639), E8α (Msed_{—}0638), E8β (Msed_{—}2055), E9 (Msed1424), with expression driven by the slp promoter region (Pₛₗₚ, consisting of 184 bases immediately upstream from the slp gene, PF1399). P. furiosus ribosomal binding sites from either the pep gene (5′-ggaggtttgaag (SEQ ID NO:42)) or the slp gene (FF1399, 5′-ggaggtggagaaaa(SEQ ID NO:86)) were inserted in front of each gene downstream from the first in the operon. A terminator sequence of the hpyA1 gene was included at the end of the operon (5′-aatctttttag (SEQ ID NO:54), from the 3′ UTR of PF1722) (FIG. 31). The SP2B operon construct was cloned into the SmaI site of pGL002 to make pGL005 for targeted insertion at P. furiosus genome region 2 (FIG. 32).

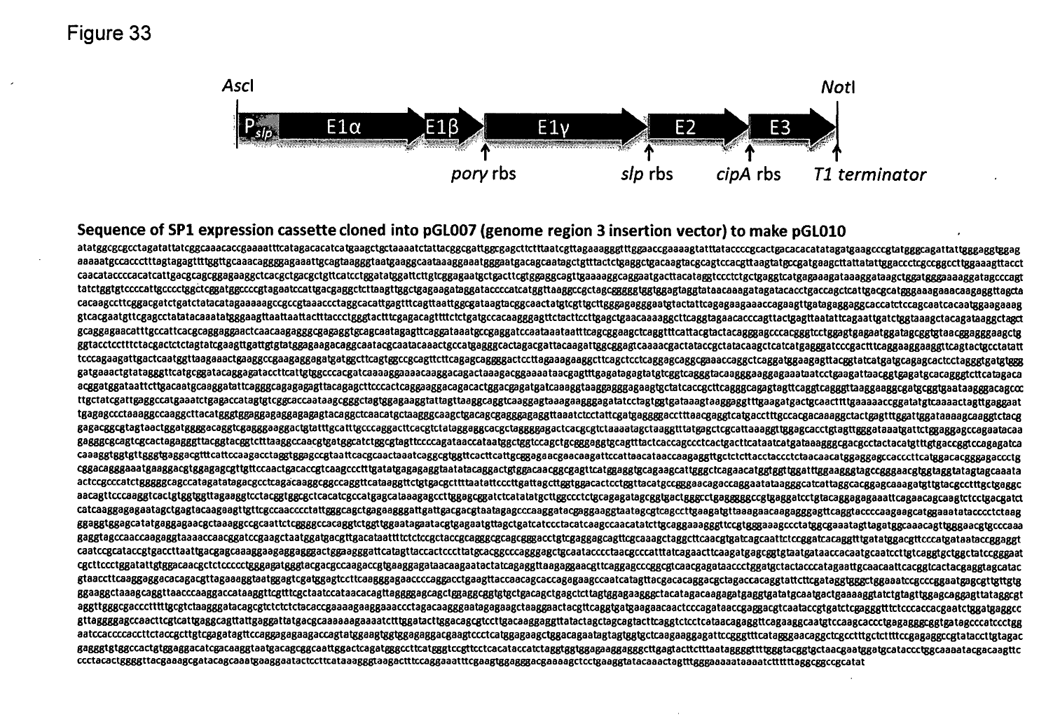

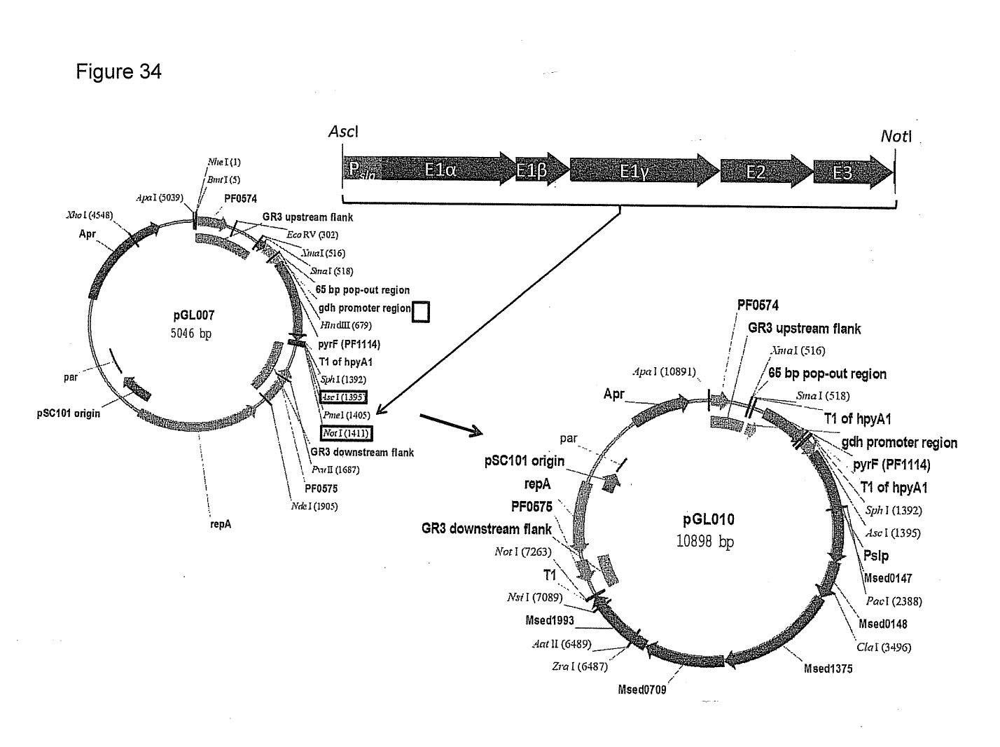

The expression cassette for SP1 consisting of the five M. sedula genes E1α (Msed_{—}0147), E1β (Msed_{—}0148), E1γ (Msed_{—}0149), E2 (Msed_{—}0709), E3 (Msed_{—}1993) was PCR-amplified from pALM506 (FIG. 33). This expression cassette contained ribosomal binding sites from the PORγ gene (PF0791, 5′-ggaggtttgaag (SEQ ID NO:42)), the slp gene (PF1399, 5′-ggaggtggagaaaa (SEQ ID NO:86)), and the cipA gene (PFO 190, 5′-ggtgatatgca (SEQ ID NO:87)). A terminator sequence was added to the 3′ end of the operon (5′-aatcttttttag (SEQ ID NO:54), from the 3′ UTR of PF 1722), and the construct was cloned into the AscI-NotI sites of pGL007 to make pGL010 (FIG. 34), for targeted insertion at P. furiosus genome region 3 (see FIG. 26).

Transformation of P. furiosus COM1 strain to yield P. furiosus strain MW56 containing SP1 and strain MW43 containing SP2B. Transformation of COM1 was performed as previously described (Lipscomb, et al., Appl Environ Microb 77:2232-2238 (2011)), except that linear plasmid DNA was used for transformation. Briefly, pGL010 and pGL005 were linearized by restriction digest and mixed (at a final concentration of ˜2 μg/mL DNA) with an aliquot of a freshly grown culture of COM1, cultured in defined cellobiose medium plus 20 μM uracil. Transformation mixtures were spread on defined cellobiose plate medium without uracil and incubated at 95° C. for ˜60 h. Transformant colonies were further purified on defined cellobiose plate medium without uracil twice. Strains were verified by PCR screening of isolated genomic DNA and sequencing of PCR products amplified from the target regions.

### Example 5

### Temperature-Dependent Production of M. sedula Enzymes in P. furiosus Using Strains PF506 (E1-E3) and MW43 (E9)

Growth of P. furiosus for biochemical assays and product analysis. P. furiosus strains were cultured in media containing 28 g/L NaCl, 3.5 g/L MgSO₄.7H₂O, 2.7 g/L MgCl₂.6 H₂O, 0.33 g/L KCl, 0.25 g/L NH₄Cl, 0.14 g/L CaCl₂.2H₂0, 2.00 mg/L FeCl₃, 0.05 mg/L H₃BO₃, 0.05 mg/L ZnCl₂, 0.03 mg/L CuCl₂.2H₂O, 0.05 mg/L MnCl₂.4H₂O, 0.05 mg/L (NH₄)₂MoO₄, 0.05 mg/L AlKSO₄.2H₂0, 0.05 mg/L CoCl₂.6 H₂0, 0.05 mg/L NiCl₂.6 H₂0, 3.30 mg/L Na₂WO₄.2H₂0, 5 g/L maltose and yeast extract, 0.5 μg/L riboflavin, and 20 μM uracil or 4 mM agmatine as needed. After these ingredients are dissolved, the media was made anaerobic by the addition of 0.5 g/L cysteine HCl, 0.5 g Na₂S (dissolved in 50 mL water). Following the reductant 1.0 g/L NaHCO₃ was added along with 1 mM potassium phosphate buffer (from a 1 M or 1000× stock at pH 6.8). If needed, the pH of the media was adjusted to 6.8 with HCl before degasing. Cultures were inoculated to 1×10⁷ cells/mL and incubated at 98° C. until cell densities reached 1×10⁸ cells/mL. Cultures were then cooled at room temperature until the temperature reached 70 to 75° C. when they were placed in an incubator set to a temperature in the range of 65 to 75° C. for up to 32 hours. Cell densities were calculated from counting a sample in a Hausser counting chamber.

P. furiosus cell paste was anaerobically resuspended in 50 mM Tris pH 8.0+ DNase 1(3 mL buffer/g cell paste). The slurry was stirred for 30 minutes in an anaerobic chamber, lysing the cells by osmotic shock. The crude extract was then centrifuges at 100,000×g for 1 hour. The resulting supernatant (S-100) was diluted (with 50 mM Tris pH 8.0) and re-concentrated 3 times with a 3 kDa centrifugation filter. The washed and concentrated S-100 was sealed in a vial to maintain anaerobicity and stored at −80° C.

Growth of M. sedula for biochemical assays and product analysis. M. sedula (DSM 5348) was grown autotrophically as described in Example 3.

M. sedula cell paste was anaerobically resuspended in 50 mM Tris pH 8.0 and Dnase 1 (2 mL buffer/g cell paste). The slurry was stirred for 1 hour in an anaerobic chamber, lysing the cells by osmotic pressure. The crude extract was then centrifuges at 100,000×g for 1 hour. The resulting supernatant (S-100) was sealed in a vial to maintain anaerobic conditions and stored at −80° C.

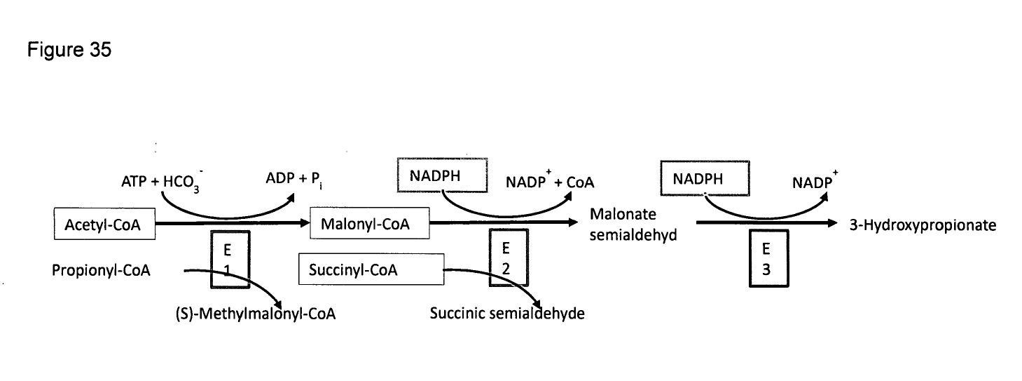

NADPH-dependent assays for the E2, E2+E3 and E1+E2+E3 reactions of SP1 (FIG. 35). All reactions were carried out in sealed anaerobic cuvettes at 75° C. containing 100 mM MOPS pH 7.5 (measured at room temperature), 5 mM MgCl₂, 5 mM DTT and the cell-free extract of P. furiosus (0.25 mg/ml). After addition of NADPH, the relevant CoA derivative and other substrates (see below), NADPH oxidation was determined by the absorbance at 340 nm and rates were calculated based on the difference before and after the addition of the CoA substrate.

E2 assay. The added substrates were 1 mM NADPH and 1 mM Succinyl-CoA. Note that E3 does not utilize succinic semialdehyde, the product of the reaction.

E2+E3 assay. The added substrates were 1 mM NADPH and 1 mM Malonyl-CoA. In this case E3 does utilize the product, malonate semialdehyde, in a NADPH-dependent reaction.

E1+E2+E3 assay. The added substrates were 1 mM NADPH, 1 mM Acetyl-CoA, 1 mM ATP and 10 mM NaHCO₃. The product, malonyl CoA, is then used by E2 and the product of that reaction, malonate semialdehyde, is then used as a substrate for E3, both in NADPH-dependent reactions.

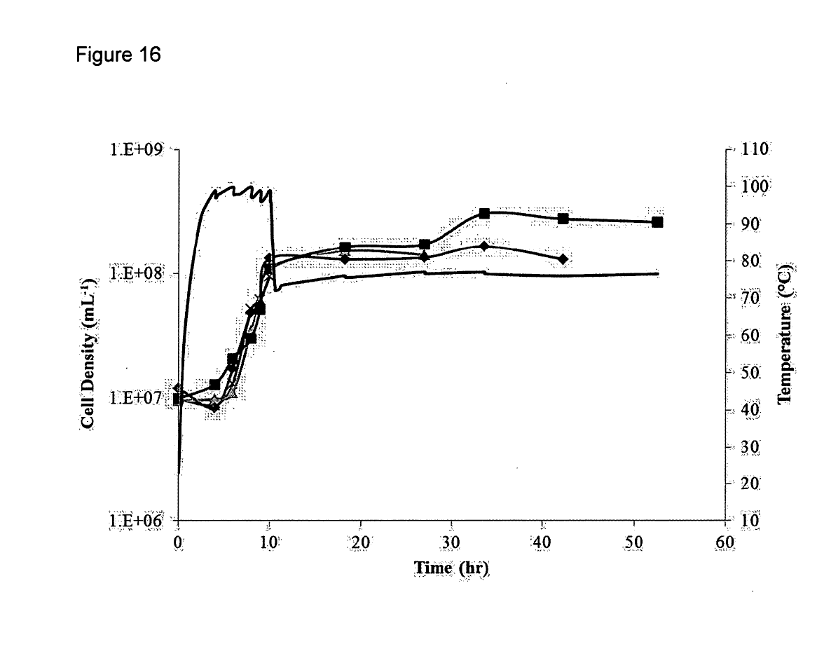

The growth of the strain PF505 before and after the temperature shift from 98° C. to 75° C. are shown in FIG. 16. The Specific activities of E1, E2 and E3 in cell-free extracts of PF506 after the temperature shift from 98° C. to 75° C. are shown in Table 9.

TABLE 9
Specific activities of E1, E2 and E3 in cell-free extracts of PF506
after the temperature shift from 98° C. to 75° C.
Specific activity: μmol NADPH oxidized/min/mg

| Enzymes | E1 + E2 + E3 | E2 + E3 | E2 |
|---|---|---|---|
| Substrate | Acetyl-CoA | Malonyl-CoA | Succinyl-CoA |
| ΔPdaD | 0 | 0 | 0 |
| 0 hr | 0.03 | 0.05 | 0.03 |
| 16 hr | 0.03 | 0.54 | 0.16 |
| 32 hr | 0.07 | 0.28 | 0.11 |
| 48 hr | 0.07 | 0.08 | 0.01 |
| Msed | 0.02 | 0.08 | 0.08 |
| Msed | 0.07 | 0.42 | 0.20 |
| (literature)* | | | |

*Published value assayed aerobically at 65° C.: Berg, I. A. et al. 2007. Science. 318, 1782-1786)

The specific activities of E1, E2 and E3 in extracts of PF506 were comparable to those measured in extracts of M. sedula and to literature values reported by others after the P. furiosus cells were grown for approx. 16 hours at 75° C. No activity was measured in cells grown at 98° C.

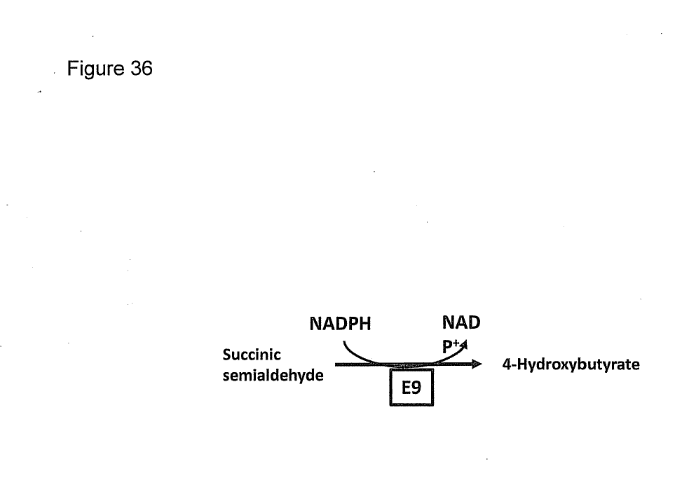

NADPH-dependent assay for E9 of the SP2B subpathway (FIG. 36). Assays were carried out in sealed anaerobic cuvettes at 75° C. containing 100 mM MOPS pH 7.5 (measured at room temperature), 5 mM MgCl₂, 5 mM DTT, 1 mM NADPH and the cell-free extract of P. furiosus (0.25 mg/ml). After addition of 1 mM succinic semialdehyde, NADPH oxidation was determined by the absorbance at 340 nm and rates were calculated based on the difference before and after the addition of the succinic semialdehyde.

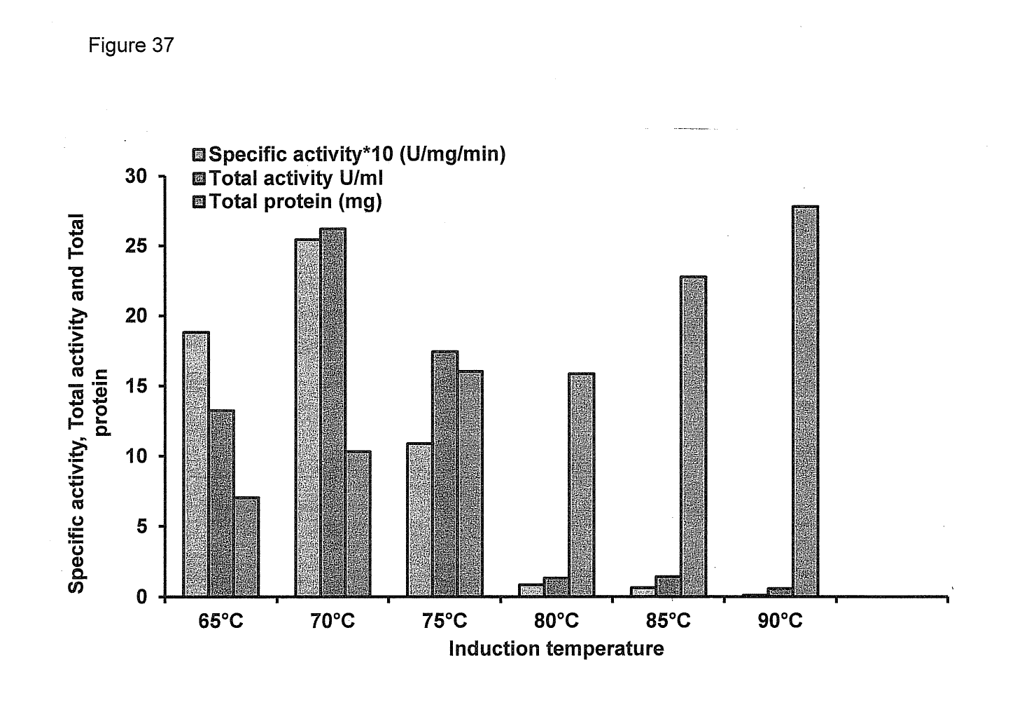

Growth of P. furiosus strain MW43 at 95° C. and temperature shift from 65° C. to 90° C. for 18 hrs (FIG. 37). Cultures were shifted from 95° C. and were incubated for 18 hr before harvesting. The maximum activity and specific activity for E9 is seen after the cultures are shifted to 70° C. (for 18 hr), with lower values at 65 and 75° C. The production of active E9 decreases dramatically at 80° C. and above.

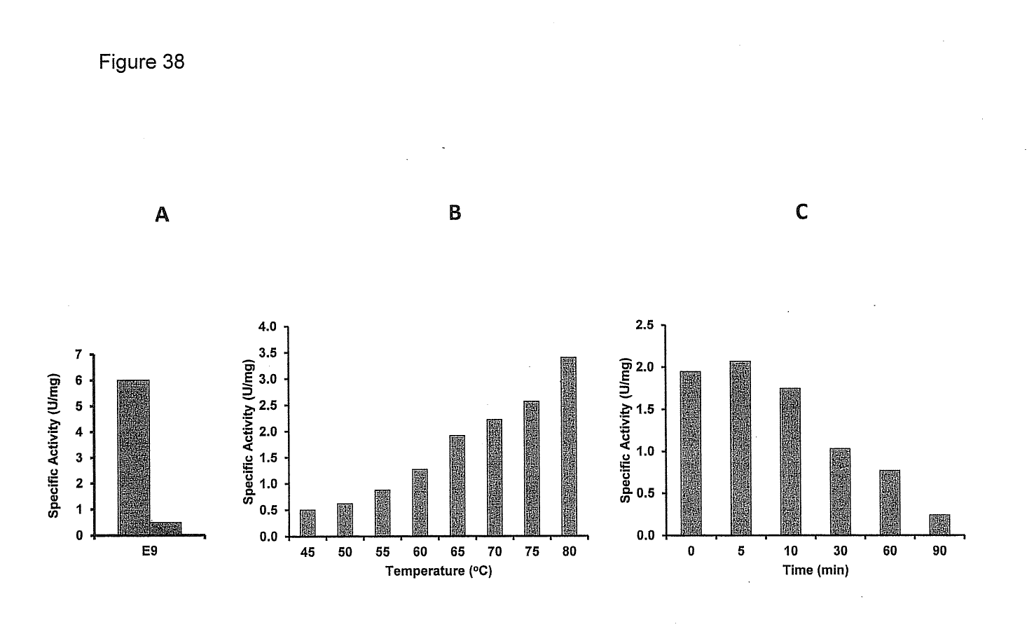

E9 temperature profile and stability in cell-free extracts of P. furiosus strain MW43 (FIG. 38). The specific activity of E9 in P. furiosus strain MW43 (grown at 70° C.) is about 10-fold higher that than measured in M. sedula. The highest E9 specific activity was measured in MW43 cells grown at 70° C. even though in cell extracts the maximum activity was above 80° C. and the enzyme has a half-life of ˜30 min at 90° C. It was concluded that P. furiosus cells should be temperature shifted from 95-98° C. to 70° C. for 18 hrs to obtain the highest activities of M. sedula enzymes.

### Example 6

### Determination of E1 and E2 Activities in P. furiosus Strain PF506, its Parent Strain ΔPdaD, in P. furiosus Strain MW56 and its Parent Strain COM1, and in M. sedula

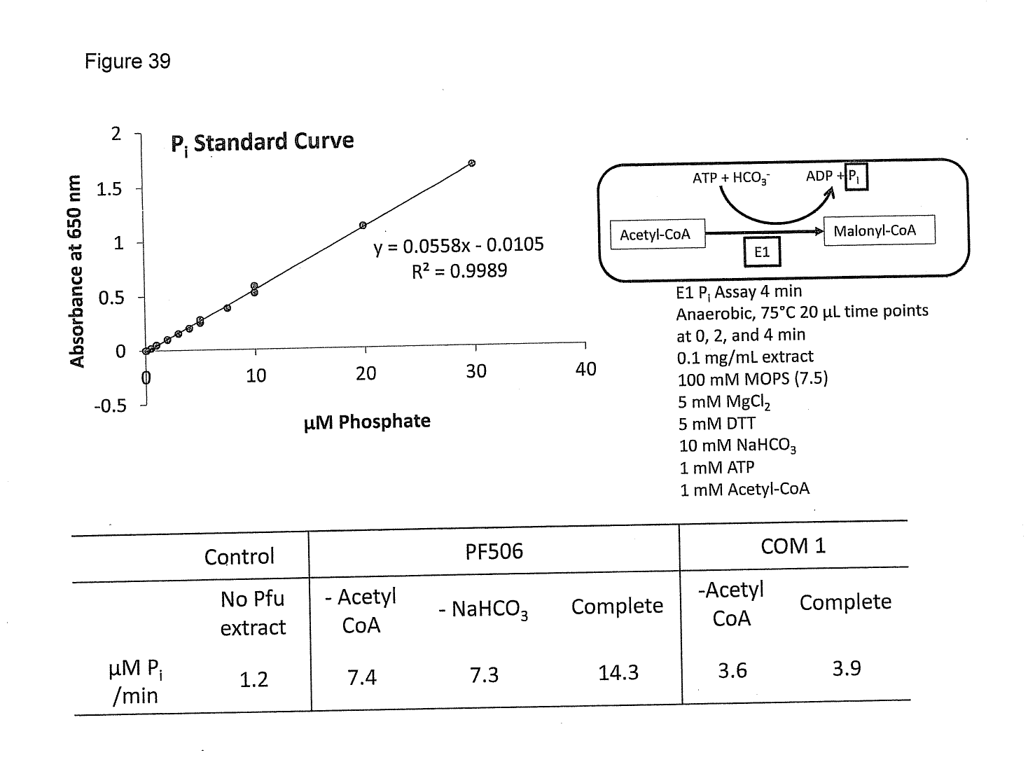

Phosphate Assay for E1 (FIG. 39). Pf extract was added to 0.1 mg/mL in buffer containing 100 mM MOPS pH 7.5 (at room temperature), 5 mM MgCl₂, and 5 mM DTT. Added substrates were 10 mM NaHCO₃, 1 mM ATP, and 1 mM Acetyl-CoA. The sealed anaerobic vials were incubated at 75° C. and 20 μL samples were taken out at 0, 2, and 4 minutes and added to a 96 well plate. The samples were diluted with 180 μL of water before the addition of 30 μl, of BioVision (Mountain View, Calif.) phosphate assay reagent. Absorbance at 650 nm was measured and rates were calculated based on the difference between the -Acetyl-CoA control for each sample.

Specific activities of E1 and E2 in cell-free extracts of recombinant and parent P. furiosus strains and in M. sedula (Table 10). The E1 and E2 assays were carried out at 75° C. as described in FIGS. 16 and 23, respectively. Specific activities are expressed as nmol phosphate released and nmol NADPH oxidized/min/mg, respectively.

TABLE 10

| | E1: | E2: |
|---|---|---|
| | Acetyl-CoA | Malonyl-CoA |
| Cell-extract | Pᵢ release | NADPH oxidation |
| COM1 | <5 | <5 |
| ΔPdaD | <5 | <5 |
| MW56 | 93 ± 10 | 92 |
| | (n = 4) | (n = 1) |
| PF506 | 74 ± 19 | 248 ± 123 |
| | (n = 6) | (n = 11) |
| Msed | 206 ± 49 | 143 ± 60 |
| | (n = 3) | (n = 3) |

The specific activities of E1 and E2 in P. furiosus strains PF506 and MW56 are comparable to those measured in Msed but are not detected in the P. furiosus parent strains.

### Example 7

### Production of 3HP by Cell-Free Extracts of P. furiosus Strains PF506 and MW56

Identification and quantitation of 3-hydroxypropionate produced by the SP 1 pathway in cell-free extracts of P. furiosus strain PF506 and strain MW56. Two approaches were used to produce 3HP: 1. Using malonyl CoA with NADPH or H₂/NADP as the electron donor catalyzed by enzymes E2+E3 (and SHI to activate H₂); and 2. Using acetyl CoA plus CO₂ (bicarbonate) with NADPH or H₂/NADP as the electron donor catalyzed by enzymes E1+E2+E3 (and SHI to activate H₂).

Detection and quantitation of 3-hydroxypropionate (3HP). 3HP produced in cell-free extracts of P. furiosus was derivatized by two reactions and each derivative was identified and quantitated by different approaches.

HPLC: 2-Nitrophenylhydrazine derivatization. The 3HP-hydrazide was prepared and extracted from mixtures with ether. The ether-extracted 3HP-hydrazide was identified by ESI-MS analysis. The ether-extracted 3HP-hydrazide was quantitated after separation by HPLC.
GC-MS: per-O-trimethylsilylate derivatization. The 3HP-TMS derivative was both identified and quantitated using GC-MS analysis.

Methods used to identify 3-HP in cell-free extracts of P. furiosus. Production of 3-HP from malonyl CoA by E2+E3 and from acetyl CoA by E1+E2+E3. To the Pf extract (0.25 mg/mL) in buffer containing 100 mM MOPS pH 7.5, 5 mM MgCl₂, and 5 mM DTT, was added 1-2 mM Malonyl-CoA (for E2+E3) or 10 mM NaHCO₃, 2 mM ATP and 1 mM Acetyl-CoA (for E1+E2+E3). The electron source was 2 mM NADPH or 0.5 mM NADP⁺ with 100% H₂ in the headspace. Sealed anaerobic vials were incubated at 75° C. for up to 2 hours.

GC-MS detection of 3-HP. A sample of the enzyme assay mixture was spiked with 20 μg of inositol as an internal standard. For hydrolysis of proteins, the samples were freeze-dried, then incubated in 2 M TFA at 80° C. for 1 hour then dried under nitrogen. The samples were then per-O-trimethylsilylated by treatment with Tri-Sil (Pierce) at 80° C. for 30 minutes. GC-MS analysis of the TMS derivatives was performed on an AT 7890n GC interfaced to a 5975C MSD, using a Grace EC-1 column (30 m×0.25 mm). The exact mass of 3-HP-TMS is 162.

2-Nitrophenyl hydrazine derivatization of 3HP. The steps to derivatize 3HP were as follows. 1) Add 100 μL it sample of cell-free extract to 200 μL ethanol. 2) Add 200 μL 20 mM 2-nitrophenyl hydrazine in 100 mM HCL/ethanol (1:1). 3) Add 200 μL 250 mM 1-Ethyl-3-(3-Dimethylaminopropyl)-N′-ethylCarbodiimide hydrochloride (1-EDC.HCL) in 3% pyridine in ethanol (v/v). 4) Heat sample at 60° C. for 20 minutes. 5) Add 100 μL of 15% (W/V) KOH. 6) Heat again at 60° C. for 15 minutes. 7) Let sample cool and acidify with 50% HCL to pH between 4-6. 8) Analyze 10-50 μL aliquots on the HPLC.

Ether extraction of 3HP-Hydrazide. This was accomplished by the following steps. 1) Add 1 mL 1 M KPO₄ Buffer, pH 7.0 to cooled 800 μL derivatized sample. 2) Add 1 mL of ether to sample and mix well. 3) Centrifuge 10 min 6,000 g to separate the phases. 4) Remove top ether layer and transfer to a new tube. 5) Repeat steps 2-4. 6) Evaporate the ether. 7) Suspend the dried sample in 200 μL methanol or 0.05% TFA. 8) Run 10-50 μL aliquots on the HPLC.

HPLC detection of 3-HP-Hydrazide. The column and run conditions were as follows: column, Supelco LiChrosorb RP-8 (5 μm); solvent system, A 0.05% TFA, B 100% acetonitrile; gradient 0-20 min, 0-100% B, 20-22 min: 100% B; flow rate: 1 ml/min; temperature: 30° C. ESI-MS detection of 3-HP-hydrazide. The derivatized 3HP samples were extracted with ether, dried, and re-constituted in methanol. The resulting samples were analyzed by direct injection on a Perkin-Elmer API 1 plus in negative mode. The exact mass of the anion 3HP-Hydrazide is 224.

Summary of methods used to identify 3-HP in cell-free extracts of P. furiosus is shown in Table _. Summary of amounts of 3-HP produced by cell-free extracts of PF506 and MW56 using malonyl CoA (E2+E3) or acetyl CoA+CO₂ (E1+E2+E3) as the carbon sources with NADPH or H₂ as the electron donor is shown in Table 11 and Table 12.

TABLE 11

| | E2 + E3: | | E1 + E2 + E3: | |
|---|---|---|---|---|
| | 1 mM Malonyl-CoA | | 1 mM Acetyl-CoA | |
| | NADPH e⁻ | H₂ e⁻ | NADPH e⁻ | H₂ e⁻ |
| | donor | donor (100% | donor | donor (100% |
| Strain | (2 mM) | headspace) | (2 mM) | headspace) |
| MW56 | HPLC | (not done) | HPLC | HPLC |
| PF506 | HPLC | CG-MS | HPLC | ESI-MS |
| | GC-MS | | ESI-MS | |
| | ESI-MS | | | |

TABLE 12

| | E2 + E3: 1 mM Malonyl-CoA | | E1 − E2 + E3: 1 mM Acetyl-CoA | |
|---|---|---|---|---|
| | NADPH e⁻ | H₂ e⁻ donor | NADPH e⁻ | H₂ e⁻ donor |
| P. furiosus | donor | (100% | donor | (100% |
| strain | (2 mM) | headspace) | (2 mM) | headspace) |
| MW0056 | 100 μM/30 min (C) | (not done) | 40 μM/8 min (D) | 48 μM/8 min (D) |
| PF506 | 160 μM/2 hr (A) | 150 μM/2 hr (A) | 50 μM/2 min (D) | 23 μM/2 min (D) |
| | 500 μM/2 hr (B) | | | |
| | 80 μM/30 min (C) | | | |

| | A | B | C | D |
|---|---|---|---|---|
| Method | GC-MS | HPLC | HPLC | HPLC |
| [Protein] | 0.25 mg/mL | 0.25 mg/mL | 0.3 mg/mL | 3 mg/mL |

### Example 8

### Production of 3HP by whole cells of P. furiosus strains PF506 and MW56

Product Analysis of E1+E2+E3 Activities in Whole Cells of P. furiosus.

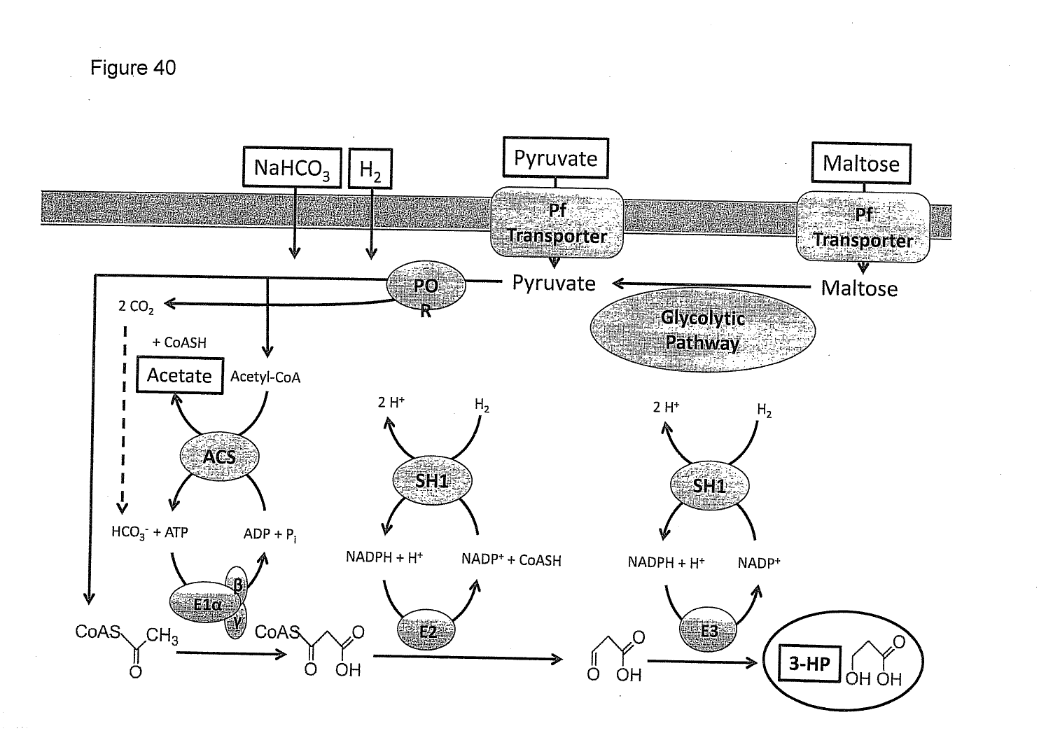

In vivo 3-HP production assay. PF506 and MW56 were grown in 2 L cultures at 98° C. for 10 hours until cell densities reached 1×10⁸ cells/mL when they were cooled and incubated at 75° C. for 16 hours. Harvested cells were suspended to 5×10¹⁰ cells/mL in 100 mM MOPS pH 7.5 and 1×Pf base salts (28 g/L NaCl, 3.5 g/L MgSO₄.7H₂O, 2.7 g/L MgCl₂.6 H₂O, 0.33 g/L KCl, 0.25 g/L NH₄Cl, 0.14 g/L CaCl₂.2H₂0). The cell suspension was sealed in a serum vial, degasses with Ar, and brought to 0.5 g/L cysteine HCl. Added substrates were 10 mM NaHCO₃ and either 10 mM maltose or 40 mM pyruvate. The vials were then degassed with H₂ and incubated at 75° C. for 60 minutes. Samples for 3-HP analysis by HPLC include a direct sample of the cell suspension, the supernatant of a portion, and the pellet re-suspended and lysed in water. A schematic of how P. furiosus metabolizes maltose and provides acetyl CoA for 3HP production is shown at FIG. 40.

A total of 135 μM of 3HP was produced by a cell suspension of MW56 (5×10¹⁰ cells/ml) after 60 min at 75° C. A total of 199 μM of 3HP was produced by a cell suspension of PF506 (5×10¹⁰ cells/ml) after 60 min at 75° C. 3-HP production by whole cells of P. furiosus strains PF506 and MW56 is summarized in Table 13. The majority (˜70%) of in vivo produced 3-HP was contained within intact cells.

TABLE 13

| | | | PF506 | | |
|---|---|---|---|---|---|
| 1 mL cell | MW56 | | | | No |
| suspension | Pyruvate | Maltose | Pyruvate | Maltose | Substrate |
| Extracellular | 45 nmol | 30 nmol | <20 nmol | 45 nmol | <20 nmol |
| Intracellular | 110 nmol | 80 nmol | 50 nmol | 100 nmol | <20 nmol |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method comprising:
providing a genetically engineered archaeon, wherein the genetically engineered archaeon comprises a heterologous polynucleotide comprising a promoter operably linked to a coding region;
culturing the genetically engineered archaeon at a first temperature within 10° C. of optimum growth temperature (Tₒₚₜ) of the genetically engineered archaeon;
shifting the culture to a second temperature that is at least 20° C. below the Tₒₚₜ of the genetically engineered archaeon; and
maintaining the genetically engineered archaeon at the second temperature, wherein activity in the genetically engineered archaeon of a polypeptide encoded by the coding region is increased compared to the activity in the genetically engineered archaeon of the polypeptide during growth at the first temperature.

2. (canceled)

3. The method of claim 1 wherein the genetically engineered archaeon is Thermococcus kodakarensis, T. onnurineus, Sulfolobus solfataricus, S. islandicus, S. acidocaldarius, or Pyrococcus furiosus.

4-6. (canceled)

7. The method of claim 1 wherein the promoter is a constitutive promoter.

8. The method of claim 1 wherein the promoter is a heterologous promoter.

9. The method of claim 1 wherein the promoter is an archaeal promoter.

10. The method of claim 1 wherein the promoter is a bacterial promoter, and wherein the genetically engineered archaeon further comprises coding regions encoding a bacterial RNA polymerase that binds to the bacterial promoter.

11. The method of claim 10 wherein the coding regions encoding the bacterial RNA polymerase are operably linked to an archaeal promoter.

12-13. (canceled)

14. The method of claim 1 wherein the maintaining comprises culturing the genetically engineered archaeon at the second temperature for at least 15 hours.

15. The method of claim 1 further comprising shifting the culture after the maintaining to the first temperature and culturing the genetically engineered archaeon at the first temperature.

16. The method of claim 15 further comprising shifting the culture to the second temperature.

17. The method of claim 1 wherein the polypeptide encoded by the coding region has an optimum activity at a temperature that is at least 20° C. below the Tₒₚₜ of the genetically engineered archaeon.

18. (canceled)

19. The method of claim 1 wherein the genetically engineered archaeon comprises more than one heterologous polynucleotide, wherein each heterologous polynucleotide comprises at least one promoter operably linked to a coding region.

20. A method comprising:
providing a cell-free extract of a genetically engineered archaeon, wherein the genetically engineered archaeon comprises a heterologous polynucleotide comprising a promoter operably linked to a coding region;
incubating the cell-free extract at a first temperature within 10° C. of optimum growth temperature (Tₒₚₜ) of the genetically engineered archaeon;
incubating the cell-free extract at a second temperature that is at least 20° C. below the Tₒₚₜ of the genetically engineered archaeon; and
maintaining the cell-free extract at the second temperature, wherein activity of a polypeptide encoded by the coding region is increased compared to the activity of the polypeptide during incubation at the first temperature.

21. A genetically engineered archaeon comprising a heterologous polynucleotide comprising a promoter operably linked to a coding region, wherein the polypeptide encoded by the coding region has an optimum activity at a temperature that is at least 20° C. below the optimum growth temperature of the genetically engineered archaeon.

22. The genetically engineered archaeon of claim 21 wherein the promoter is a constitutive promoter.

23. The genetically engineered archaeon of claim 21 wherein the promoter is a heterologous promoter.

24. The genetically engineered archaeon of claim 21 wherein the promoter is an archaeal promoter.

25. The genetically engineered archaeon of claim 21 wherein the promoter is a bacterial promoter, and wherein the genetically engineered archaeon further comprises coding regions encoding a bacterial RNA polymerase that binds to the bacterial promoter.

26. The genetically engineered archaeon of claim 25 wherein the coding regions encoding the bacterial RNA polymerase are operably linked to an archaeal promoter.

27-28. (canceled)

29. The genetically engineered archaeon of claim 21 wherein the genetically engineered archaeon comprises more than one heterologous polynucleotide, wherein each heterologous polynucleotide comprises at least one promoter operably linked to a coding region.
